SEQUENCE LISTING (104 sequences)

SEQ ID NO: 1 - protein, 196 aa, Artificial Sequence
  Recombinant Fluorescent Protein Sequence
XXXGEELFTGVVPILIELDGDVNGHKFFVRGEGEGDATXGKLSLKFICTTGKLPVPWPTLVTTLTYGVQCFSRYPDHMKRHDFFKSAMPEGYVQERTIXFKDDGTYKTRAEVKFEGDTLVNRIELKGIDFKEDGNILGHKLEYNFNXHXVYITADKQXNGIKANFTIRHNVEDGSVQLAXHYQQNTPIGDGPVXXX

SEQ ID NO: 2 - protein, 196 aa, Artificial Sequence
  Recombinant Fluorescent Protein Sequence
MRKGEELFTGVVPILIELDGDVNGHKFFVRGEGEGDATNGKLSLKFICTTGKLPVPWPTLVTTLTYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFTIRHNVEDGSVQLADHYQQNTPIGDGPVLLP

SEQ ID NO: 3 - protein, 196 aa, Artificial Sequence
  Recombinant Fluorescent Protein Sequence
MRKGEELFTGVVPILIELDGDVNGHKFFVRGEGEGDATIGKLSLKFICTTGKLPVPWPTLVTTLTYGVQCFSRYPDHMKRHDFFKSAMPEGYVQERTIYFKDDGTYKTRAEVKFEGDTLVNRIELKGIDFKEDGNILGHKLEYNFNSHKVYITADKQNNGIKANFTIRHNVEDGSVQLADHYQQNTPIGDGPVLLP

SEQ ID NO: 4 - protein, 196 aa, Artificial Sequence
  Recombinant Fluorescent Protein Sequence
MRKGEELFTGVVPILIELDGDVNGHKFFVRGEGEGDATIGKLSLKFICTTGKLPVPWPTLVTTLTYGVQCFSRYPDHMKRHDFFKSAMPEGYVQERTIYFKDDGTYKTRAEVKFEGDTLVNRIELKGIDFKEDGNILGHKLEYNFNPHNVYITADKQKNGIKANFTIRHNVEDGSVQLAEHYQQNTPIGDGPVLLP

SEQ ID NO: 5 - DNA, 588 bp, Artificial Sequence
  Recombinant Fluorescent Protein Sequence
atgaggaaaggagaagaacttttcactggagttgtcccaattcttattgaattagatggtgatgttaatgggcacaaattttttgtccgtggagagggtgaaggtgatgctacaaacggaaaactcagccttaaatttatttgcactactggaaaactacctgttccatggccaacacttgtcactactctgacctatggtgttcaatgcttttcccgttatccggatcacatgaaacggcatgactttttcaagagtgccatgcccgaaggttatgtacaggaacgcactatatctttcaaagatgacgggacctacaagacgcgtgctgaagtcaagtttgaaggtgatacccttgttaatcgtatcgagttaaagggtattgattttaaagaagatggaaacattcttggacacaaactcgagtacaactttaactcacacaatgtatacatcacggcagacaaacaaaagaatggaatcaaagctaacttcacaattcgccacaacgttgaagatggttccgttcaactagcagaccattatcaacaaaatactccaattggcgatggccctgtccttttacca

SEQ ID NO: 6 - DNA, 588 bp, Artificial Sequence
  Recombinant Fluorescent Protein Sequence
atgaggaaaggagaagaacttttcactggagtcgtcccaattcttattgaattagatggtgatgttaatgggcacaaattttttgtccgtggagagggtgaaggtgatgctacaatcggaaaactcagccttaaatttatttgcactactggaaaactacctgttccatggccaacacttgtcactactctgacctatggtgttcaatgcttttcccgttatccggatcacatgaaacggcatgactttttcaagagtgccatgcccgaaggttatgtacaggaacgcactatatatttcaaagatgacgggacctacaagacgcgtgctgaagtcaagtttgaaggtgatacccttgttaatcgtatcgagttaaagggtattgattttaaagaagatggaaacattcttggacacaaactcgagtacaactttaactcacacaaagtatacatcacggcagacaaacaaaataatggaatcaaagctaacttcacaattcgccacaacgttgaagatggttccgttcaactagcagaccattatcaacaaaatactccaattggcgatggccctgtccttttacca

SEQ ID NO: 7 - DNA, 588 bp, Artificial Sequence
  Recombinant Fluorescent Protein Sequence
atgaggaaaggagaagaacttttcactggagttgtcccaattcttattgaattagatggtgatgttaatgggcacaaattttttgtccgtggagagggtgaaggtgatgctacaatcggaaaactcagccttaaatttatttgcactactggaaaactacctgttccatggccaacacttgtcactactctgacctatggtgttcaatgcttttcccgttatccggatcacatgaaacggcatgactttttcaagagtgccatgcccgaaggttatgtacaggaacgcactatatatttcaaagatgacgggacctacaagacgcgtgctgaagtcaagtttgaaggtgatacccttgttaatcgtatcgagttaaagggtattgattttaaagaagatggaaacattcttggacacaaactcgagtacaactttaacccacacaatgtatacatcacggcagacaaacaaaagaatggaatcaaagctaacttcacaattcgccacaatgttgaagatggttccgttcaactagcagagcattatcaacaaaatactccaatcggcgatggccctgtccttttacca

SEQ ID NO: 8 - protein, 225 aa, Artificial Sequence
  Recombinant Fluorescent Protein Sequence
EEDNMAIIKEFMRFKVHMEGSVNGHEFEIEGEGEGHPYEGTQTAKLKVTKGGPLPFAWDILSPQFMYGSKAYVKHPADIPDYLKLSFPEGFTWERVMNFEDGGVVTVTQDSSLQDGEFIYKVKLLGTNFPSDGPVMQKKTMGWEASTERMYPEDGALKGEINQRLKLKDGGHYDAEVKTTYKAKKPVQLPGAYNVDIKLDITSHNEDYTIVEQYERAEGRHSTGG

SEQ ID NO: 9 - DNA, 675 bp, Artificial Sequence
  Recombinant Fluorescent Protein Sequence
gaagaagataatatggcaattatcaaggagttcatgcgcttcaaggtgcacatggagggctccgtgaacggccacgagttcgagatcgagggcgagggcgagggccacccctacgagggcacccagaccgccaagctgaaggtgaccaagggcggccccctgcccttcgcctgggacatcctgtcccctcagttcatgtacggctccaaggcctacgtgaagcaccccgccgacatccccgactacttgaagctgtccttccccgagggcttcacgtgggagcgcgtgatgaacttcgaggacggcggcgtggtgaccgtgacccaggactcctccctgcaggacggcgagttcatctacaaggtgaagctgctcggcaccaacttcccctccgacggccccgtaatgcagaagaagaccatgggctgggaggcctccactgagcggatgtaccccgaggacggcgccctgaagggcgagatcaatcagaggctgaagctgaaggacggcggccactacgacgccgaggtcaagaccacctacaaggccaagaagcccgtgcagctgcccggcgcctacaatgtggatatcaagctggacatcacctcccacaacgaggactacaccatcgtggaacagtacgagcgcgccgagggccgccactccaccggcggt

SEQ ID NO: 10 - DNA, 675 bp, Artificial Sequence
  Recombinant Fluorescent Protein Sequence
gaggaggataacatggcaattatcaaggaatttatgcgatttaaggttcacatggagggttctgttaatgggcacgaatttgagatcgaaggagagggtgaaggtcatccttacgagggaacacagaccgctaaattgaaagtcactaaaggaggacctcttccattcgcctgggatatactttcccctcagtttatgtatggttctaaagcctatgtcaaacatccggctgacatcccagactatttgaagttgtccttccccgaaggttttacatgggaacgcgttatgaacttcgaagatggcggggtcgtcacggtgacacaggactccagcttgcaagatggtgagtttatttataaagtcaagttattaggtactaattttccatcagatggacccgttatgcagaaaaagacgatgggctgggaggcatccactgaacgcatgtacccagaagacggtgcactcaaaggtgagatcaatcaacgcctcaagcttaaagatggtggccattacgatgcagaggttaagacaacatataaggcaaaaaagcctgtccagttaccaggcgcctataacgtggacataaaattggacattacgagccataacgaggactacacaatcgtggagcagtatgagcgtgcagagggtcgtcacagtacaggtggc

SEQ ID NO: 11 - protein, 20 aa, Artificial Sequence
  Recombinant Fluorescent Protein Sequence
MGLPDNHYLSTQSVLSKDPN

SEQ ID NO: 12 - protein, 20 aa, Artificial Sequence
  Recombinant Fluorescent Protein Sequence
MDLPDNHYLSTQTILLKDLN

SEQ ID NO: 13 - protein, 20 aa, Artificial Sequence
  Recombinant Fluorescent Protein Sequence
MDLPDDHYLSTQTILSKDLN

SEQ ID NO: 14 - protein, 21 aa, Artificial Sequence
  Recombinant Fluorescent Protein Sequence
EKRDHMVLLEFVTAAGITGAS

SEQ ID NO: 15 - protein, 21 aa, Artificial Sequence
  Recombinant Fluorescent Protein Sequence
EKRDHMVLLEYVTAAGITDAS

SEQ ID NO: 16 - DNA, 39 bp, artificial sequence
  oligonucleotide primer
agatatacatatgcaacgagcgcgttgttatctgatagg

SEQ ID NO: 17 - DNA, 37 bp, artificial sequence
  oligonucleotide primer
aattcggatccattttcattgtgcagccgccacgcta

SEQ ID NO: 18 - DNA, 39 bp, artificial sequence
  oligonucleotide primer
agatatacatatgcgttttgccatcgtggtgaccgggcc

SEQ ID NO: 19 - DNA, 39 bp, artificial sequence
  oligonucleotide primer
agatatacatatgaaacgaattgcgtttgttttttctac

SEQ ID NO: 20 - DNA, 37 bp, artificial sequence
  oligonucleotide primer
aattcggatccccaggccatctggctggagtgcttaa

SEQ ID NO: 21 - protein, 8 aa, Artificial Sequence
  Peptide linker
DVGSGGGS

SEQ ID NO: 22 - protein, 8 aa, Artificial Sequence
  Peptide linker
GGGSGSGG

SEQ ID NO: 23 - protein, 8 aa, Artificial Sequence
  Peptide linker
GGGSGGGS

SEQ ID NO: 24 - protein, 5 aa, Artificial Sequence
  Peptide linker
MVSKG

SEQ ID NO: 25 - protein, 6 aa, Artificial Sequence
  Peptide linker
MDELYK

SEQ ID NO: 26 - protein, 40 aa, Artificial Sequence
  Recombinant Fluorescent Protein Sequence
DLPDDHYLSTQTILSKDLNEKRDHMVLLEYVTAAGITDAS

SEQ ID NO: 27 - DNA, 38 bp, Artificial Sequence
  oligonucleotide primer
gcaaggaatggtgcatgcaaggagatggcgcccaacag

SEQ ID NO: 28 - DNA, 39 bp, Artificial Sequence
  oligonucleotide primer
gaccatgtggtcacgcttttcgttgagatctttcgaaag

SEQ ID NO: 29 - DNA, 40 bp, Artificial Sequence
  oligonucleotide primer
cttttcgttgagatctttcgaaaggatagtttgtgtcgac

SEQ ID NO: 30 - DNA, 38 bp, Artificial Sequence
  oligonucleotide primer
ttcgaaaggatagtttgtgtcgacaggtaatggtcgtc

SEQ ID NO: 31 - DNA, 39 bp, Artificial Sequence
  oligonucleotide primer
tgtgtcgacaggtaatggtcgtctggtaaatctcctcct

SEQ ID NO: 32 - DNA, 41 bp, Artificial Sequence
  oligonucleotide primer
gtcgtctggtaaatctcctcctttagtgactttcaatttag

SEQ ID NO: 33 - DNA, 38 bp, Artificial Sequence
  oligonucleotide primer
tcgaaagatctcaacgaaaagcgtgaccacatggtcct

SEQ ID NO: 34 - DNA, 39 bp, Artificial Sequence
  oligonucleotide primer
cgaaaagcgtgaccacatggtccttcttgagtatgtaac

SEQ ID NO: 35 - DNA, 39 bp, Artificial Sequence
  oligonucleotide primer
tggtccttcttgagtatgtaactgctgctgggattacag

SEQ ID NO: 36 - DNA, 39 bp, Artificial Sequence
  oligonucleotide primer
gtaactgctgctgggattacagatgcatctcctcttcca

SEQ ID NO: 37 - DNA, 40 bp, Artificial Sequence
  oligonucleotide primer
attacagatgcatctcctcttccattcgcctgggatatac

SEQ ID NO: 38 - DNA, 30 bp, Artificial Sequence
  oligonucleotide primer
gaggcctctagaggttatgctagttattgc

SEQ ID NO: 39 - DNA, 38 bp, Artificial Sequence; identical to SEQ ID NO: 27
  oligonucleotide primer

SEQ ID NO: 40 - DNA, 39 bp, Artificial Sequence; identical to SEQ ID NO: 28
  oligonucleotide primer

SEQ ID NO: 41 - DNA, 40 bp, Artificial Sequence; identical to SEQ ID NO: 29
  oligonucleotide primer

SEQ ID NO: 42 - DNA, 38 bp, Artificial Sequence; identical to SEQ ID NO: 30
  oligonucleotide primer

SEQ ID NO: 43 - DNA, 41 bp, Artificial Sequence
  oligonucleotide primer
tgtgtcgacaggtaatggtcgtctggtaaatcatctttaag

SEQ ID NO: 44 - DNA, 40 bp, Artificial Sequence
  oligonucleotide primer
gtcgtctggtaaatcatctttaagcttgaggcgttgattg

SEQ ID NO: 45 - DNA, 38 bp, Artificial sequence; identical to SEQ ID NO: 33
  oligonucleotide primer

SEQ ID NO: 46 - DNA, 39 bp, Artificial Sequence; identical to SEQ ID NO: 34
  oligonucleotide primer

SEQ ID NO: 47 - DNA, 39 bp, Artificial Sequence; identical to SEQ ID NO: 35
  oligonucleotide primer

SEQ ID NO: 48 - DNA, 39 bp, Artificial Sequence
  oligonucleotide primer
gtaactgctgctgggattacagatgcatctggtggccat

SEQ ID NO: 49 - DNA, 40 bp, Artificial Sequence
  oligonucleotide primer
attacagatgcatctggtggccattacgatgcagaggtta

SEQ ID NO: 50 - DNA, 30 bp, Artificial Sequence; identical to SEQ ID NO: 38
  oligonucleotide primer

SEQ ID NO: 51 - DNA, 723 bp, Artificial sequence
  Recombinant Fluorescent Protein Sequence
atggggcacaaattttttgtccgtggagagggtgaaggtgatgctacaatcggaaaactcagccttaaatttatttgcactactggaaaactacctgttccatggccaacacttgtcactactctgacctatggtgttcaatgcttttcccgttatccggatcacatgaaacggcatgactttttcaagagtgccatgcccgaaggttatgtacaggaacgcactatatatttcaaagatgacgggacctacaagacgcgtgctgaagtcaagtttgaaggtgatacccttgttaatcgtatcgagttaaagggtattgattttaaagaagatggaaacattcttggacacaaactcgagtacaactttaactcacacaaagtatacatcacggcagacaaacaaaataatggaatcaaagctaacttcacaattcgccacaacgttgaagatggttccgttcaactagcagaccattatcaacaaaatactccaattggcgatggccctgtccttttaccagacgaccattacctgtcgacacaaactatcctttcgaaagatctcaacgaaaagcgtgaccacatggtccttcttgagtatgtaactgctgctgggattacagatgctagcggtggagggtctggtggcggatcaaggaaaggagaagaacttttcactggagtcgtcccaattcttattgaattagatggtgatgttaat

SEQ ID NO: 52 - DNA, 723 bp, Artificial sequence
  Recombinant Fluorescent Protein Sequence
atgggaaaactcagccttaaatttatttgcactactggaaaactacctgttccatggccaacacttgtcactactctgacctatggtgttcaatgcttttcccgttatccggatcacatgaaacggcatgactttttcaagagtgccatgcccgaaggttatgtacaggaacgcactatatatttcaaagatgacgggacctacaagacgcgtgctgaagtcaagtttgaaggtgatacccttgttaatcgtatcgagttaaagggtattgattttaaagaagatggaaacattcttggacacaaactcgagtacaactttaactcacacaaagtatacatcacggcagacaaacaaaataatggaatcaaagctaacttcacaattcgccacaacgttgaagatggttccgttcaactagcagaccattatcaacaaaatactccaattggcgatggccctgtccttttaccagacgaccattacctgtcgacacaaactatcctttcgaaagatctcaacgaaaagcgtgaccacatggtccttcttgagtatgtaactgctgctgggattacagatgctagcggtggagggtctggtggcggatcaaggaaaggagaagaacttttcactggagtcgtcccaattcttattgaattagatggtgatgttaatgggcacaaattttttgtccgtggagagggtgaaggtgatgctacaatc

SEQ ID NO: 53 - DNA, 723 bp, Artificial sequence
  Recombinant Fluorescent Protein Sequence
atgaaactacctgttccatggccaacacttgtcactactctgacctatggtgttcaatgcttttcccgttatccggatcacatgaaacggcatgactttttcaagagtgccatgcccgaaggttatgtacaggaacgcactatatatttcaaagatgacgggacctacaagacgcgtgctgaagtcaagtttgaaggtgatacccttgttaatcgtatcgagttaaagggtattgattttaaagaagatggaaacattcttggacacaaactcgagtacaactttaactcacacaaagtatacatcacggcagacaaacaaaataatggaatcaaagctaacttcacaattcgccacaacgttgaagatggttccgttcaactagcagaccattatcaacaaaatactccaattggcgatggccctgtccttttaccagacgaccattacctgtcgacacaaactatcctttcgaaagatctcaacgaaaagcgtgaccacatggtccttcttgagtatgtaactgctgctgggattacagatgctagcggtggagggtctggtggcggatcaaggaaaggagaagaacttttcactggagtcgtcccaattcttattgaattagatggtgatgttaatgggcacaaattttttgtccgtggagagggtgaaggtgatgctacaatcggaaaactcagccttaaatttatttgcactactgga

SEQ ID NO: 54 - DNA, 723 bp, Artificial sequence
  Recombinant Fluorescent Protein Sequence
atgtatgtacaggaacgcactatatatttcaaagatgacgggacctacaagacgcgtgctgaagtcaagtttgaaggtgatacccttgttaatcgtatcgagttaaagggtattgattttaaagaagatggaaacattcttggacacaaactcgagtacaactttaactcacacaaagtatacatcacggcagacaaacaaaataatggaatcaaagctaacttcacaattcgccacaacgttgaagatggttccgttcaactagcagaccattatcaacaaaatactccaattggcgatggccctgtccttttaccagacgaccattacctgtcgacacaaactatcctttcgaaagatctcaacgaaaagcgtgaccacatggtccttcttgagtatgtaactgctgctgggattacagatgctagcggtggagggtctggtggcggatcaaggaaaggagaagaacttttcactggagtcgtcccaattcttattgaattagatggtgatgttaatgggcacaaattttttgtccgtggagagggtgaaggtgatgctacaatcggaaaactcagccttaaatttatttgcactactggaaaactacctgttccatggccaacacttgtcactactctgacctatggtgttcaatgcttttcccgttatccggatcacatgaaacggcatgactttttcaagagtgccatgcccgaaggt

SEQ ID NO: 55 - DNA, 723 bp, Artificial sequence
  Recombinant Fluorescent Protein Sequence
atggacgggacctacaagacgcgtgctgaagtcaagtttgaaggtgatacccttgttaatcgtatcgagttaaagggtattgattttaaagaagatggaaacattcttggacacaaactcgagtacaactttaactcacacaaagtatacatcacggcagacaaacaaaataatggaatcaaagctaacttcacaattcgccacaacgttgaagatggttccgttcaactagcagaccattatcaacaaaatactccaattggcgatggccctgtccttttaccagacgaccattacctgtcgacacaaactatcctttcgaaagatctcaacgaaaagcgtgaccacatggtccttcttgagtatgtaactgctgctgggattacagatgctagcggtggagggtctggtggcggatcaaggaaaggagaagaacttttcactggagtcgtcccaattcttattgaattagatggtgatgttaatgggcacaaattttttgtccgtggagagggtgaaggtgatgctacaatcggaaaactcagccttaaatttatttgcactactggaaaactacctgttccatggccaacacttgtcactactctgacctatggtgttcaatgcttttcccgttatccggatcacatgaaacggcatgactttttcaagagtgccatgcccgaaggttatgtacaggaacgcactatatatttcaaagat

SEQ ID NO: 56 - DNA, 723 bp, Artificial sequence
  Recombinant Fluorescent Protein Sequence
atgacccttgttaatcgtatcgagttaaagggtattgattttaaagaagatggaaacattcttggacacaaactcgagtacaactttaactcacacaaagtatacatcacggcagacaaacaaaataatggaatcaaagctaacttcacaattcgccacaacgttgaagatggttccgttcaactagcagaccattatcaacaaaatactccaattggcgatggccctgtccttttaccagacgaccattacctgtcgacacaaactatcctttcgaaagatctcaacgaaaagcgtgaccacatggtccttcttgagtatgtaactgctgctgggattacagatgctagcggtggagggtctggtggcggatcaaggaaaggagaagaacttttcactggagtcgtcccaattcttattgaattagatggtgatgttaatgggcacaaattttttgtccgtggagagggtgaaggtgatgctacaatcggaaaactcagccttaaatttatttgcactactggaaaactacctgttccatggccaacacttgtcactactctgacctatggtgttcaatgcttttcccgttatccggatcacatgaaacggcatgactttttcaagagtgccatgcccgaaggttatgtacaggaacgcactatatatttcaaagatgacgggacctacaagacgcgtgctgaagtcaagtttgaaggtgat

SEQ ID NO: 57 - DNA, 723 bp, Artificial sequence
  Recombinant Fluorescent Protein Sequence
atgtttaaagaagatggaaacattcttggacacaaactcgagtacaactttaactcacacaaagtatacatcacggcagacaaacaaaataatggaatcaaagctaacttcacaattcgccacaacgttgaagatggttccgttcaactagcagaccattatcaacaaaatactccaattggcgatggccctgtccttttaccagacgaccattacctgtcgacacaaactatcctttcgaaagatctcaacgaaaagcgtgaccacatggtccttcttgagtatgtaactgctgctgggattacagatgctagcggtggagggtctggtggcggatcaaggaaaggagaagaacttttcactggagtcgtcccaattcttattgaattagatggtgatgttaatgggcacaaattttttgtccgtggagagggtgaaggtgatgctacaatcggaaaactcagccttaaatttatttgcactactggaaaactacctgttccatggccaacacttgtcactactctgacctatggtgttcaatgcttttcccgttatccggatcacatgaaacggcatgactttttcaagagtgccatgcccgaaggttatgtacaggaacgcactatatatttcaaagatgacgggacctacaagacgcgtgctgaagtcaagtttgaaggtgatacccttgttaatcgtatcgagttaaagggtattgat

SEQ ID NO: 58 - DNA, 723 bp, Artificial sequence
  Recombinant Fluorescent Protein Sequence
atgctcgagtacaactttaactcacacaaagtatacatcacggcagacaaacaaaataatggaatcaaagctaacttcacaattcgccacaacgttgaagatggttccgttcaactagcagaccattatcaacaaaatactccaattggcgatggccctgtccttttaccagacgaccattacctgtcgacacaaactatcctttcgaaagatctcaacgaaaagcgtgaccacatggtccttcttgagtatgtaactgctgctgggattacagatgctagcggtggagggtctggtggcggatcaaggaaaggagaagaacttttcactggagtcgtcccaattcttattgaattagatggtgatgttaatgggcacaaattttttgtccgtggagagggtgaaggtgatgctacaatcggaaaactcagccttaaatttatttgcactactggaaaactacctgttccatggccaacacttgtcactactctgacctatggtgttcaatgcttttcccgttatccggatcacatgaaacggcatgactttttcaagagtgccatgcccgaaggttatgtacaggaacgcactatatatttcaaagatgacgggacctacaagacgcgtgctgaagtcaagtttgaaggtgatacccttgttaatcgtatcgagttaaagggtattgattttaaagaagatggaaacattcttggacacaaa

SEQ ID NO: 59 - DNA, 723 bp, Artificial sequence
  Recombinant Fluorescent Protein Sequence
atgaactcacacaaagtatacatcacggcagacaaacaaaataatggaatcaaagctaacttcacaattcgccacaacgttgaagatggttccgttcaactagcagaccattatcaacaaaatactccaattggcgatggccctgtccttttaccagacgaccattacctgtcgacacaaactatcctttcgaaagatctcaacgaaaagcgtgaccacatggtccttcttgagtatgtaactgctgctgggattacagatgctagcggtggagggtctggtggcggatcaaggaaaggagaagaacttttcactggagtcgtcccaattcttattgaattagatggtgatgttaatgggcacaaattttttgtccgtggagagggtgaaggtgatgctacaatcggaaaactcagccttaaatttatttgcactactggaaaactacctgttccatggccaacacttgtcactactctgacctatggtgttcaatgcttttcccgttatccggatcacatgaaacggcatgactttttcaagagtgccatgcccgaaggttatgtacaggaacgcactatatatttcaaagatgacgggacctacaagacgcgtgctgaagtcaagtttgaaggtgatacccttgttaatcgtatcgagttaaagggtattgattttaaagaagatggaaacattcttggacacaaactcgagtacaacttt

SEQ ID NO: 60 - DNA, 723 bp, Artificial sequence
  Recombinant Fluorescent Protein Sequence
atgaataatggaatcaaagctaacttcacaattcgccacaacgttgaagatggttccgttcaactagcagaccattatcaacaaaatactccaattggcgatggccctgtccttttaccagacgaccattacctgtcgacacaaactatcctttcgaaagatctcaacgaaaagcgtgaccacatggtccttcttgagtatgtaactgctgctgggattacagatgctagcggtggagggtctggtggcggatcaaggaaaggagaagaacttttcactggagtcgtcccaattcttattgaattagatggtgatgttaatgggcacaaattttttgtccgtggagagggtgaaggtgatgctacaatcggaaaactcagccttaaatttatttgcactactggaaaactacctgttccatggccaacacttgtcactactctgacctatggtgttcaatgcttttcccgttatccggatcacatgaaacggcatgactttttcaagagtgccatgcccgaaggttatgtacaggaacgcactatatatttcaaagatgacgggacctacaagacgcgtgctgaagtcaagtttgaaggtgatacccttgttaatcgtatcgagttaaagggtattgattttaaagaagatggaaacattcttggacacaaactcgagtacaactttaactcacacaaagtatacatcacggcagacaaacaa

SEQ ID NO: 61 - DNA, 723 bp, Artificial sequence
  Recombinant Fluorescent Protein Sequence
atgggttccgttcaactagcagaccattatcaacaaaatactccaattggcgatggccctgtccttttaccagacgaccattacctgtcgacacaaactatcctttcgaaagatctcaacgaaaagcgtgaccacatggtccttcttgagtatgtaactgctgctgggattacagatgctagcggtggagggtctggtggcggatcaaggaaaggagaagaacttttcactggagtcgtcccaattcttattgaattagatggtgatgttaatgggcacaaattttttgtccgtggagagggtgaaggtgatgctacaatcggaaaactcagccttaaatttatttgcactactggaaaactacctgttccatggccaacacttgtcactactctgacctatggtgttcaatgcttttcccgttatccggatcacatgaaacggcatgactttttcaagagtgccatgcccgaaggttatgtacaggaacgcactatatatttcaaagatgacgggacctacaagacgcgtgctgaagtcaagtttgaaggtgatacccttgttaatcgtatcgagttaaagggtattgattttaaagaagatggaaacattcttggacacaaactcgagtacaactttaactcacacaaagtatacatcacggcagacaaacaaaataatggaatcaaagctaacttcacaattcgccacaacgttgaagat

SEQ ID NO: 62 - DNA, 723 bp, Artificial sequence
  Recombinant Fluorescent Protein Sequence
atggatggccctgtccttttaccagacgaccattacctgtcgacacaaactatcctttcgaaagatctcaacgaaaagcgtgaccacatggtccttcttgagtatgtaactgctgctgggattacagatgctagcggtggagggtctggtggcggatcaaggaaaggagaagaacttttcactggagtcgtcccaattcttattgaattagatggtgatgttaatgggcacaaattttttgtccgtggagagggtgaaggtgatgctacaatcggaaaactcagccttaaatttatttgcactactggaaaactacctgttccatggccaacacttgtcactactctgacctatggtgttcaatgcttttcccgttatccggatcacatgaaacggcatgactttttcaagagtgccatgcccgaaggttatgtacaggaacgcactatatatttcaaagatgacgggacctacaagacgcgtgctgaagtcaagtttgaaggtgatacccttgttaatcgtatcgagttaaagggtattgattttaaagaagatggaaacattcttggacacaaactcgagtacaactttaactcacacaaagtatacatcacggcagacaaacaaaataatggaatcaaagctaacttcacaattcgccacaacgttgaagatggttccgttcaactagcagaccattatcaacaaaatactccaattggc

SEQ ID NO: 63 - DNA, 723 bp, Artificial sequence
  Recombinant Fluorescent Protein Sequence
atgccagacgaccattacctgtcgacacaaactatcctttcgaaagatctcaacgaaaagcgtgaccacatggtccttcttgagtatgtaactgctgctgggattacagatgctagcggtggagggtctggtggcggatcaaggaaaggagaagaacttttcactggagtcgtcccaattcttattgaattagatggtgatgttaatgggcacaaattttttgtccgtggagagggtgaaggtgatgctacaatcggaaaactcagccttaaatttatttgcactactggaaaactacctgttccatggccaacacttgtcactactctgacctatggtgttcaatgcttttcccgttatccggatcacatgaaacggcatgactttttcaagagtgccatgcccgaaggttatgtacaggaacgcactatatatttcaaagatgacgggacctacaagacgcgtgctgaagtcaagtttgaaggtgatacccttgttaatcgtatcgagttaaagggtattgattttaaagaagatggaaacattcttggacacaaactcgagtacaactttaactcacacaaagtatacatcacggcagacaaacaaaataatggaatcaaagctaacttcacaattcgccacaacgttgaagatggttccgttcaactagcagaccattatcaacaaaatactccaattggcgatggccctgtcctttta

SEQ ID NO: 64 - DNA, 723 bp, Artificial sequence
  Recombinant Fluorescent Protein Sequence
atgcgtgaccacatggtccttcttgagtatgtaactgctgctgggattacagatgctagcggtggagggtctggtggcggatcaaggaaaggagaagaacttttcactggagtcgtcccaattcttattgaattagatggtgatgttaatgggcacaaattttttgtccgtggagagggtgaaggtgatgctacaatcggaaaactcagccttaaatttatttgcactactggaaaactacctgttccatggccaacacttgtcactactctgacctatggtgttcaatgcttttcccgttatccggatcacatgaaacggcatgactttttcaagagtgccatgcccgaaggttatgtacaggaacgcactatatatttcaaagatgacgggacctacaagacgcgtgctgaagtcaagtttgaaggtgatacccttgttaatcgtatcgagttaaagggtattgattttaaagaagatggaaacattcttggacacaaactcgagtacaactttaactcacacaaagtatacatcacggcagacaaacaaaataatggaatcaaagctaacttcacaattcgccacaacgttgaagatggttccgttcaactagcagaccattatcaacaaaatactccaattggcgatggccctgtccttttaccagacgaccattacctgtcgacacaaactatcctttcgaaagatctcaacgaaaag

SEQ ID NO: 65 - protein, 241 aa, Artificial sequence
  Recombinant Fluorescent Protein Sequence
MGHKFFVRGEGEGDATIGKLSLKFICTTGKLPVPWPTLVTTLTYGVQCFSRYPDHMKRHDFFKSAMPEGYVQERTIYFKDDGTYKTRAEVKFEGDTLVNRIELKGIDFKEDGNILGHKLEYNFNSHKVYITADKQNNGIKANFTIRHNVEDGSVQLADHYQQNTPIGDGPVLLPDDHYLSTQTILSKDLNEKRDHMVLLEYVTAAGITDASGGGSGGGSRKGEELFTGVVPILIELDGDVN

SEQ ID NO: 66 - protein, 241 aa, Artificial sequence
  Recombinant Fluorescent Protein Sequence
MGKLSLKFICTTGKLPVPWPTLVTTLTYGVQCFSRYPDHMKRHDFFKSAMPEGYVQERTIYFKDDGTYKTRAEVKFEGDTLVNRIELKGIDFKEDGNILGHKLEYNFNSHKVYITADKQNNGIKANFTIRHNVEDGSVQLADHYQQNTPIGDGPVLLPDDHYLSTQTILSKDLNEKRDHMVLLEYVTAAGITDASGGGSGGGSRKGEELFTGVVPILIELDGDVNGHKFFVRGEGEGDATI

SEQ ID NO: 67 - protein, 241 aa, Artificial sequence
  Recombinant Fluorescent Protein Sequence
MKLPVPWPTLVTTLTYGVQCFSRYPDHMKRHDFFKSAMPEGYVQERTIYFKDDGTYKTRAEVKFEGDTLVNRIELKGIDFKEDGNILGHKLEYNFNSHKVYITADKQNNGIKANFTIRHNVEDGSVQLADHYQQNTPIGDGPVLLPDDHYLSTQTILSKDLNEKRDHMVLLEYVTAAGITDASGGGSGGGSRKGEELFTGVVPILIELDGDVNGHKFFVRGEGEGDATIGKLSLKFICTTG

SEQ ID NO: 68 - protein, 241 aa, Artificial sequence
  Recombinant Fluorescent Protein Sequence
MYVQERTIYFKDDGTYKTRAEVKFEGDTLVNRIELKGIDFKEDGNILGHKLEYNFNSHKVYITADKQNNGIKANFTIRHNVEDGSVQLADHYQQNTPIGDGPVLLPDDHYLSTQTILSKDLNEKRDHMVLLEYVTAAGITDASGGGSGGGSRKGEELFTGVVPILIELDGDVNGHKFFVRGEGEGDATIGKLSLKFICTTGKLPVPWPTLVTTLTYGVQCFSRYPDHMKRHDFFKSAMPEG

SEQ ID NO: 69 - protein, 241 aa, Artificial sequence
  Recombinant Fluorescent Protein Sequence
MDGTYKTRAEVKFEGDTLVNRIELKGIDFKEDGNILGHKLEYNFNSHKVYITADKQNNGIKANFTIRHNVEDGSVQLADHYQQNTPIGDGPVLLPDDHYLSTQTILSKDLNEKRDHMVLLEYVTAAGITDASGGGSGGGSRKGEELFTGVVPILIELDGDVNGHKFFVRGEGEGDATIGKLSLKFICTTGKLPVPWPTLVTTLTYGVQCFSRYPDHMKRHDFFKSAMPEGYVQERTIYFKD

SEQ ID NO: 70 - protein, 241 aa, Artificial sequence
  Recombinant Fluorescent Protein Sequence
MTLVNRIELKGIDFKEDGNILGHKLEYNFNSHKVYITADKQNNGIKANFTIRHNVEDGSVQLADHYQQNTPIGDGPVLLPDDHYLSTQTILSKDLNEKRDHMVLLEYVTAAGITDASGGGSGGGSRKGEELFTGVVPILIELDGDVNGHKFFVRGEGEGDATIGKLSLKFICTTGKLPVPWPTLVTTLTYGVQCFSRYPDHMKRHDFFKSAMPEGYVQERTIYFKDDGTYKTRAEVKFEGD

SEQ ID NO: 71 - protein, 241 aa, Artificial sequence
  Recombinant Fluorescent Protein Sequence
MFKEDGNILGHKLEYNFNSHKVYITADKQNNGIKANFTIRHNVEDGSVQLADHYQQNTPIGDGPVLLPDDHYLSTQTILSKDLNEKRDHMVLLEYVTAAGITDASGGGSGGGSRKGEELFTGVVPILIELDGDVNGHKFFVRGEGEGDATIGKLSLKFICTTGKLPVPWPTLVTTLTYGVQCFSRYPDHMKRHDFFKSAMPEGYVQERTIYFKDDGTYKTRAEVKFEGDTLVNRIELKGID

SEQ ID NO: 72 - protein, 241 aa, Artificial sequence
  Recombinant Fluorescent Protein Sequence
MLEYNFNSHKVYITADKQNNGIKANFTIRHNVEDGSVQLADHYQQNTPIGDGPVLLPDDHYLSTQTILSKDLNEKRDHMVLLEYVTAAGITDASGGGSGGGSRKGEELFTGVVPILIELDGDVNGHKFFVRGEGEGDATIGKLSLKFICTTGKLPVPWPTLVTTLTYGVQCFSRYPDHMKRHDFFKSAMPEGYVQERTIYFKDDGTYKTRAEVKFEGDTLVNRIELKGIDFKEDGNILGHK

SEQ ID NO: 73 - protein, 241 aa, Artificial sequence
  Recombinant Fluorescent Protein Sequence
MNSHKVYITADKQNNGIKANFTIRHNVEDGSVQLADHYQQNTPIGDGPVLLPDDHYLSTQTILSKDLNEKRDHMVLLEYVTAAGITDASGGGSGGGSRKGEELFTGVVPILIELDGDVNGHKFFVRGEGEGDATIGKLSLKFICTTGKLPVPWPTLVTTLTYGVQCFSRYPDHMKRHDFFKSAMPEGYVQERTIYFKDDGTYKTRAEVKFEGDTLVNRIELKGIDFKEDGNILGHKLEYNF

SEQ ID NO: 74 - protein, 241 aa, Artificial sequence
  Recombinant Fluorescent Protein Sequence
MNNGIKANFTIRHNVEDGSVQLADHYQQNTPIGDGPVLLPDDHYLSTQTILSKDLNEKRDHMVLLEYVTAAGITDASGGGSGGGSRKGEELFTGVVPILIELDGDVNGHKFFVRGEGEGDATIGKLSLKFICTTGKLPVPWPTLVTTLTYGVQCFSRYPDHMKRHDFFKSAMPEGYVQERTIYFKDDGTYKTRAEVKFEGDTLVNRIELKGIDFKEDGNILGHKLEYNFNSHKVYITADKQ

SEQ ID NO: 75 - protein, 241 aa, Artificial sequence
  Recombinant Fluorescent Protein Sequence
MGSVQLADHYQQNTPIGDGPVLLPDDHYLSTQTILSKDLNEKRDHMVLLEYVTAAGITDASGGGSGGGSRKGEELFTGVVPILIELDGDVNGHKFFVRGEGEGDATIGKLSLKFICTTGKLPVPWPTLVTTLTYGVQCFSRYPDHMKRHDFFKSAMPEGYVQERTIYFKDDGTYKTRAEVKFEGDTLVNRIELKGIDFKEDGNILGHKLEYNFNSHKVYITADKQNNGIKANFTIRHNVED

SEQ ID NO: 76 - protein, 241 aa, Artificial sequence
  Recombinant Fluorescent Protein Sequence
MDGPVLLPDDHYLSTQTILSKDLNEKRDHMVLLEYVTAAGITDASGGGSGGGSRKGEELFTGVVPILIELDGDVNGHKFFVRGEGEGDATIGKLSLKFICTTGKLPVPWPTLVTTLTYGVQCFSRYPDHMKRHDFFKSAMPEGYVQERTIYFKDDGTYKTRAEVKFEGDTLVNRIELKGIDFKEDGNILGHKLEYNFNSHKVYITADKQNNGIKANFTIRHNVEDGSVQLADHYQQNTPIG

SEQ ID NO: 77 - protein, 241 aa, Artificial sequence
  Recombinant Fluorescent Protein Sequence
MPDDHYLSTQTILSKDLNEKRDHMVLLEYVTAAGITDASGGGSGGGSRKGEELFTGVVPILIELDGDVNGHKFFVRGEGEGDATIGKLSLKFICTTGKLPVPWPTLVTTLTYGVQCFSRYPDHMKRHDFFKSAMPEGYVQERTIYFKDDGTYKTRAEVKFEGDTLVNRIELKGIDFKEDGNILGHKLEYNFNSHKVYITADKQNNGIKANFTIRHNVEDGSVQLADHYQQNTPIGDGPVLL

SEQ ID NO: 78 - protein, 241 aa, Artificial sequence
  Recombinant Fluorescent Protein Sequence
MRDHMVLLEYVTAAGITDASGGGSGGGSRKGEELFTGVVPILIELDGDVNGHKFFVRGEGEGDATIGKLSLKFICTTGKLPVPWPTLVTTLTYGVQCFSRYPDHMKRHDFFKSAMPEGYVQERTIYFKDDGTYKTRAEVKFEGDTLVNRIELKGIDFKEDGNILGHKLEYNFNSHKVYITADKQNNGIKANFTIRHNVEDGSVQLADHYQQNTPIGDGPVLLPDDHYLSTQTILSKDLNEK

SEQ ID NO: 79 - DNA, 858 bp, Artificial sequence
  Recombinant Fluorescent Protein Sequence
atggacgaccattacctgtcgacacaaactatcctttcgaaagatctcaacggtggagggggttctggtggtggcggatctggtggcggttctgaaaagcgtgaccacatggtccttcttgagtatgtaactgctgctgggattacagatgctagcggtggcggttctggcggtggttctatgaggaaaggagaagaacttttcactggagtcgtcccaattcttattgaattagatggtgatgttaatgggcacaaattttttgtccgtggagagggtgaaggtgatgctacaatcggaaaactcagccttaaatttatttgcactactggaaaactacctgttccatggccaacacttgtcactactctgacctatggtgttcaatgcttttcccgttatccggatcacatgaaacggcatgactttttcaagagtgccatgcccgaaggttatgtacaggaacgcactatatatttcaaagatgacgggacctacaagacgcgtgctgaagtcaagtttgaaggtgatacccttgttaatcgtatcgagttaaagggtattgattttaaagaagatggaaacattcttggacacaaactcgagtacaactttaactcacacaaagtatacatcacggcagacaaacaaaataatggaatcaaagctaacttcacaattcgccacaacgttgaagatggttccgttcaactagcagaccattatcaacaaaatactccaattggcgatggccctgtccttttaccaggcggcggcagcggcgatgaagtggatggcggcagcggcagcgacgaccattacctgtcgtatcaaactatcctttcgaaagatctcaac

SEQ ID NO: 80 - protein, 286 aa, Artificial sequence
  Recombinant Fluorescent Protein Sequence
MDDHYLSTQTILSKDLNGGGGSGGGGSGGGSEKRDHMVLLEYVTAAGITDASGGGSGGGSMRKGEELFTGVVPILIELDGDVNGHKFFVRGEGEGDATIGKLSLKFICTTGKLPVPWPTLVTTLTYGVQCFSRYPDHMKRHDFFKSAMPEGYVQERTIYFKDDGTYKTRAEVKFEGDTLVNRIELKGIDFKEDGNILGHKLEYNFNSHKVYITADKQNNGIKANFTIRHNVEDGSVQLADHYQQNTPIGDGPVLLPGGGSGDEVDGGSGSDDHYLSYQTILSKDLN

SEQ ID NO: 81 - protein, 8 aa, Artificial sequence
  peptide linker
GGGSGGGS

SEQ ID NO: 82 - protein, 5 aa, Artificial sequence
  protease site
DEVDG

SEQ ID NO: 83 - protein, 236 aa, Artificial sequence
  Recombinant Fluorescent Protein Sequence
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 84 - protein, 225 aa, Artificial sequence
  Recombinant Fluorescent Protein Sequence
MRSSKNVIKEFMRFKVRMEGTVNGHEFEIEGEGEGRPYEGHNTVKLKVTKGGPLPFAWDILSPQFQYGSKVYVKHPADIPDYKKLSFPEGFKWERVMNFEDGGVVTVTQDSSLQDGCFIYKVKFIGVNFPSDGPVMQKKTMGWEASTERLYPRDGVLKGEIHKALKLKDGGHYLVEFKSIYMAKKPVQLPGYYYVDSKLDITSHNEDYTIVEQYERTEGRHHLFL

SEQ ID NO: 85 - protein, 71 aa, Artificial sequence
  Recombinant Fluorescent Protein Sequence
YTMGLPDNHYLSTQSVLSKDPNGTGGGSGGGSHMGGGSGSGGGSGGGSTSEKRDHMVLLEFVTAAGITGAS

SEQ ID NO: 86 - protein, 71 aa, Artificial sequence
  Recombinant Fluorescent Protein Sequence
YTMDLPDNHYLSTQTILLKDLNGTGVGSGGGSHMGGGSGSGGGSGGGSTSEKRDHMVLLEYVTAAGITDAS

SEQ ID NO: 87 - protein, 71 aa, Artificial sequence
  Recombinant Fluorescent Protein Sequence
YTMDLPDNHYLSTQTILLKDLNGTGVGSGGGSHMGGGSGSGGESGGGSTGEKRDHMVLLEYVTAAGITGAS

SEQ ID NO: 88 - protein, 71 aa, Artificial sequence; identical to SEQ ID NO: 86
  Recombinant Fluorescent Protein Sequence

SEQ ID NO: 89 - protein, 71 aa, Artificial sequence
  Recombinant Fluorescent Protein Sequence
YTMDLPDNHYLSTQTILLKDLNGTDVGSGGGSHMGGGSGSDGGSGGGSTGEKRDHMVLLEYVTAAGITGAS

SEQ ID NO: 90 - protein, 71 aa, Artificial sequence; identical to SEQ ID NO: 86
  Recombinant Fluorescent Protein Sequence

SEQ ID NO: 91 - protein, 71 aa, Artificial sequence
  Recombinant Fluorescent Protein Sequence
YTMDLPDNHYLSTQTILLKDLNGTGGGSGDGCHMDGGSGSGGGSGGGSTGEKRDHMVLLEYVTAAGITGAS

SEQ ID NO: 92 - protein, 12 aa, Artificial sequence
  peptide linker
GTGVGSGGGSHM

SEQ ID NO: 93 - protein, 33 aa, Artificial sequence
  peptide linker
GSGGGSGGGSTSEKRDHMVLLEYVTAAGITDAS

SEQ ID NO: 94 - protein, 32 aa, Artificial sequence
  Recombinant Fluorescent Protein Sequence
DVGGGGSEGGGSGGPGSGGEGSAGGGSAGGGS

SEQ ID NO: 95 - protein, 25 aa, Artificial sequence
  Recombinant Fluorescent Protein Sequence
GSGAGGSPGGGSGGSGSSASGGSTS

SEQ ID NO: 96 - protein, 66 aa, Aequorea victoria
SVQLADHYQQNTPIGDGPVLLPDNGSDLPDDHYLSTQTILSKDLNEKRDHMVLLEYVTAAGITDAS

SEQ ID NO: 97 - protein, 7 aa, Artificial sequence
  heptad repeat sequence
EVSALEK

SEQ ID NO: 98 - protein, 7 aa, Artificial sequence
  heptad repeat sequence
KVSALKE

SEQ ID NO: 99 - protein, 35 aa, Artificial sequence
  Coiled-Coil protein sequence
KVSALKENVSALKEKVSALTEKVSALKEKVSALKE

SEQ ID NO: 100 - protein, 35 aa, Artificial sequence
  Coiled-Coil protein sequence
KVSALENEVSALEKEVSVLEKEVSALEKEVRALEK

SEQ ID NO: 101 - protein, 35 aa, Artificial sequence; identical to SEQ ID NO: 100
  Coiled-Coil protein sequence

SEQ ID NO: 102 - protein, 35 aa, Artificial sequence; identical to SEQ ID NO: 100
  Coiled-Coil protein sequence

SEQ ID NO: 103 - DNA, 96 bp, Artificial Sequence
  Recombinant Fluorescent Protein Sequence
gacgttggtggtggcggatcagaaggaggcggtagcgggggccctggttcgggaggggaaggttctgctgggggagggagcgctggcggggggtct

SEQ ID NO: 104 - DNA, 75 bp, Artificial sequence
  Recombinant Fluorescent Protein Sequence
ggatccggcgctggcggaagccctgggggcgggagcggtggctctggttcttctgctagtggcggctcaacatct

(19) United States
(12) Patent Application Publication
(10) Pub. No.: US 2015/0099271 A1
(43) Pub. Date: Apr. 9, 2015

(54) FLUORESCENT PROTEINS, SPLIT FLUORESCENT PROTEINS, AND THEIR USES

(71) Applicant: LOS ALAMOS NATIONAL SECURITY, LLC, LOS ALAMOS, NM (US)

(72) Inventors: Geoffrey Waldo, Santa Fe, NM (US); Stephanie Cabantous, Toulouse Cedex (FR)

(73) Assignee: LOS ALAMOS NATIONAL SECURITY, LLC

(21) Appl. No.: 14/506,876

(22) Filed: Oct. 6, 2014

Related U.S. Application Data
(60) Provisional application No. 61/886,851, filed on Oct. 4, 2013.

(51) Int. Cl. C12Q 1/37; C07K 14/00

(52) U.S. Cl. CPC C12Q 1/37; C07K 14/00
USPC 435/23; 530/350; 536/23.1; 435/320.1; 435/252.33

(57) ABSTRACT

Disclosed herein are fluorescent proteins and Split-Fluorescent proteins (SFPs) including Split-Green Fluorescent Proteins, such as tripartite split-GFPs. Nucleic acid molecules encoding the fluorescent proteins and SFPs, as well as methods of using the fluorescent proteins and SFPs, are also disclosed. For example, methods of detecting protein-protein interactions are disclosed herein.

### CROSS REFERENCE TO RELATED APPLICATIONS

This application claims priority to U.S. Provisional Application No. 61/886,851, filed Oct. 4, 2013, which is incorporated by reference in its entirety.

### ACKNOWLEDGMENT OF GOVERNMENT SUPPORT

This invention was made with government support under Contract No. DE-AC52-06NA25396 awarded by the U.S. Department of Energy. The government has certain rights in the invention.

### FIELD

The present disclosure relates generally to compositions for improved fluorescent based protein-protein interaction and protein solubility detection methods.

### BACKGROUND

Fluorescent proteins such as Green Fluorescent Protein (GFP) from the Pacific Northwest jellyfish, Aequorea victoria, form a three dimensional structure including eleven anti-parallel outer beta strands and one inner alpha strand. Several natural and engineered GFP variants are known, including variants that exhibit altered fluorescent properties. However, there is a need for fluorescent proteins with altered excitation and emission profiles compared to GFP, particularly those with improved folding and solubility characteristics.

Split Fluorescent Proteins (SFPs) are composed of multiple fragments of the eleven anti-parallel outer β-strands and one inner α-strand of a fluorescent protein. Individually the fragments are not fluorescent, but, when complemented, form a functional fluorescent molecule. Typically, the SPF includes a first fragment known as a “SFP detector” that includes nine or ten contiguous β-strands and the α-strand of the fluorescent protein or a circular permutant thereof, and one or two separate fragments known as the “SFP tag(s)” that include the remaining β-strand or strands. Some tripartite SFP systems are known, which include three separate proteins that can form a fluorescent protein. For example, a tripartite split-Green Fluorescent Protein (split-GFP) system can include an SFP detector including GFP β-strands 1-9 (GFP1-9), a first SFP tag including GFP β-strand 10 (GFP10), and a third SFP tag including GFP β-strand 11 (GFP11). The GFP10 and GFP11 tags can be placed on unrelated polypeptide sequences and detected using the GFP1-9 detector. However, known split-GFP systems, particularly tripartite split-GFP systems, exhibit poor assembly characteristics and/or high background fluorescence.

### SUMMARY

Disclosed herein are polypeptides comprising novel SFP detectors and tags that have been engineered to provide improved assembly characteristics and lower background than known SFP detectors and tags. Novel methods are made possible by the improved SFP detectors and tags, and such methods are provided herein.

In some embodiments, an SFP detector including at least nine contiguous β-strands of a recombinant green fluorescent protein comprising the consensus amino acid sequence set forth as SEQ ID NO: 1 (GFP1-9 OPT consensus), or a circular permutant thereof, is provided. The SFP detector can complement with the remaining two β-strands of the SFP to form a fluorescent protein. In some embodiments, the SFP detector comprises or consists of the amino acid sequence set forth as SEQ ID NO: 1, wherein the detector can complement with a SFP 10 tag and a SFP 11 tag to form a fluorescent protein. In additional embodiments, the SFP detector comprises or consists of the amino acid sequence set forth as SEQ ID NO: 2 (GFP1-9 OPT WT), SEQ ID NO: 3 (GFP1-9 OPT1), or SEQ ID NO: 4 (GFP1-9 OPT2) wherein the SFP detector can complement with a SFP 10 tag and a SFP 11 tag to form a fluorescent protein.

The SFP detectors are useful, for example, in methods of detecting a protein-protein interaction between a first test polypeptide and a second test polypeptide. In some embodiments, the method comprises providing a GFP 1-9 detector comprising the amino acid sequence set forth as SEQ ID NO: 3 (GFP1-9 OPT1), a GFP 10 tag fused to the first test polypeptide, and a GFP11 tag fused to the second test polypeptide. If the first test polypeptide binds to the second test polypeptide, then the SFP 1-9 detector, the SFP 10 tag, and the SFP 11 tag can complement to form a fluorescent protein complex. Detecting the fluorescence of this protein complex detects the protein-protein interaction between the first and second test polypeptides.

Novel protease sensors based on the disclosed SFP polypeptides are also disclosed. In additional embodiments, a polypeptide comprising a fluorescent protein comprising the amino acid sequence set forth as SEQ ID NO: 8 (sfCherry) is provided. Nucleic acid molecules encoding these novel polypeptides, expression vectors including these nucleic acid molecules, and methods of their use are provided.

The foregoing and other features and advantages of this disclosure will become more apparent from the following detailed description of several embodiments which proceeds with reference to the accompanying figures.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1. Principle of the tripartite split-GFP complementation assay. β-strand 10 (GFP10) and β-strand 11 (GFP11) are fused to bait (A) and prey (B) proteins, respectively and the detector fragment GFP1-9 (β-strands 1-9) is added separately. When protein interaction occurs, GFP10 and GFP11 are tethered in relative close proximity to each other and then spontaneously associate with GFP1-9 fragment to form a complete GFP (as a complex of three proteins). If proteins A and B do not interact, GFP10 and GFP11 are not tethered in proximity to each other and entropy is too high to allow complementation with GFP1-9.

FIG. 2. Secondary structure diagram of GFP1-9 variants with corresponding mutations. Superfolder GFP1-9 (green dots), GFP1-9 M1 (yellow, also referred to as GFP1-9 WT), and GFP1-9 OPT1 (orange).

FIGS. 3A and 3B. Complementation of the tripartite split-GFP in vitro and in vivo. a) Complementation curves of GFP1-9 M1 (gray line) and GFP1-9 OPT1 (black line) with equimolar GFP10-sulfite reductase (SR)-GFP11 fusion protein (left) or GFP10-11 hairpin domain displayed on a permissive loop of a superfolder red fluorescent protein (right). b) In vivo solubility screen of 18 Pyrobaculum test proteins expressed in a “sandwich” configuration with N-terminal GFP10 and C-terminal GFP11 (GFP10-A-GFP11, A=protein of interest) from pTET-GFP10/11 plasmid assayed with either GFP1-10 (top) or GFP1-9 (bottom) expressed from a pET vector in BL21(DE3) E. coli. Fluorescence pictures of E. coli colonies on plates after 1½ h Antet induction followed by IPTG induction (sequential induction) (SEQ) or 3 h co-induction (CO). Legend indicates of the tagged proteins determined by SDS-PAGE (soluble (green), partially soluble (yellow), insoluble (red)).

FIGS. 4A-4D. Characterization of protein-protein interactions using coiled-coil heterodimerization a) Expression vectors for tripartite split-GFP interaction assays in E. coli: bicistronic pTET tetracycline inducible vector harboring GFP10 and GFP11 tags with cloning sites for test proteins; IPTG inducible pET vector (T7 promoter) for stable expression of GFP1-9 OPT1. b) Colony fluorescence on plates after Antet and IPTG co-induction driving expression of GFP10-K1/E1-GFP11 pair (K/E) (top) and GFP10-E1/E1-GFP11 (E1/E1) (bottom) with GFP1-9 OPT1 detection fragment. c) In vitro quantification of K1/E1 or E1/E1 interaction with increasing amount of E-GFP11 (0.4 nM up to 800 nM). Fluorescence values 1 hour after initiating complementation with GFP1-9 OPT1. d) Initial velocity rates of complementation reactions with various concentrations of GFP1-9 OPT1 detection reagent (0.5 μM up to 8 μM) in GFP10-K1/E1-GFP11 assay (1 μM of each tagged species).

FIGS. 5A-5D. Application of the tripartite split-GFP to study the rapamycin inducible FRB/FKBP interaction. a) GFP10 and GFP11 tags were fused to FRB and FKBP proteins. Rapamycin ligand binding brings both protein fusions into proximity, permitting GFP fluorescence reconstitution upon addition of GFP1-9 OPT1. b) Raw fluorescence progress curves for GFP1-9 OPT1 complementation with soluble extracts of GFP10-FRB and FKBP-GFP11 fusions in presence (+RAP) or absence (−RAP) of rapamycin (starting time marked by addition of rapamycin to initiate complementation) c) Fluorescence levels of GFP10-FRB and FKBP-GFP11 assayed at various concentrations (320, 160, and 40 nM in each) initiated by addition of rapamycin to 150 nM final concentration (black bar) and no rapamycin (gray bar). d) Rapamycin dose curve (0.6 to 300 nM) for FRB/FKPB binding in vitro measured as final fluorescence after 1 hour.

FIGS. 6A-6C. Monitoring complex formation and stability in E. coli. a) Analysis of YheNML heterotrimeric complex formation using split-GFP. GFP1-9 OPT1 complementation provides information on interaction of GFP10 and GFP11 tagged domains, while GFP1-10 binds to GFP11 tagged protein, thus providing expression and solubility levels of the GFP11 tagged protein or complex. b) E. coli polycistrons YheNML, YheML and YBJG/K were subcloned between GFP10 and GFP11 β-strands with extended linkers (125 and 130) into pTET GFP10/11 tet inducible plasmids. c) Split-GFP assay in E. coli cells expressing either GFP1-9 or GFP1-10. Sequential or co-induction was performed as previously described. Corresponding semi-quantitative measure of colony fluorescence using NIH Image (right).

FIGS. 7A and 7B. Visualization of complex formation in mammalian cells. a) Leucine zipper and Ku70/80 heterodimerization. Cells transiently expressing GFP1-9 OPT1 with GCN4 zipper (Z-11) (left panel) or Ku70-GFP11 (Ku70-11) (right panel) display no background fluorescence. Heterodimerization is visualized as fluorescence with interacting leucine zippers GFP10-Z and Z-GFP11 in CHO cells expressing GFP1-9 OPT1. Complementation of GFP11-Z with GFP1-10 confirms localization of the zipper alone (bottom). Ku70/80 complex formation is visualized in cell nuclei (right panel). Expression of one Ku component tagged with GFP11 is monitored with GFP1-10. FACS analysis of HEK 293_GFP1-9 cell lines transfected with corresponding constructs (24 h after transfection). Percentage of fluorescent cells (black bars); mean fluorescence intensity of the positive cell population (gray bars). Self-associating GFP10-Z-GFP11 domain is used as positive control of transfection and complementation with GFP1-9 (mean±SD; N=3). b) Rapamycin induced FRB/FKBP interaction in mammalian cells. Stable HEK 293 cells expressing GFP1-9 OPT1 were co-transfected with GFP10-FRB and FKBP-GFP11 constructs and stimulated with increasing concentrations of rapamycin (RAP) (0, 10, 20, 50 and 100 nM). Bipartite complementation of FKBP-11 and GFP1-10 is shown in the most left image; Green fluorescence at 488 nm excitation (GFP), DAPI nuclear staining (cyan). Scale bars=10 μm. Bottom panel: FACS quantification of rapamycin induced association with or without addition of competitive inhibitor FK-506 (1 μM). Right graph: addition of increasing concentrations of FK-506 (20 nM rapamycin; 0.01, 0.1 and 1 μM FK-506) (mean±SD; N=3).

FIGS. 8A and 8B. Solubility and in vitro complementation of folding reporter (FR) and superfolder (SF) GFP bimolecular fluorescence complementation (BiFC) fragments expressed individually in E. coli. A bipartite split-GFP system was previously developed to quantify protein solubility and follow protein localization in vivo. Considering that self-association between GFP11 and GFP1-10 makes this system unsuitable for protein-protein interaction studies, the behavior of BiFC fragments obtained by fragmentation of folding-reporter GFP4 (FR-GFP) and Superfolder GFP5 (SF-GFP) at permissive sites 156 and 1736 was assayed. a) FR and SF Split-GFP fragments were produced by splitting GFP at permissive 156 and 173 positions, as described in BiFC assays. Fragments (1-157) and (158-238), (1-173) and (174-238) were expressed from pET vectors in E. coli at 37° C. for 3 h. Soluble and insoluble fractions were quantified by SDS-PAGE. Small GFP fragments are mostly soluble whereas large fragments are largely insoluble. b) In vitro complementation assay combining soluble fractions from small split-GFP fragments with the soluble (S) or refolded (R) fractions of the corresponding large fragments (1-156) and (1-173). As observed with other fluorescent protein fragments used in refolding-based BiFC3, split-GFP fragments from refolded FR-GFP self-associate detectably (gray bars). Improved folding in SF-GFP fragments resulted in improved solubility, but also increased spontaneous fluorescence for all conditions (whether refolded or soluble extracts) (black bar).

FIG. 9. Helical coiled-coils as a model to study protein-protein interactions. E and K coils are constituted by five repeated heptads of the EVSALEK (SEQ ID NO: 97) or KVSALKE (SEQ ID NO: 98) motifs (gray boxes). The sequences shown (from top-to-bottom) are SEQ ID NOs: 99-102, respectively. Electrostatic interactions introduced in the first and sixth positions of the heptads direct preferential interaction, allowing interchain ionic attraction (K/E) or repulsion (E/E) (black arrows). Valine (V) and Leucine (L) residues maintain hydrophobic interactions between coils (bold). Directed evolution of wild-type sequences resulted in additional mutations that greatly increased solubility of K coil mutant 1 (K1) and E coil mutant 1 (E1) (underlined) while maintaining the specificity and tight binding of the wild type. E1 and K1 coils were used in the interaction studies.

FIGS. 10A and 10B. Vector map of pTET Bicistronic GFP10-IRBS-GFP11 vector. a) The pTET GFP10-IRBS-GFP11 vector is based on the previously designed expression vector (Cabantous and Waldo, Nat. Methods, 3, 845-854, 2006). It includes a bicistronic expression cassette under the control of the tet promoter. In one tagging module, N-terminal GFP10 is separated from protein A by a 12-mer linker (cloning sites NdeI/KpnI). An internal ribosome-binding site separates it from a second module comprising SpeI/BamHI cloning sites for protein B), a 12-mer linker, and the C-terminal GFP11 tag. b) Longer linkers of 30-mer (GFP10 tag, SEQ ID NO: 94 (protein) and SEQ ID NO: 103 (DNA)) and 25-mer (GFP11 tag, SEQ ID NO: 95 (protein) and SEQ ID NO: 104 (DNA)) were used for interaction assays of larger proteins (>50 kD). The pTET ColE ori GFP10-IRBS-GFP11 vector was used alone for in vitro assays or in combination with compatible pET p15 ori GFP1-9 OPT1 plasmid for studying interactions in E. coli.

FIGS. 11A-11C. Effect of split-point at the linker junction between GFP9 and GFP10 β-strands. a) To investigate the optimal split position between β-strands GFP9 and GFP10, GFP1-9 was designed with extended C-termini (red arrows) and corresponding GFP10 with truncated N-terminus (black arrows). Residues 168-233 of superfolder GFP are shown (SEQ ID NO: 96). b) Several truncated versions of GFP1-9 were expressed from pET vectors and tested for complementation in vivo with the non-interacting E/E or interacting K1/E1 coiled-coil pair expressed from pTET bicistron (GFP10-E1/E1-GFP11) or (GFP10-K1/E1-GFP11). c) Comparison of the output signal of the same bicistronic constructs as a function of N-terminal GFP10 truncations, GFP1-9 being kept fixed at amino acid position 193. Colony fluorescence after 2 h induction at 37° C. was compared for K1/E1 and E1/E1 coils pairs to evaluate the optimal split-point. Last permissive complementation was given for position 194, designating the final version of GFP10 used throughout the study. However, extension of C-termini of the GFP1-9 fragment (incrementally from amino-acid 193 to 197) had no effect on the fluorescence levels observed for interacting K1/E1 versus non-interacting E1/E1 coils.

FIGS. 12A and 12B. SDS-PAGE of soluble and Talon® purified fractions from 6HIS-tagged GFP10 and GFP11 fusion proteins with various linker sizes: 12mer, 25mer (indicated as 125) or 30mer (indicated as 130). To further characterize protein-protein interaction in vitro, K1 coil, E1 coil, or FRB were purified to 95% homogeneity. A 200 ml culture of BL21(DE3) cells expressing each construct was grown to OD600 nm˜0.5 in LB medium supplemented with 1 mM kanamycin, induced with 1 mM IPTG for 3 h at 37° C. Soluble extracts were purified on a 50% v/v slurry of metal affinity resin beads (Talon® resin, Clontech) in TNG buffer (100 mM Tris pH 8.0, 150 mM NaCl, 10% glycerol) according to standard procedures. Samples corresponding to the soluble and eluted fractions were resolved on a 4-20% gradient Criterion SDS-PAGE gel. Protein samples were stained using Gel Code Blue stain reagent (Pierce) and imaged using a GS-800 Calibrated Densitometer and quantified by Bio-Rad Protein Assay (Bio-Rad). a) Coiled-coils. Both C-6His (#2) and untagged (#3) versions of GFP10-K1 fusion expressed mostly in the soluble fraction. Flow-through fractions (FT) contained cellular proteins and untagged proteins. 90% pure proteins were recovered after one-step purification in the eluted fraction (EL). b) FKBP-GFP11 and GFP10-FRB fusions with different linker lengths. A sulfite reductase control protein (SR) sandwich with GFP10 and GFP11 tags was used as positive control of protein expression and complementation with GFP1-9.

FIG. 13. Amino acid sequences of GFP10 and GFP11 β-strands. In the sequences, underlined residues correspond to structural β-strand sequence from superfolder GFP, bold residues correspond to the additional point mutations introduced by mutagenesis, numbering corresponds to position in full-length superfolder GFP, and residues following amino acid 230 correspond to linker sequences.

FIG. 14. Amino-acid sequence alignment of evolved GFP10-11 proteins, including WT (SEQ ID NO:85), SM1 (SEQ ID NO: 86), SM2 (SEQ ID NO: 87), SM3 (SEQ ID NO: 88), SM4 (SEQ ID NO: 89), SM5 (SEQ ID NO: 90), and SM6 (SEQ ID NO: 91) GFP10-11 proteins. GFP10 and GFP11 are separated by a long flexible linker (wt, gray shade) that includes two cloning sites NdeI and BamHI. The entire cassette (GFP10-long flexible linker-GFP11) was shuffled and selected for optimized complementation with GFP1-9. Six optima (SM1-SM6) were isolated and sequenced. SM5 corresponds to the best variant, with mutations identified both in GFP10 (GFP10 M1) and only one mutation in GFP11 (GFP11 M4), thus differing from the previous GFP11M3 split-GFP domain identified with GFP1-10 complementation. Amino-acids corresponding to structural β-strands GFP10 and GFP11 are underlined in the wt sequence.

FIG. 15. Alignment of amino-acid sequences of GFP10 optima in HPS fusion. A partially soluble protein hexylose phosphate synthase (HPS) was cloned into the NdeI and BamHI cloning sites and the cassette was evolved, excluding HPS. Additional rounds of doped mutagenesis with oligonucleotides targeting GFP10 M1 were carried out, and resulted in mutant GFP10 M2. GFP11 sequence was unchanged. The GFP10 M1 construct includes (YT)-(GFP10 M1 tag, SEQ ID NO: 12)-(peptide linker, SEQ ID NO: 92)—(HPS)-(peptide linker, SEQ ID NO: 93)-(GFP11 M4 tag, SEQ ID NO: 15). The GFP10 M2 construct includes (YT)-(GFP10 M2 tag, SEQ ID NO: 13)-(peptide linker, SEQ ID NO: 92)-(HPS)-(peptide linker, SEQ ID NO: 93)-(GFP11 M4 tag, SEQ ID NO: 15).

FIGS. 16A-16E. (A) Alignment of five different GFP 1-9 detector sequences, including GFP1-9 OPT WT (SEQ ID NO: 2), GFP1-9 OPT 1 (SEQ ID NO: 3), and GFP1-9 OPT 2 (SEQ ID NO: 3). GFP 1-9 SF and GFP1-9 Original disclosure. Several consensus mutations such as S2R, T43S, K166T appeared during directed evolution of GFP1-9 WT against the GFP10-11 hairpin. Additional key mutations confer GFP1-9 OPT1 specific binding to sandwich 10-HPS-11: N39I, S99Y, K149N and K158N. (B) Comparison of GFP 1-9 versions from soluble fraction of E. coli liquid culture lysates, complemented with a 5-fold molar excess of the GFP 10-11 hairpin. The hairpin is fused to the superfolder cherry protein as disclosed in Example 2. For this experiment, the sfCherry acts as a passive carrier of the GFP 10-11 hairpin. The naming of the versions is same as in A. (C) Comparison of GFP 1-9 versions from soluble fraction of E. coli liquid culture lysates, complemented with a 5-fold molar excess 10-SR-11 (SR is sulfite reductase, a soluble carrier protein; e.g., as described in Int. Pub. No. WO2005074436, incorporated by reference herein in its entirety). (D) Comparison of GFP 1-9 versions from renatured inclusion bodies of E. coli liquid culture lysates, complemented with a 5-fold molar excess of GFP10-11 hairpin. The various GFP1-9 proteins have all been normalized to ca. 100 pmol in a 200 μl assay. The hairpin is fused to the superfolder cherry protein as disclosed in Example 2. The protein simply acts as a passive carrier of the GFP 10-11 hairpin. The naming of the versions is same as in A. (E) Comparison of GFP 1-9 versions from renatured inclusion bodies of E. coli liquid culture lysates, complemented with a 5-fold molar excess 10-SR-11. The various GFP 1-9 proteins have all been normalized to ca. 100 pmol in a 200 μl assay.

FIG. 17. Principle of the method of development of sfCherry: Insertion of GFP hairpin strands GFP10 and GFP11 into a permissive loop of a target protein, followed by reconstitution of the intact GFP by attachment of (association with) GFP1-9 (i.e. the GFP molecule missing the GFP10-11 hairpin).

FIGS. 18A-18C. (a) In vivo protein expression (left panel) and solubility screens (right panel) for sfCherry-GFP10-11 hairpin inserted at P52/G53 and D169/G170. Pictures were taken of the plates after 4 hours of co-induction (to monitor protein expression by GFP fluorescence) and after 2 hours of induction with anhydrotetracycline (AnTet) followed by 1 hour rest and 1 hour of induction of GFP1-9 (to monitor soluble protein by GFP fluorescence). Fluorescence from folded sfCherry was monitored using 550 nm excitation/610 nm emission (red fluorescence) and reconstituted GFP fluorescence was monitored using 488 nm excitation/520 nm emission (green fluorescence). Pictures are shown with 0.5 second exposure times for red fluorescence and 0.25 second exposure times for green fluorescence. (b) In vitro sensitivity characterization of sfCherry-GFP 10-11 complementation with GFP1-9. 20 μl aliquots containing 1.56 to 200 pmol of sfCherry-GFP10-11 hairpin were mixed with 180 μl aliquots containing 800 pmol of GFP1-9 to start the complementation. Arbitrary fluorescence units (A.U.) (c) Superimposition of scaled progress curves for complementation of 200, 100, 50, 25, 12.5, 6.25, 3.13 and 1.56 pmol samples. The curves can be well superimposed by linear scaling indicating that the shape of the progress curves does not depend on the concentration of the tagged protein or depletion of the pool of unbound GFP1-9 fragment (see Results).

FIGS. 19A and 19B. Three-dimensional structure of mCherry and sfCherry (a) Structure of mCherry (left, PDB code: 2H5Q) and sfCherry (right, see Example 2) showing the locations of sfCherry mutations and the corresponding residues in mCherry. (b) Region of mCherry and sfCherry close to residue 147 and 196 showing the hydrogen bonds formed between D196 and T147 and between D196 and R220 in the dimeric sfCherry structure (right) and the lack of corresponding hydrogen bonds between N196 with S147 or with R220 in monomeric mCherry structure (left).

FIGS. 20A and 20B. Three-dimensional structure of sfCherry-GFP10-11 hairpin complexed with GFP1-9. (a) The amino acid sequence of sfCherryl-9-GFP10-11 hairpin (SEQ ID NO: 8) is colored red for the sfCherry 1-9 component, blue for the GFP10-11 hairpin and cyan for the 3-residue linker (b) Structure of the sfCherry-GFP10-11 hairpin/GFP1-9 complex with the same color scheme used in the amino acid sequence. sfCherry forms dimers in the crystal through an interface involving the side chains of Threonine 106 (shown as spheres).

FIG. 21. A 2mFo-DFc sigmaA-weighted electron density map was calculated using a model that was constructed before any connections between sfCherry and the GFP10-11 hairpin had been built. The connections between the green arrows (not included in the phasing model) are residues 168 to 172 and residues 205 to 211. This unbiased map (contoured at 0.5 sigma (grey color) and at 1 sigma (blue color)) shows clear connections between sfCherry and the GFP10-11 hairpin that was inserted into sfCherry at an exposed loop.

FIG. 22. Stereo view showing an overlap of the two instances of the complex in the asymmetric unit. When the two GFP components (cyan, bottom) are superimposed, the sfCherry components in the two copies of the complex (yellow and pink) are seen to differ only by a small rotation.

FIGS. 23A and 23B. (A) Fluorescence images of E. coli cell colonies (left) and liquid cultures (right) expressing the best ferritin-Cherry fusion constructs obtained from third round of directed evolution compared to the starting ferritin-mCherry fusion construct. (B) Native gel showing sfCherry expressed alone runs as 50% monomer: 50% dimer at concentration of ˜10 mg/ml

FIGS. 24A and 24B. (A) In vitro characterization of sfCherry-GFP10-11 complementation with GFP1-9 or GFP1-10. 20 μl of sfCherry-GFP10-11 hairpin at concentration of 200 pmol per 20 μl was mixed with 180 μl aliquots containing 800 pmol of GFP1-9 or GFP1-10. (B) Sequence alignment of sfCherry (SEQ ID NO: 8), mCherry (SEQ ID NO: 83) and DsRed (SEQ ID NO: 84). Mutations found in sfCherry are highlighted in yellow, T106 is highlighted in cyan and L125 is highlighted in magenta.

FIGS. 25A-25D. Fluorescence images of sfCherry (labeled #1), sfCherry-GFP10-11 hairpin (labeled #2), sfCherry-GFP10-11 hairpin complemented with GFP1-9 (labeled #3) under (A) 550 nm excitation, 610 nm emission, (B) 488 nm excitation, 520 nm emission and (C) white light. Images of sfCherry (#1) and sfCherry-GFP10-11 hairpin complemented with GFP1-9 (#3) crystals are shown in (D).

FIGS. 26A and 26B. (A) Dimer interface for the reconstituted GFP in sfCherry-GFP10-11 hairpin/GFP1-9 complex structure with involving residues labeled in balls and sticks. (B) Superimposition of sfCherry dimers in its own structure (yellow color) with sfCherry dimers (red color) formed from lattice symmetry related sfCherry-GFP10-11 hairpin/GFP1-9 complex molecules.

FIG. 27. Schematic diagram illustrating circular permutants of GFP, and the optimizing amino acid substitutions for the GFP1-9 WT, OPT1, and OPT2 constructs described herein. Color coding for the amino acid substitutions is as shown in FIG. 2. SFP detectors including nine contiguous β-strands of GFP are illustrated. The circled number indicates the start point (N-terminus) of the circular permutant SFP detector, which are names as follows: (1) GFP2-10, (2) GFP3-11, (3) GFP4-1a, (4) GFP4-1b, (5) GFP5-2, (6) GFP6-3, (7) GFP7-4a, (8) GFP7-4b, (9) GFP7-4c, (10) GFP8-5, (11) GFP9-6, (12) GFP10-7a, (13) GFP10-7b, and (14) GFP11-8. For detectors including the 11 and 1 β-strands, these strands were joined by a heterologous linker as indicated. The circular permutant GFP illustrated in the figure starts at breakpoint indicated by circled 11, with a new amino acid start is 173. Translation proceeds through amino acid 233, then through the linker GGGSGGGS SEQ ID NO: 81) connecting strand 11 to strand 1. Translation continues through to amino acid 172 of the original sequence, followed by a stop codon.

FIGS. 28A and 28B. Alignment of the polypeptide sequence of a caspase-3 activity sensor (SEQ ID NO: 80), and a nucleic acid sequence encoding the sensor (SEQ ID NO: 79).

### SEQUENCE LISTING

The nucleic and amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. The Sequence Listing is submitted as an ASCII text file in the form of the file named “Sequence.txt” (˜24 kb), which was created on Sep. 29, 2014 which is incorporated by reference herein. In the accompanying sequence listing:

SEQ ID NO: 1 is a consensus polypeptide sequence of a GFP1-9 SFP-detector

SEQ ID NO: 2 is the polypeptide sequence of the GFP1-9 WT SFP detector.

SEQ ID NO: 3 is the polypeptide sequence of the GFP1-9 OPT1 SFP detector.

SEQ ID NO: 4 is the polypeptide sequence of the GFP1-9 OPT2 SFP detector.

SEQ ID NOs: 5-7 are exemplary nucleotide sequences encoding the GFP1-9 WT, GFP1-9 OPT1, and GFP1-9 OPT2 SFP detectors, respectively.

SEQ ID NO: 8 is the amino acid sequence of the sfCherry protein.

SEQ ID NOs: 9 and 10 are exemplary nucleic acid sequences encoding the sfCherry protein.

SEQ ID NOs: 11-15 are the amino acid sequences of exemplary SFP tags.

SEQ ID NOs: 16-20 are the nucleotide sequences of oligonucleotide primers.

SEQ ID NOs: 21-23 are the amino acid sequences of peptide linkers.

SEQ ID NOs: 24 and 25 are the amino acid sequences of soluble peptide linkers.

SEQ ID NO: 26 is the amino acid sequence of a SFP tag including β-strands 10 and 11.

SEQ ID NOs: 27-50 are the nucleotide sequences of oligonucleotide primers.

SEQ ID NOs: 51-64 are exemplary nucleic acid sequences encoding SFP detectors.

SEQ ID NOs: 65-78 are the amino acid sequences of SFP detectors.

SEQ ID NO: 79 is an exemplary nucleic acid sequence encoding a caspase-3 protease sensor.

SEQ ID NO: 80 is the amino acid sequence of a caspase-3 protease sensor.

SEQ ID NO: 81 is the amino acid sequence of a peptide linker.

SEQ ID NO: 82 is the amino acid sequence of a caspase-3 cleavage site.

SEQ ID NO: 83 is the amino acid sequence of mCherry.

SEQ ID NO: 84 is the amino acid sequence of DsRed.

SEQ ID NOs: 85-91 are the amino acid sequences of GFP10-11 proteins.

SEQ ID NOs: 92 and 93 are the amino acid sequences of peptide linkers.

SEQ ID NOs: 94 and 95 are the amino acid sequences of polypeptide linkers including the GFP 10 or GFP11 tags, respectively.

SEQ ID NO: 96 is amino acids 168-233 of superfolder GFP.

SEQ ID NOs: 97 and 98 are heptad repeat motif amino acid sequences.

SEQ ID NOs: 99-102 are the amino acid sequences of coiled-coil proteins.

SEQ ID NOs:103 and 104 are exemplary nucleic acid sequences encoding polypeptide linkers including the GFP 10 or GFP11 tags, respectively.

### DETAILED DESCRIPTION

### I. Summary of Terms

Unless otherwise noted, technical terms are used according to conventional usage. Definitions of common terms in molecular biology can be found in Benjamin Lewin, Genes VII, published by Oxford University Press, 1999; Kendrew et al. (eds.), The Encyclopedia of Molecular Biology, published by Blackwell Science Ltd., 1994; and Robert A. Meyers (ed.), Molecular Biology and Biotechnology: a Comprehensive Desk Reference, published by VCH Publishers, Inc., 1995; and other similar references.

As used herein, the singular forms “a,” “an,” and “the,” refer to both the singular as well as plural, unless the context clearly indicates otherwise. For example, the term “an antigen” includes single or plural antigens and can be considered equivalent to the phrase “at least one antigen.” As used herein, the term “comprises” means “includes.” Thus, “comprising an antigen” means “including an antigen” without excluding other elements. It is further to be understood that any and all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particular suitable methods and materials are described below. In case of conflict, the present specification, including explanations of terms, will control. In addition, the materials, methods, and examples are illustrative only and not intended to be limiting. To facilitate review of the various embodiments, the following explanations of terms are provided:

Binding: A specific interaction between two molecules. For example, binding can occur between a fragments of a split fluorescent protein (e.g., a GFP1-9 detector, GFP10 tag and a GFP11 tag), or between a receptor and a particular ligand. Binding can be specific and selective, so that one molecule is bound preferentially when compared to another molecule. In one example, specific binding is identified by a disassociation constant (K_{d}) of an agent for a particular protein or class of proteins, compared to the K_{d} for one or more other proteins.

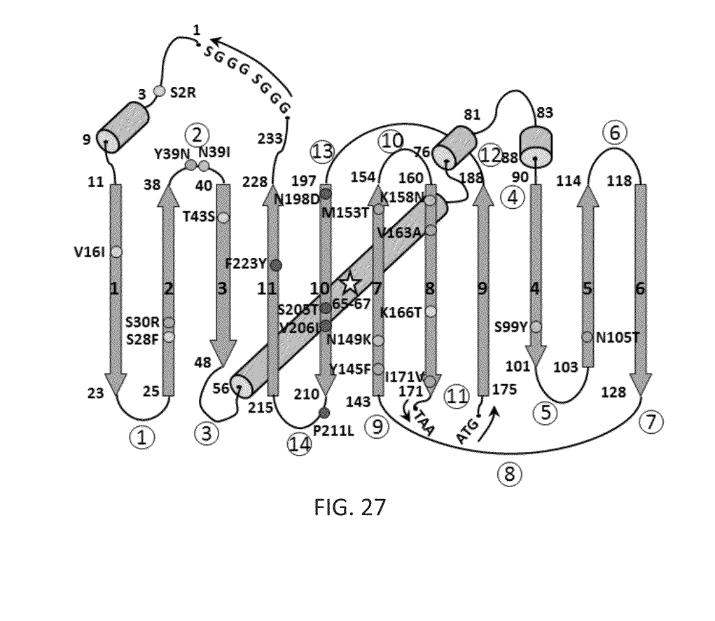

Circular Permutant: A recombinant protein in which the connections between different regions of a native protein tertiary structure are modified, so that the relative order of different regions in the primary sequence is altered, but the placement of the regions in the tertiary structure is preserved. As illustrated in FIG. 27, one example of a circular permutant GFP includes a peptide linker joining the native N- and C-termini, rearrangement of the native β-strands such that the strand 9 is at the N-terminus of the circular permutant, and strand 8 is at the C-terminus, yielding a β-strand tertiary structure including, from N- to C-terminus, β-strands 9-10-11-linker-1-2-3-4-5-6-7-8.

Conservative variants: “Conservative” amino acid substitutions are those substitutions that do not substantially affect or decrease a function of a protein, such as the fluorescent properties of a fluorescent protein. The term conservative variation also includes the use of a substituted amino acid in place of an unsubstituted parent amino acid. Furthermore, one of ordinary skill will recognize that individual substitutions, deletions or additions which alter, add or delete a single amino acid or a small percentage of amino acids (for instance less than 5%, in some embodiments less than 1%) in an encoded sequence are conservative variations where the alterations result in the substitution of an amino acid with a chemically similar amino acid.

Conservative amino acid substitution tables providing functionally similar amino acids are well known to one of ordinary skill in the art. The following six groups are examples of amino acids that are considered to be conservative substitutions for one another:

1) Alanine (A), Serine (S), Threonine (T);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V); and

6) Phenylalanine (F), Tyrosine (Y), Tryptophan (W).

Non-conservative substitutions are those that alter a function of the protein, such as the fluorescent properties of a fluorescent protein. For instance, if an amino acid residue is essential for a function of the protein, even an otherwise conservative substitution may disrupt that activity. Thus, a conservative substitution does not alter the basic function of a protein of interest.

Expression: The process by which the coded information of a gene is converted into an operational, non-operational, or structural part of a cell, such as the synthesis of a protein.

Fluorescent protein: A protein or protein complex that has the ability to emit light of a particular wavelength (emission wavelength) when exposed to light of another wavelength (excitation wavelength). Non-limiting examples of fluorescent proteins include the green fluorescent protein (GFP; see, for instance, GenBank Accession Number M62654) from the Pacific Northwest jellyfish, Aequorea victoria and natural and engineered variants thereof (see, for instance, U.S. Pat. Nos. 5,804,387; 6,090,919; 6,096,865; 6,054,321; 5,625,048; 5,874,304; 5,777,079; 5,968,750; 6,020,192; and 6,146,826; and published international patent application WO 99/64592). Other examples include Split-GFP, Split-YFP (described herein), Split-CFP (described herein) and Split-GFP variants, folding variants of GFP (e.g., more soluble versions, superfolder versions), spectral variants of GFP which have a different fluorescence spectrum (e.g., YFP, CFP), and GFP-like fluorescent proteins (e.g., DsRed; and DsRed variants, including DsRed1, DsRed2 (see, e.g., Matz et al., Nat. Biotechnol., 17:969-973, 1999). Fluorescent proteins with distinct excitation and emission properties are familiar to the skilled artisan; for example, functional GFPs, CFPs and YFPs comprise distinct excitation and emission properties. (see. e.g., Tsien, Annu. Rev. Biochem., 67:509-544, 1998.)

Fused: Linkage by covalent bonding. In some embodiments, “fused” refers to making two polypeptides into one contiguous polypeptide molecule by recombinant means.

Green Fluorescent Protein (GFP): A fluorescent protein from the Pacific Northwest jellyfish, Aequorea Victoria (see, e.g., GenBank Accession No. M62654, incorporated by reference herein), that forms a three dimensional structure including eleven anti-parallel outer beta strands and one inner alpha strand. Several natural and recombinant GFP variants are known, including variants that exhibit altered fluorescent properties.

Host Cell or Recombinant Host Cell: A cell that has been genetically altered, or is capable of being genetically altered by introduction of an exogenous polynucleotide, such as a recombinant plasmid or vector. Typically, a host cell is a cell in which a vector can be propagated and its DNA expressed. The cell may be prokaryotic or eukaryotic. For example, the host cell may be a bacteria cell, including an E. coli cell. “Host cell” also includes a colony of cells, for example, a colony of E. coli cells. Thus, “contacting a host cell” and “incubating a host cell” include contacting a colony of host cells or incubating a colony of host cells. The term also includes any progeny of the subject host cell. It is understood that all progeny may not be identical to the parental cell since there may be mutations that occur during replication. However, such progeny are included when the term “host cell” is used. A host cell encompasses material inside the outermost cell membrane, the outermost cell membrane itself and material fused or attached to the outermost cell membrane. In the case of a cell having a cell wall, the outermost cell membrane is the cell wall. Thus, the phase “within a host cell” includes material inside the outermost cell membrane, the outermost cell membrane itself and material fused or attached to the outermost cell membrane.

Isolated: A biological component (such as a host cell, nucleic acid molecule or polypeptide) that has been substantially separated or purified away from other biological components in the medium, cell or organism in which the component occurs. The term isolated does not require absolute purity. Nucleic acids and proteins that have been isolated include nucleic acids and proteins purified by standard purification methods. The term also embraces nucleic acids and proteins prepared by recombinant expression in a host cell, as well as chemically synthesized nucleic acids.

Multiple cloning site (MCS): A region of DNA containing a series of restriction enzyme recognition sequences. Typically, the restriction sites are only present once in the MCS. Vectors and plasmids used for cloning and expression typically contain a MCS to facilitate insertion of a heterologous nucleic acid sequence, such as the coding sequence of a gene of interest. In some embodiments, a MCS comprising at least two, at least three, at least four, at least five or at least six restriction enzyme recognition sites. The restriction sites may be immediately adjacent, they may overlap, there may be one or more nucleic acids between the sites, or any combination thereof.

Nucleic acid: A polymeric form of nucleotides, which may include both sense and anti-sense strands of RNA, cDNA, genomic DNA, and synthetic forms and mixed polymers thereof. A nucleotide refers to a ribonucleotide, deoxynucleotide or a modified form of either type of nucleotide. The phrase nucleic acid molecule as used herein is synonymous with nucleic acid and polynucleotide. A nucleic acid molecule is usually at least six bases in length, unless otherwise specified. The term includes single- and double-stranded forms. The term includes both linear and circular (plasmid) forms. A polynucleotide may include either or both naturally occurring and modified nucleotides linked together by naturally occurring nucleotide linkages and/or non-naturally occurring chemical bonds and/or linkers.

Nucleic acid molecules may be modified chemically or biochemically or may contain non-natural or derivatized nucleotide bases, as will be readily appreciated by those of skill in the art. Such modifications include, for example, labels, methylation, substitution of one or more of the naturally occurring nucleotides with an analog, internucleotide modifications, such as uncharged linkages (for example, methyl phosphonates, phosphotriesters, phosphoramidates, carbamates, etc.), charged linkages (for example, phosphorothioates, phosphorodithioates, etc.), pendent moieties (for example, polypeptides), intercalators (for example, acridine, psoralen, etc.), chelators, alkylators, and modified linkages (for example, alpha anomeric nucleic acids, etc.). The term nucleic acid molecule also includes any topological conformation, including single-stranded, double-stranded, partially duplexed, triplexed, hairpinned, circular and padlocked conformations. Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule

Unless specified otherwise, the left hand end of a polynucleotide sequence written in the sense orientation is the 5′-end and the right hand end of the sequence is the 3′-end. In addition, the left hand direction of a polynucleotide sequence written in the sense orientation is referred to as the 5′-direction, while the right hand direction of the polynucleotide sequence is referred to as the 3′-direction. Further, unless otherwise indicated, each nucleotide sequence is set forth herein as a sequence of deoxyribonucleotides. It is intended, however, that the given sequence be interpreted as would be appropriate to the polynucleotide composition: for example, if the isolated nucleic acid is composed of RNA, the given sequence intends ribonucleotides, with uridine substituted for thymidine.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions, in the same reading frame.

Promoter: A promoter is an array of nucleic acid control sequences which direct transcription of a nucleic acid. A promoter includes necessary nucleic acid sequences near the start site of transcription. A promoter also optionally includes distal enhancer or repressor elements. A “constitutive promoter” is a promoter that is continuously active and is not subject to regulation by external signals or molecules. In contrast, the activity of an “inducible promoter” is regulated by an external signal or molecule (for example, a transcription factor).

Protein or Polypeptide: A polymer of amino acid residues, including amino acid polymers in which one or more amino acid residue is an artificial chemical mimetic of a corresponding naturally occurring amino acid, as well as to naturally occurring amino acid polymers and non-naturally occurring amino acid polymer. Multiple polymers of amino acids binding to each other are a protein complex. Protein and polypeptide may be used interchangeably throughout this application and mean at least two covalently attached amino acids, which includes proteins, polypeptides, oligopeptides and peptides. Methods of manufacturing polypeptides are known to the skilled artisan and further described herein. For example, the polypeptides disclosed herein may be produced in cell-free systems, or in prokaryotic or eukaryotic cells.

Recombinant: A recombinant nucleic acid is one that has a sequence that is not naturally occurring or has a sequence that is made by an artificial combination of two otherwise separated segments of sequence. This artificial combination can be accomplished by chemical synthesis or, more commonly, by the artificial manipulation of isolated segments of nucleic acids, for example, by genetic engineering techniques. A recombinant protein is one that has a sequence that is not naturally occurring or has a sequence that is made by an artificial combination of two otherwise separated segments of sequence. In several embodiments, a recombinant protein is encoded by a heterologous (for example, recombinant) nucleic acid that has been introduced into a host cell, such as a bacterial or eukaryotic cell. The nucleic acid can be introduced, for example, on an expression vector having signals capable of expressing the protein encoded by the introduced nucleic acid or the nucleic acid can be integrated into the host cell chromosome.

Sequence identity/similarity: The primary sequence similarity between two nucleic acid molecules, or two amino acid molecules, is expressed in terms of the similarity between the sequences, otherwise referred to as sequence identity. Sequence identity is frequently measured in terms of percentage identity (or similarity or homology); the higher the percentage, the more similar are the two sequences.

Methods of alignment of sequences for comparison are well known in the art. Various programs and alignment algorithms are described in: Smith and Waterman, Adv. Appl. Math., 2:482, 1981; Needleman and Wunsch, J. Mol. Biol., 48:443, 1970; Pearson and Lipman, Proc. Natl. Acad. Sci. U.S.A., 85:2444, 1988; Higgins and Sharp, Gene, 73:237-244, 1988; Higgins and Sharp, CABIOS, 5:151-153, 1989; Corpet et al. Nuc. Acids Res., 16:10881-10890, 1988; Huang et al., Comp. Appls Biosci., 8:155-165, 1992; and Pearson et al., Meth. Mol. Biol., 24:307-31, 1994). Altschul et al., Nat. Genet., 6:119-129, 1994, presents a detailed consideration of sequence alignment methods and homology calculations.

By way of example, the alignment tools ALIGN (Myers and Miller, CABIOS 4:11-17, 1989) or LFASTA (Pearson and Lipman, 1988) may be used to perform sequence comparisons (Internet Program® 1996, W. R. Pearson and the University of Virginia, fasta20u63 version 2.0u63, release date December 1996). ALIGN compares entire sequences against one another, while LFASTA compares regions of local similarity. These alignment tools and their respective tutorials are available on the Internet at the NCSA Website, for instance. Alternatively, for comparisons of amino acid sequences of greater than about 30 amino acids, the Blast 2 sequences function can be employed using the default BLOSUM62 matrix set to default parameters, (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties). The BLAST sequence comparison system is available, for instance, from the NCBI web site; see also Altschul et al., J. Mol. Biol., 215:403-410, 1990; Gish. & States, Nature Genet., 3:266-272, 1993; Madden et al. Meth. Enzymol., 266:131-141, 1996; Altschul et al., Nucleic Acids Res., 25:3389-3402, 1997; and Zhang & Madden, Genome Res., 7:649-656, 1997.

Protein orthologues are typically characterized by possession of greater than 75% sequence identity counted over the full-length alignment with the amino acid sequence of a specific reference protein, using ALIGN set to default parameters. Proteins with even greater similarity to a reference sequence will show increasing percentage identities when assessed by this method, such as at least 80%, at least 85%, at least 90%, at least 92%, at least 95%, or at least 98% sequence identity. In addition, sequence identity can be compared over the full length of particular domains of the disclosed peptides.

When significantly less than the entire sequence is being compared for sequence identity, homologous sequences will typically possess at least 80% sequence identity over short windows of 10-20 amino acids, and may possess sequence identities of at least 85%, at least 90%, at least 95%, or at least 99%. Sequence identity over such short windows can be determined using LFASTA; methods are described at the NCSA Website; also, direct manual comparison of such sequences is a viable if somewhat tedious option.

One of skill in the art will appreciate that the sequence identity ranges provided herein are provided for guidance only; it is entirely possible that strongly significant homologs could be obtained that fall outside of the ranges provided.

The similarity/identity between two nucleic acid sequences can be determined essentially as described above for amino acid sequences. Nucleic acid sequences that do not show a high degree of identity may nevertheless encode similar amino acid sequences, due to the degeneracy of the genetic code. It is understood that changes in nucleic acid sequence can be made using this degeneracy to produce multiple nucleic acid sequences that each encode substantially the same protein.

Specifically hybridizable and specifically complementary are terms that indicate a sufficient degree of complementarity such that stable and specific binding occurs between the oligonucleotide (or it's analog) and the DNA or RNA target. The oligonucleotide or oligonucleotide analog need not be 100% complementary to its target sequence to be specifically hybridizable. An oligonucleotide or analog is specifically hybridizable when binding of the oligonucleotide or analog to the target DNA or RNA molecule interferes with the normal function of the target DNA or RNA, and there is a sufficient degree of complementarity to avoid non-specific binding of the oligonucleotide or analog to non-target sequences under conditions where specific binding is desired, for example under physiological conditions in the case of in vivo assays or systems. Such binding is referred to as specific hybridization.

Split-Fluorescent Protein (SFP): A protein complex composed of two or more protein fragments that individually are not fluorescent, but, when formed into a complex, result in a functional (that is, fluorescing) fluorescent protein complex. Split-GFP is an exemplary SFP. Individual protein fragments of a SFP are known as complementing fragments or complementary fragments. Complementing fragments which will spontaneously assemble into a functional fluorescent protein complex are known as self-complementing, self-assembling, or spontaneously-associating complementing fragments. A complemented split fluorescent protein complex is a protein complex comprising all the complementing fragments of a SFP necessary for the SFP to be active (i.e., fluorescent). Some examples of SFP fragments include SFP tags and SFP detectors, which are further described herein.

Complementary SFP fragments can be derived from the three dimensional structure of GFP, which includes eleven anti-parallel outer beta strands and one inner alpha strand (see e.g., the GFP structure deposited as PDB No. 1EMA, and Ormo et al., Science, 273:1392-5, 1996, and Yang et al., Nat. Biotechnol., 14:1246-51, 1996.) An SFP tag corresponds to one or two of the eleven beta-strands of the GFP molecule (e.g., GFP10 or GFP11 or both), and a SFP detector corresponds to the remaining nine or ten β-strands and the α-strand of the GFP (e.g., GFP1-9). A SFP10 tag (e.g., a GFP10 tag) includes β-strand 10 of an eleven stranded fluorescent protein β-barrel, and a SFP11 tag (e.g., GFP11 tag) includes β-strand 11 of an eleven stranded fluorescent protein β-barrel. Other combinations of fragments are also possible, for example, as disclosed herein and in U.S. Pat. App. Pub. No. 2005/0221343 and PCT Pub. No. WO/2005/074436. Certain SFPs are further disclosed herein, including examples of a tripartite SFP system.

Split-GFP: A SFP composed of multiple self-assembling protein fragments (e.g., a SFP detector and an SFP tag) that individually are not fluorescent, but, when complemented, form a functional (i.e., fluorescent) GFP. See, e.g., U.S. Pat. App. Pub. No. 2005/0221343 and Int. Pat. App. Pub. No. WO/2005/074436; and Cabantous et al., Nat. Biotechnol., 23:102-107, 2005; Cabantous and Waldo, Nat. Methods, 3:845-854, 2006. A functional (that is, fluorescing) GFP is a fluorescent protein or protein complex that can be distinguished based on excitation and emission properties. For example, typically, a functional GFP is a fluorescent protein or protein complex with predominantly green fluorescent characteristics (e.g., an emission peak of approximately 510 nm and an excitation peak of approximately 488 nm).

Several split-GFP fragments and variants thereof are known; see, e.g., U.S. Pat. App. Pub. No. 2005/0221343, which is incorporated by reference herein in its entirety. Novel split-GFP fragments are provided herein, including a novel Split-GFP1-9 detector, which can complement with GFP10 and GFP11 tags to form the complete GFP fluorophore. GFP1-9 corresponds to GFP beta strands 1-9, GFP10 corresponds to beta strand 10, and GFP11 corresponds to beta strand 11.

Subcellular compartment: A portion or section of a cell that is less than the whole cell. For example, a subcellular compartment may be an organelle within a cell, a membrane within a cell or an area surrounding a particular structure of a cell. Examples of subcellular compartments within eukaryotic cells include cytoplasm, nucleus, mitochondria, Golgi apparatus, endoplasmic reticulum (ER), peroxisome, lysosomes, endosomes (early, intermediate, late, etc.), vacuoles, cytoskeleton, nucleoplasm, nucleolus, nuclear matrix and ribosomes. In some examples, a subcellular compartment can be defines by proximity to a particular location within a cell, for example, the post-synaptic density of a neuron. See, e.g., Alberts et al., Molecular Biology of the Cell, 5^{th} edition, New York, Garland Science, 2005.

Subcellular localization: The location of a molecule in relation to a subcellular compartment.

Subcellular localization element: A molecule capable of directing a protein of interest to a particular subcellular compartment when the molecule is in contact with the protein. Non-limiting examples include protein, DNA, RNA, lipid, carbohydrate and small molecules capable of directing a protein to a subcellular compartment when in contact with the protein. The skilled artisan is familiar with molecules capable of directing a protein of interest to a particular subcellular compartment, and such molecules are further described herein. In some examples, the subcellular localization element is a mannose-6-phosphate moiety. In other examples, the subcellular localization element is a tag, which directs a heterologous protein that it is fused to a particular subcellular compartment. Exemplary subcellular localization elements and their use are provided, for example, in U.S. Pub. No. 2012/0282643, which is incorporated by reference herein in its entirety.

Vector: A nucleic acid molecule allowing insertion of foreign nucleic acid without disrupting the ability of the vector to replicate and/or integrate in a host cell. A vector can include nucleic acid sequences that permit it to replicate in a host cell, such as an origin of replication. A vector can also include one or more selectable marker genes and other genetic elements known in the art. An integrating vector is capable of integrating itself into a host nucleic acid. An expression vector is a vector that contains the necessary regulatory sequences to allow transcription and translation of inserted gene or genes.

### II. Description of Several Embodiments

### A. Split-GFP

SFPs are a protein complex composed of two or more protein fragments that individually are not fluorescent, but, when formed into a complex, result in a functional (that is, fluorescing) fluorescent molecule. Complementary sets of such fragments, are also known as a SFP system, and typically include a SFP detector (comprising 9-10 strands of an 11 β-barrel fluorescent protein) and one or two SFP tags (comprising the remaining strands of the fluorescent protein). The SFP detector complements with the heterologous SFP tag (or tags) to form a functional (that is, fluorescing) fluorescent protein. Thus, an SFP tag and the complementary SFP detector are two complementing fragments of a SFP. Novel GFP1-9 detectors (comprising 9 β-strands of an 11-β-barrel fluorescent protein) are disclosed that have been optimized to include novel properties unavailable with previously known GFP1-9 detectors, and which complement with known SFP tags, such as GFP10 and GFP11 tags.

Construction of a test protein fused to a SFP tag or SFP detector is typically accomplished via cloning of the nucleic acid encoding the test protein into a nucleic acid construct encoding the SFP tag or SFP detector. SFPs, SFP systems, a number of specifically engineered tag and detector fragments of a SFP, as well as DNA constructs and vectors use thereof are disclosed herein and known to the skilled artisan. See, e.g., U.S. Pat. App. Pub. No. 2005/0221343; Int. Pat. App. Pub. No. WO/2005/074436; Cabantous et al., Nat. Biotechnol., 23:102-107, 2005; Cabantous and Waldo, Nat. Methods, 3:845-854, 2006.) Typically, the SFPs include two SFP fragments, such as a SFP tag (typically corresponding to GFP11) and a SFP detector (typically corresponding to GFP1-10). Other SFPs are disclosed herein.

Polypeptides comprising Split-GFP fragments are known to the skilled artisan and further described herein. See, e.g., U.S. Pat. App. Pub. No. 2005/0221343 and Int. Pat. App. Pub. No. WO/2005/074436, and Cabantous et al., Nat. Biotechnol., 23:102-107, 2005; Cabantous and Waldo, Nat. Methods, 3:845-854, 2006. For example, in some embodiments, GFP1-10 OPT (SEQ ID NO: 4) may be used as a Split-GFP1-10 fragment. A corresponding SFP tag, for example, GFP11 M3 (SEQ ID NO: 16) may be used as the complementing Split-GFP11 fragment. Other variations are also available; see, e.g., U.S. Pat. App. Pub. No. 2005/0221343. The polypeptides comprising complementing Split-GFP fragments disclosed herein will form a functional GFP molecule when complemented.

### B. Tripartite Split Fluorescent Proteins

Disclosed herein are polypeptides comprising SFP detectors, including tripartite SFP detectors. In some embodiments the SFP detector includes at least nine contiguous β-strands of a recombinant green fluorescent protein comprising the consensus amino acid sequence set forth as SEQ ID NO: 1 or a circular permutant thereof. The SFP detector can complement with the remaining two β-strands of the SFP to form a fluorescent protein.

For example, in some embodiments a GFP 1-9 detector is provided, which includes β-strands 1-9 of a β-barrel fluorescent protein, and which is capable of complementation with strands 10 and 11 of the β-barrel fluorescent protein to form a functional (that is, fluorescing) SFP. In some embodiments, the SFP detector is a SFP 1-9 detector comprising or consisting of a consensus amino acid sequence set forth as: X₁X₂X₃GEELFTGVVPILIELDGDVNGHKFFVRGEGEGDATX₄GKLSLKFICTTGKLPVPWPTLVTTLT YGVQCFSRYPDHMKRHDFFKSAMPEGYVQERTIX₅FKDDGTYKTRAEVKFEGDTLVNRIELKGIDF KEDGNILGHKLEYNFNX₆HX₇VYITADKQX₈NGIKANFTIRHNVEDGSVQLAX₉HYQQNTPIGDGPVX₁₀X₁₁X₁₂, wherein X₁ is M or no amino acid; X₂ is R or no amino acid; X₃ is K or no amino acid; X₄ is I or N; X₅ is Y or S; X₆ is P or S; X₇ is N or K; X₈ is N or K; X₉ is E or D, X₁₀ is L or no amino acid; X₁₁ is L or no amino acid; and X₁₂ is P or no amino acid (SEQ ID NO: 1), wherein the polypeptide complements with a SFP10 tag and a SFP 11 tag to form a fluorescent SFP complex.

In further embodiments, the SFP 1-9 detector comprises or consists of the amino acid sequence set forth as:

MRKGEELFTGVVPILIELDGDVNGHKFFVRGEGEGDATNGKLSLKFICTTGKLPVPWPTLVTTLTYG VQCFSRYPDHMKRHDFFKSAMPEGYVQERTISFKDDGTYKTRAEVKFEGDTLVNRIELKGIDFKED GNILGHKLEYNFNSHNVYITADKQKNGIKANFTIRHNVEDGSVQLADHYQQNTPIGDGPVLLP (SEQ ID NO: 2; GFP1-9 OPT WT), wherein the polypeptide complements with a SFP10 tag and a SFP 11 tag to form a fluorescent SFP complex.

In more embodiments, the SFP 1-9 detector comprises or consists of the amino acid sequence set forth as:

MRKGEELFTGVVPILIELDGDVNGHKFFVRGEGEGDATIGKLSLKFICTTGKLPVPWPTLVTTLTYG VQCFSRYPDHMKRHDFFKSAMPEGYVQERTIYFKDDGTYKTRAEVKFEGDTLVNRIELKGIDFKED GNILGHKLEYNFNSHKVYITADKQNNGIKANFTIRHNVEDGSVQLADHYQQNTPIGDGPVLLP (SEQ ID NO: 3; GFP1-9 OPT1), wherein the polypeptide complements with a SFP10 tag and a SFP 11 tag to form a fluorescent SFP complex.

In additional embodiments, the SFP 1-9 detector comprises or consists of the amino acid sequence set forth as:

MRKGEELFTGVVPILIELDGDVNGHKFFVRGEGEGDATIGKLSLKFICTTGKLPVPWPTLVTTLTYG VQCFSRYPDHMKRHDFFKSAMPEGYVQERTIYFKDDGTYKTRAEVKFEGDTLVNRIELKGIDFKED GNILGHKLEYNFNPHNVYITADKQKNGIKANFTIRHNVEDGSVQLAEHYQQNTPIGDGPVLLP (SEQ ID NO: 4; GFP1-9 OPT2), wherein the polypeptide complements with a SFP10 tag and a SFP 11 tag to form a fluorescent SFP complex.

Also provided are polypeptides comprising a SFP 1-9 detector comprising an amino acid sequence having at least 95% (such as at least 96%, at least 97%, at least 98%, or at least 99%) sequence identity with a polypeptide comprising the amino acid sequence set forth as SEQ ID NO: 2, SEQ ID NO: 3, or SEQ ID NO: 4, wherein the polypeptide complements with a SFP 10 tag and a SFP 11 tag to form a fluorescent SFP complex.

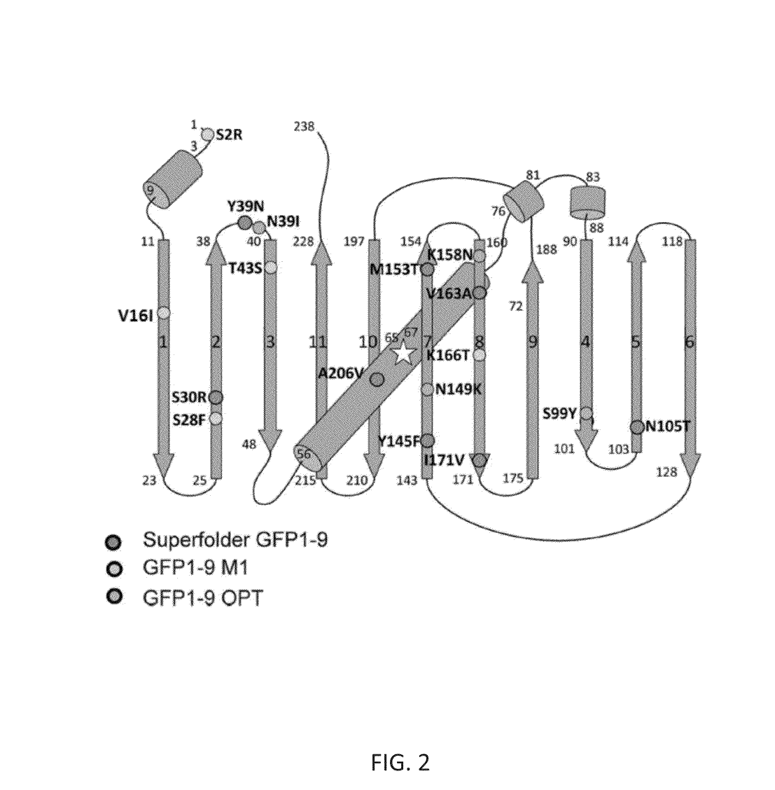

The three-dimensional structure of GFP is well known in the art (see, e.g., Ormo et al., Science, 273:1392-5, 1996). Based on the known three dimensional structure of GFP, the person of ordinary skill in the art can design variants of the disclosed GFP1-9 detectors that would not affect the function of the detector. For example, amino acid substitutions (such as conservative amino acid substitutions) to the connecting loops between the beta strands of the eleven stranded beta-barrel GFP structure are possible. In some embodiments, amino acid substitutions (such as conservative amino acid substitutions) to the connecting loops between the beta strands of the eleven stranded beta-barrel GFP structure are possible. In some embodiments, the polypeptide comprising a SFP 1-9 detector comprises an amino acid sequence having at least 95% (such as at least 96%, at least 97%, at least 98%, or at least 99%) sequence identity with a polypeptide comprising the amino acid sequence set forth as SEQ ID NO: 2, SEQ ID NO: 3, or SEQ ID NO: 4, wherein the polypeptide complements with a SFP 10 tag and a SFP 11 tag to form a fluorescent SFP complex, and wherein the variation specified occurs in the connecting loops between the beta strands of the eleven stranded beta-barrel GFP structure. In some embodiments, the polypeptide comprising a SFP 1-9 detector comprises an amino acid sequence having at least 95% (such as at least 96%, at least 97%, at least 98%, or at least 99%) sequence identity with a polypeptide comprising the amino acid sequence set forth as SEQ ID NO: 2, SEQ ID NO: 3, or SEQ ID NO: 4, wherein the polypeptide complements with a SFP 10 tag and a SFP 11 tag to form a fluorescent SFP complex, and wherein the variation specified does not occur in any one of the residues listed in FIG. 2.

A polypeptide comprising a SFP detector can vary in length according to the specific application. For example, in some embodiments, a polypeptide including a SFP detector includes a minimum length, such as at least 200 (such as at least 250, at least 300, at least 350, at least 400, at least 450, at least 500, at least 550, at least 600, at least 650, at least 700, at least 750, at least 800, at least 850, at least 900, at least 950, or at least 1000) amino acids in length, wherein the polypeptide comprises a SFP detector as described herein and wherein the SFP detector retains the ability to complement with a SFP tag to form a functional fluorescent protein. In further embodiments, a polypeptide comprising a SFP detector includes a maximum length, such as no more than 200 (such as no more than 250, no more than 300, no more than 350, no more than 400, no more than 450, no more than 500, no more than 550, no more than 600, no more than 650, no more than 700, no more than 750, no more than 800, no more than 850, no more than 900 no more than 950, or no more than 1000) amino acids in length, wherein the polypeptide comprises a SFP detector as described herein and wherein the SFP detector retains the ability to complement with a SFP tag to form a functional fluorescent protein.

In some examples, the polypeptides comprising SFP detectors may be fused to a subcellular localization element, for example as described in U.S. App. Pub. Nos. 2005/0221343 and 2012/0282643; PCT Pub. No. WO/2005/074436, and U.S. Pat. Nos. 7,666,606; and 7,585,636, each of which is incorporated herein in its entirety). The skilled artisan is familiar with methods of generating a polypeptide comprising a SFP detector fused to a subcellular localization element. In some examples, the subcellular localization element is fused to the N-terminus, the C-terminus or an internal portion of the SFP detector.

In some examples, the SFP detector is fused to another protein of interest.

As disclosed in Example 1, the disclosed GFP1-9 detectors exhibit minimal self-assembly with the GFP10 tag and the GFP11 tag, unless these tags are brought into close proximity with one another, for example by linkage to the N- and C-terminus of the same protein, or by linkage to interacting protein pairs. Therefore, in some embodiments, incubation of an equal molar ratio of GFP1-9 detector (e.g., comprising the amino acid sequence set forth as SEQ ID NO: 1, SEQ ID NO: 2, SEQ ID NO: 3, or SEQ ID NO: 4), a GFP10 tag (e.g., comprising the amino acid sequence set forth as SEQ ID NO: 11, SEQ ID NO: 12, or SEQ ID NO: 13), and a GFP11 tag (e.g., comprising the amino acid sequence set forth as SEQ ID NO: 14 or SEQ ID NO: 15) under suitable conditions (e.g., as described in Example 1, and with 200 nM GFP10 tag, 200 nM GFP 11 tag, and <800 μm GFP 1-9 detector) results in a level of fluorescence of no more than 25%, such as no more than 20%, no more than 15%, no more than 10%, or no more than 5% of the fluorescence compared to a control level of fluorescence. The control level of fluorescent can be the level of fluorescence from incubation of an equal molar ratio of corresponding GFP1-9 detector, with corresponding GFP10 tag and GFP11 tag fused to the N- and C-terminus of protein, for example as described in Example 1. Suitable conditions for complementing GFP detectors and GFP tags are known to the person of ordinary skill in the art (see, e.g., U.S. App. Pub. Nos. 2005/0221343 and 2012/0282643; PCT Pub. No. WO/2005/074436, and U.S. Pat. Nos. 7,666,606; and 7,585,636), and further described herein (see the Examples).

SFP tags for use with the disclosed SFP detectors are provided and are known in the art (see, e.g., U.S. App. Pub. Nos. 2005/0221343 and 2012/0282643; PCT Pub. No. WO/2005/074436, and U.S. Pat. Nos. 7,666,606; and 7,585,636, each of which is incorporated by reference herein in its entirety).

In some embodiments, a SFP 1-9 detector (such as a polypeptide comprising the amino acid sequence set forth as any one of SEQ ID NOs: 1-4) is complemented with a SFP 10 tag and an SFP 11 tag selected from Table 1.

TABLE 1
Exemplary SFP tags

| SFP tag | Sequence | SEQ ID NO |
|---|---|---|
| GFP10 SF | MGLPDNHYLSTQSVLSKDPN | 11 |
| GFP10 M1 | MDLPDNHYLSTQTILLKDLN | 12 |
| GFP10 M2 | MDLPDDHYLSTQTILSKDLN | 13 |
| GFP11SF | EKRDHMVLLEFVTAAGITGAS | 14 |
| GFP11M4 | EKRDHMVLLEYVTAAGITDAS | 15 |

It will be understood that the SFP tag can be joined to an unrelated polypeptide, for example in methods of detecting protein localization or detecting protein-protein interaction. The SFP tag can be joined to a polypeptide of interest by means known to the person of ordinary skill in the art, for example, by joining the SFP tag and the polypeptide of interest by a peptide linker. Suitable peptide linkers are known to the person of ordinary skill in the art and further disclosed herein

Typically, when fused to a polypeptide (e.g., a test protein), a SFP tag is substantially non-perturbing to the structure of the test protein. SFP tags can be engineered to be less perturbing to fusion protein folding and solubility relative to the same proteins fused to the full-length fluorescent protein (see, e.g., Cabantous et al., Nat. Biotechnol., 23:102-107, 2005; Pedelacq et al., Nat. Biotechnol., 24:79-88, 2006).

In some examples, the SFP tag and SFP detector are based on a circular permutant of GFP, for example as described herein. Since GFP and GFP-like proteins share a conserved 11 β-strand barrel structure, 11 possible circular permutant topologies are possible, wherein new N- and C-termini are introduced within the turns between secondary structure elements and the native N- and C-termini are joined, typically via a short linker polypeptide. The generation of circular permutants of GFP variants may be accomplished using primer-based PCR (and similar methodologies), for example as described in the U.S. patent application Ser. No. 10/973,693, which is incorporated by reference herein in its entirety.

In several embodiments, the SFP detectors disclosed herein include nine contiguous β-strands of GFP, with one or more amino acid substitutions that provide optimizing properties. Thus, additional SFP detectors based on circular permutants of GFP that include nine contiguous β-strands of GFP and include the one or more substitutions located on those β-strands can also be generated. The detectors can be complemented with the remaining two β-strands of GFP (the SFP tags) to form a fluorescent protein. In some embodiments, the SFP detector includes nine contiguous β-strands of a circular permutant GFP including the GFP1-9 WT, GFP1-9 OPT1, or GFP1-9 OPT2 mutations disclosed herein. For example, the SFP detector can comprise or consist of an amino acid sequence set forth as SEQ ID NO: 65 (GFP2-10 OPT1), SEQ ID NO: 66 (GFP3-11 OPT1), SEQ ID NO: 67 (GFP4-1a OPT1), SEQ ID NO: 68 (GFP4-1b OPT1), SEQ ID NO: 69 (GFP5-2 OPT1), SEQ ID NO: 70 (GFP6-3 OPT1), SEQ ID NO: 71 (GFP7-4a OPT1), SEQ ID NO: 72 (GFP7-4b OPT1), SEQ ID NO: 73 (GFP7-4c OPT1), SEQ ID NO: 74 (GFP8-5 OPT1), SEQ ID NO: 75 (GFP9-6 OPT1), SEQ ID NO: 76 (GFP10-7a OPT1), SEQ ID NO: 77 (GFP10-7b OPT1), or SEQ ID NO: 78 (GFP11-8 OPT1).

In additional embodiments, the SFP detector can comprise or consist of an amino acid sequence at least 80% (e.g., at least 85%, at least 90%, or at least 95%) identical to SEQ ID NO: 65 (GFP2-10 OPT1), SEQ ID NO: 66 (GFP3-11 OPT1), SEQ ID NO: 67 (GFP4-1a OPT1), SEQ ID NO: 68 (GFP4-1b OPT1), SEQ ID NO: 69 (GFP5-2 OPT1), SEQ ID NO: 70 (GFP6-3 OPT1), SEQ ID NO: 71 (GFP7-4a OPT1), SEQ ID NO: 72 (GFP7-4b OPT1), SEQ ID NO: 73 (GFP7-4c OPT1), SEQ ID NO: 74 (GFP8-5 OPT1), SEQ ID NO: 75 (GFP9-6 OPT1), SEQ ID NO: 76 (GFP10-7a OPT1), SEQ ID NO: 77 (GFP10-7b OPT1), or SEQ ID NO: 78 (GFP11-8 OPT1). The remaining two strands of the circular permutant can be used as SFP tags to complement with the SFP detector to generate a fluorescent protein. For more information concerning SFP detectors and tags based on circular permutants of GFP, see U.S. Pat. App. Pub. No. 2005/0221343 and PCT Pub. No. WO/2005/074436, each of which is incorporated by reference herein.

In some examples, the polypeptides and the nucleic acid molecules disclosed herein are isolated polypeptides or isolated nucleic acid molecules.

C. Superfolder Cherry Fluorescent Protein (sfCherry)

Also disclosed herein are polypeptides comprising novel fluorescent proteins. In some embodiments the polypeptide includes a sfCherry fluorescent protein, which is a 11-stranded β-barrel fluorescent protein with substantially the same excitation and emission wavelengths compared to the Cherry fluorescent protein (see, e.g., Example 2 and Shaner et al., Nat. Biotechnol., 22, 1567-1572, 2004), but with improved folding characteristics and increased solubility.

In some embodiments, a polypeptide is provided that a sfCherry fluorescent protein comprising the amino acid sequence set forth as:

EEDNMAIIKEFMRFKVHMEGSVNGHEFEIEGEGEGHPYEGTQTAKLKVTKGGPLPFAWDILSPQFM YGSKAYVKHPADIPDYLKLSFPEGFTWERVMNFEDGGVVTVTQDSSLQDGEFIYKVKLLGTNFPSD GPVMQKKTMGWEASTERMYPEDGALKGEINQRLKLKDGGHYDAEVKTTYKAKKPVQLPGAYNV DIKLDITSHNEDYTIVEQYERAEGRHSTGG (SEQ ID NO: 8). In some embodiments the polypeptide comprising the sfCherry fluorescent protein fluoresces with an excitation wavelength range of 530-585 nm and a emission wavelength of 610-670 nm.

Also provided are polypeptides comprising a an amino acid sequence having at least 95% (such as at least 96%, at least 97%, at least 98%, or at least 99%) sequence identity with a polypeptide comprising the amino acid sequence set forth as SEQ ID NO: 8, wherein the polypeptide is a fluorescent protein. The three-dimensional structure of GFP is well known in the art (see, e.g., Ormo et al., Science, 273:1392-5, 1996). Based on the known three dimensional structure of GFP, the person of ordinary skill in the art can design variants of the disclosed sfCherry that would not affect the function of the fluorescent protein. For example, amino acid substitutions (such as conservative amino acid substitutions) to the connecting loops between the beta strands of the eleven stranded beta-barrel GFP structure are possible. In some embodiments, amino acid substitutions (such as conservative amino acid substitutions) to the connecting loops between the beta strands of the eleven stranded beta-barrel GFP structure are possible. In some embodiments, the polypeptide comprising a sfCherry fluorescent protein comprises an amino acid sequence having at least 95% (such as at least 96%, at least 97%, at least 98%, or at least 99%) sequence identity with a polypeptide comprising the amino acid sequence set forth as SEQ ID NO: 8, wherein the polypeptide wherein the polypeptide is a fluorescent protein, and wherein the variation specified occurs in the connecting loops between the beta strands of the eleven stranded beta-barrel GFP structure.

A polypeptide comprising a sfCherry protein can vary in length according to the specific application. For example, in some embodiments, a polypeptide including a sfCherry protein includes a minimum length, such as at least 225 (such as at least 250, at least 300, at least 350, at least 400, at least 450, at least 500, at least 550, at least 600, at least 650, at least 700, at least 750, at least 800, at least 850, at least 900, at least 950, or at least 1000) amino acids in length, wherein the polypeptide is a fluorescent protein. In further embodiments, a polypeptide comprising a sfCherry protein includes a maximum length, such as no more than 225 (such as no more than 250, no more than 300, no more than 350, no more than 400, no more than 450, no more than 500, no more than 550, no more than 600, no more than 650, no more than 700, no more than 750, no more than 800, no more than 850, no more than 900 no more than 950, or no more than 1000) amino acids in length, wherein the polypeptide is a fluorescent protein.

In some examples, the polypeptides comprising a sfCherry protein may be fused to a subcellular localization element, for example as described in U.S. App. Pub. Nos. 2005/0221343 and 2012/0282643; PCT Pub. No. WO/2005/074436, and U.S. Pat. Nos. 7,666,606; and 7,585,636, each of which is incorporated herein in its entirety). The skilled artisan is familiar with methods of generating a polypeptide comprising a sfCherry protein fused to a subcellular localization element. In some examples, the subcellular localization element is fused to the N-terminus, the C-terminus or an internal portion of the sfCherry protein.

In some examples, the sfCherry protein is fused to another protein of interest.

### D. Protease Sensors

Also provided is a sensor for protease activity that is based on the SFP detector/tag embodiments disclosed herein. In some embodiments, a circular permutant of GFP including, from N- to C-terminus, β-strand 10, linker, β-strand 11, linker, β-strands 1-9, linker, protease cleavage site, linker, and a second copy of β-strand 10 including a T203Y substitution is provided. Expression of this construct leads to a predominantly yellow fluorescing protein. Cleavage at the protease site separates the C-terminal yellow β-strand including the T203Y substitution from the remaining portion of the construct. The construct remains intact until illuminated by violet light, whereupon the C-terminal strand dissociates, and is replaced by the N-terminal green version of strand 10, resulting in conversion of yellow to green, thereby detecting the presence of the protease activity. In several embodiments the protease site is a caspase-3 protease site, for example, including the amino acid sequence set forth as DEVDG (SEQ ID NO: 82). In some examples the protease sensor is a caspase-3 protease sensor and includes the amino acid sequence set forth as SEQ ID NO: 80. An exemplary nucleotide sequence encoding SEQ ID NO: 80 is provided as SEQ ID NO: 79.

### E. Methods

Methods of using the disclosed SFPs and fluorescent proteins are also provided. For example, the disclosed SFP detectors are useful in methods of detecting protein-protein interactions between a first test polypeptide and a second test polypeptide. For example, as disclosed in Example 1, the disclosed GFP1-9 detectors exhibit minimal self-assembly with the GFP10 tag and the GFP11 tag, unless these tags are brought into relatively close proximity with one another, for example by linkage to the N- and C-terminus of the same protein, or by linkage to interacting protein pairs.

In some embodiments, a method of detecting a protein-protein interaction between a first test polypeptide and a second test polypeptide is provided. The method comprises providing a SFP detector as disclosed herein, for example, comprising at least nine contiguous β-strands of a recombinant green fluorescent protein comprising the consensus amino acid sequence set forth as SEQ ID NO: 1 or a circular permutant thereof, wherein the SFP detector can complement with the remaining two β-strands of the SFP to form a fluorescent protein. A first test polypeptide fused to a first SFP tag comprising the first of the two remaining β-strands; and a second test polypeptide fused to a second SFP tag comprising the second of the two remaining β-strands are also provided. If the first test polypeptide binds to the second polypeptide, then the SFP detector, the first SFP tag, and the second SFP tag will complement to form a fluorescent protein complex. Complementation of these SFP fragments is facilitated by the binding of the first test polypeptide to the second test polypeptide, which brings the SFP tags within close proximity to one another. The proximity of the two tags enhances complementation with the SFP detector. The provided proteins are assayed for fluorescence, and detection of fluorescence detects the protein-protein interaction.

In some embodiments, the SFP detector can comprise or consist of the consensus amino acid sequence set forth as SEQ ID NO: 1. In some embodiments, the SFP detector comprises or consists of the amino acid sequence set forth as SEQ ID NO: 2 (GFP1-9 OPT WT), SEQ ID NO: 3 (GFP1-9 OPT1), SEQ ID NO: 4 (GFP1-9 OPT2), SEQ ID NO: 65 (GFP2-10 OPT1), SEQ ID NO: 66 (GFP3-11 OPT1), SEQ ID NO: 67 (GFP4-1a OPT1), SEQ ID NO: 68 (GFP4-1b OPT1), SEQ ID NO: 69 (GFP5-2 OPT1), SEQ ID NO: 70 (GFP6-3 OPT1), SEQ ID NO: 71 (GFP7-4a OPT1), SEQ ID NO: 72 (GFP7-4b OPT1), SEQ ID NO: 73 (GFP7-4c OPT1), SEQ ID NO: 74 (GFP8-5 OPT1), SEQ ID NO: 75 (GFP9-6 OPT1), SEQ ID NO: 79 (GFP10-7a OPT1), SEQ ID NO: 77 (GFP10-7b OPT1), or SEQ ID NO: 78 (GFP11-8 OPT1).

In some embodiments, the first polypeptide can be fused to a GFP10 tag and the second polypeptide can be fused to a GFP11 tag.

In an additional embodiment, the first and second test proteins do not interact directly with each other, but instead form a tertiary complex with a third, untagged protein. In this embodiment, complementation of the SFP fragments is facilitated by the binding of the first test polypeptide and the second test polypeptide to the third protein, which brings the SFP tags with close proximity to one another. The proximity of these two tags enhances complementation with the SPF detector. In this embodiment, detecting the fluorescence of the complemented split-GFP protein complex detects the protein interaction between the first, second, and third polypeptide.

In some embodiments, a method is provided for detecting a conformational change in the structure of a test protein. In such embodiments, the two SFP tags (e.g., GFP10 tag and GFP11 tag), are fused to either end (such as the N- and C-terminus, respectively) of a test protein. If the test protein adopts a structural conformation that places the GFP10 and GFP11 tags within close proximity to each other, then the proximity of these two tags enhances complementation with the SFP (e.g., GFP1-9) detector. If the test protein does not adopt a structural conformation that places the SFP tags within close proximity to each other, then the two tags will not complement with the SFP detector. Detecting a change in the level of fluorescence depending on varied conditions (buffer conditions, or amino acid changes to the test protein) detects a conformational change in the structure of the test protein. It will be appreciated that such methods can also be used to determine solubility of a test protein.

It will be understood that some background fluorescence can be present. For example, detecting the fluorescence (or lack thereof) of the complemented split-GFP protein can include detecting an increase (or decrease) in fluorescence compared to a control, such as a control described in Example 1. Additional description of methods of using the disclosed GFP1-9 detectors in methods of detecting protein-protein interactions, as well as methods of detecting conformational changes in a single protein, is provided in Example 1, below.

Additionally, the person of skill in the art will understand that the disclosed SFPs and fluorescent proteins have utility, for example, in known methods of using SFPs and fluorescent proteins. Such methods are known to the person of ordinary skill in the art (see, e.g., in U.S. App. Pub. Nos. 2005/0221343 and 2012/0282643; PCT Pub. No. WO/2005/074436, and U.S. Pat. Nos. 7,666,606; and 7,585,636, each of which is incorporated herein in its entirety), and are further described herein (see Example 1 and Example 2).

F. Nucleic acid molecules, DNA Constructs, Expression Vectors and Host Cells

Additionally disclosed are exemplary nucleic acid molecules encoding the SFP and Fluorescent proteins described herein.

In more embodiments, a nucleic acid molecule encoding a SFP detector including at least nine contiguous β-strands of a recombinant green fluorescent protein comprising the consensus amino acid sequence set forth as SEQ ID NO: 1 or a circular permutant thereof is provided. In some embodiments, a nucleic acid molecule encoding a disclosed GFP1-9 detector is provided. For example, the nucleic acid molecule can include a nucleotide sequence set forth as any one of SEQ ID NO: 5, SEQ ID NO: 6, or SEQ ID NO: 7 (listed below), which encode the amino acid sequence set forth as SEQ ID NO: 2, SEQ ID NO: 3, or SEQ ID NO: 4, respectively.

GFP1-9 OPT WT (SEQ ID NO: 5):
ATGAGGAAAGGAGAAGAACTTTTCACTGGAGTTGTCCCAATTCTTATTGAATTAGATGGTGATG
TTAATGGGCACAAATTTTTTGTCCGTGGAGAGGGTGAAGGTGATGCTACAAACGGAAAACTCA
GCCTTAAATTTATTTGCACTACTGGAAAACTACCTGTTCCATGGCCAACACTTGTCACTACTCTG
ACCTATGGTGTTCAATGCTTTTCCCGTTATCCGGATCACATGAAACGGCATGACTTTTTCAAGAG
TGCCATGCCCGAAGGTTATGTACAGGAACGCACTATATCTTTCAAAGATGACGGGACCTACAAG
ACGCGTGCTGAAGTCAAGTTTGAAGGTGATACCCTTGTTAATCGTATCGAGTTAAAGGGTATTG
ATTTTAAAGAAGATGGAAACATTCTTGGACACAAACTCGAGTACAACTTTAACTCACACAATGT
ATACATCACGGCAGACAAACAAAAGAATGGAATCAAAGCTAACTTCACAATTCGCCACAACGT
TGAAGATGGTTCCGTTCAACTAGCAGACCATTATCAACAAAATACTCCAATTGGCGATGGCCCT
GTCCTTTTACCA
GFP1-9 OPT1 (SEQ ID NO: 6):
ATGAGGAAAGGAGAAGAACTTTTCACTGGAGTCGTCCCAATTCTTATTGAATTAGATGGTGATG
TTAATGGGCACAAATTTTTTGTCCGTGGAGAGGGTGAAGGTGATGCTACAATCGGAAAACTCAG
CCTTAAATTTATTTGCACTACTGGAAAACTACCTGTTCCATGGCCAACACTTGTCACTACTCTGA
CCTATGGTGTTCAATGCTTTTCCCGTTATCCGGATCACATGAAACGGCATGACTTTTTCAAGAGT
GCCATGCCCGAAGGTTATGTACAGGAACGCACTATATATTTCAAAGATGACGGGACCTACAAG
ACGCGTGCTGAAGTCAAGTTTGAAGGTGATACCCTTGTTAATCGTATCGAGTTAAAGGGTATTG
ATTTTAAAGAAGATGGAAACATTCTTGGACACAAACTCGAGTACAACTTTAACTCACACAAAGT
ATACATCACGGCAGACAAACAAAATAATGGAATCAAAGCTAACTTCACAATTCGCCACAACGT
TGAAGATGGTTCCGTTCAACTAGCAGACCATTATCAACAAAATACTCCAATTGGCGATGGCCCT
GTCCTTTTACCA
GFP1-9 OPT2 (SEQ ID NO: 7):
ATGAGGAAAGGAGAAGAACTTTTCACTGGAGTTGTCCCAATTCTTATTGAATTAGATGGTGATG
TTAATGGGCACAAATTTTTTGTCCGTGGAGAGGGTGAAGGTGATGCTACAATCGGAAAACTCAG
CCTTAAATTTATTTGCACTACTGGAAAACTACCTGTTCCATGGCCAACACTTGTCACTACTCTGA
CCTATGGTGTTCAATGCTTTTCCCGTTATCCGGATCACATGAAACGGCATGACTTTTTCAAGAGT
GCCATGCCCGAAGGTTATGTACAGGAACGCACTATATATTTCAAAGATGACGGGACCTACAAG
ACGCGTGCTGAAGTCAAGTTTGAAGGTGATACCCTTGTTAATCGTATCGAGTTAAAGGGTATTG
ATTTTAAAGAAGATGGAAACATTCTTGGACACAAACTCGAGTACAACTTTAACCCACACAATGT
ATACATCACGGCAGACAAACAAAAGAATGGAATCAAAGCTAACTTCACAATTCGCCACAATGT
TGAAGATGGTTCCGTTCAACTAGCAGAGCATTATCAACAAAATACTCCAATCGGCGATGGCCCT
GTCCTTTTACCA

In more embodiments, a nucleic acid molecule can include a nucleotide sequence set forth as any one of SEQ ID NOs: 51-64 (listed in Example 3), which encode the SFP detectors provided as SEQ ID NOs: 65-78, respectively.

Additionally disclosed are exemplary nucleic acid molecules encoding the sfCherry fluorescent protein. In some embodiments, the nucleic acid molecule includes a nucleotide sequence set forth as SEQ ID NO: 9 or SEQ ID NO: 10, which encode the amino acid sequence set forth as SEQ ID NO: 8. SEQ ID NO: 10 (sfCherry recoded) is a nucleic acid sequence that has been codon altered compared to SEQ ID NO: 9 and encodes the same polypeptide sequence as SEQ ID NO: 9.

(encoding sfCherry):
SEQ ID NO: 9
GAAGAAGATAATATGGCAATTATCAAGGAGTTCATGCGCTTCAAGGTGCACATGGAGGGCTCC
GTGAACGGCCACGAGTTCGAGATCGAGGGCGAGGGCGAGGGCCACCCCTACGAGGGCACCCA
GACCGCCAAGCTGAAGGTGACCAAGGGCGGCCCCCTGCCCTTCGCCTGGGACATCCTGTCCCCT
CAGTTCATGTACGGCTCCAAGGCCTACGTGAAGCACCCCGCCGACATCCCCGACTACTTGAAGC
TGTCCTTCCCCGAGGGCTTCACGTGGGAGCGCGTGATGAACTTCGAGGACGGCGGCGTGGTGAC
CGTGACCCAGGACTCCTCCCTGCAGGACGGCGAGTTCATCTACAAGGTGAAGCTGCTCGGCACC
AACTTCCCCTCCGACGGCCCCGTAATGCAGAAGAAGACCATGGGCTGGGAGGCCTCCACTGAG
CGGATGTACCCCGAGGACGGCGCCCTGAAGGGCGAGATCAATCAGAGGCTGAAGCTGAAGGAC
GGCGGCCACTACGACGCCGAGGTCAAGACCACCTACAAGGCCAAGAAGCCCGTGCAGCTGCCC
GGCGCCTACAATGTGGATATCAAGCTGGACATCACCTCCCACAACGAGGACTACACCATCGTG
GAACAGTACGAGCGCGCCGAGGGCCGCCACTCCACCGGCGGT
(encoding sfCherry):
SEQ ID NO: 10
GAGGAGGATAACATGGCAATTATCAAGGAATTTATGCGATTTAAGGTTCACATGGAGGGTTCTG
TTAATGGGCACGAATTTGAGATCGAAGGAGAGGGTGAAGGTCATCCTTACGAGGGAACACAGA
CCGCTAAATTGAAAGTCACTAAAGGAGGACCTCTTCCATTCGCCTGGGATATACTTTCCCCTCA
GTTTATGTATGGTTCTAAAGCCTATGTCAAACATCCGGCTGACATCCCAGACTATTTGAAGTTGT
CCTTCCCCGAAGGTTTTACATGGGAACGCGTTATGAACTTCGAAGATGGCGGGGTCGTCACGGT
GACACAGGACTCCAGCTTGCAAGATGGTGAGTTTATTTATAAAGTCAAGTTATTAGGTACTAAT
TTTCCATCAGATGGACCCGTTATGCAGAAAAAGACGATGGGCTGGGAGGCATCCACTGAACGC
ATGTACCCAGAAGACGGTGCACTCAAAGGTGAGATCAATCAACGCCTCAAGCTTAAAGATGGT
GGCCATTACGATGCAGAGGTTAAGACAACATATAAGGCAAAAAAGCCTGTCCAGTTACCAGGC
GCCTATAACGTGGACATAAAATTGGACATTACGAGCCATAACGAGGACTACACAATCGTGGAG
CAGTATGAGCGTGCAGAGGGTCGTCACAGTACAGGTGGC

In more embodiments, a nucleic acid molecule is provided that encodes a protease sensor as set forth herein. For example, the nucleic acid molecule can include a sequence set forth as SEQ ID NO: 79, which encodes a protease sensor comprising the amino acid sequence set forth as SEQ ID NO: 80.

Nucleic acid molecules encoding one or more test proteins, fluorescent proteins, SFP detectors, tags, and fusions of two of more thereof can be included in one or more expression vectors to direct expression of the corresponding nucleic acid sequence. Thus, other expression control sequences including appropriate promoters, enhancers, transcription terminators, a start codon at the front of a protein-encoding sequence, splicing signal for introns, maintenance of the correct reading frame of that gene to permit proper translation of mRNA, and stop codons can be included in an expression vector. Generally, expression control sequences include a promoter, a minimal sequence sufficient to direct transcription.

Nucleic acid sequences encoding test proteins, SFP tags, SFP detectors and fusions of two or more thereof, etc., may be included in an expression vector to direct expression of the corresponding nucleic acid sequence. Optionally, the nucleic acid sequences encoding an SFP tag, affinity tag and/or SFP detector may be operably linked to the nucleic acid encoding a test protein, such that expression from the expression vector results in a fusion protein of the test protein fused to the SFP tag, affinity tag and/or SFP detector.

As will be appreciated by the skilled artisan, expression vectors used to express test proteins, SFP tags, affinity tags, SFP detectors and fusions thereof must be compatible with the host cell in which the proteins are to be expressed. Similarly, various promoter systems are available and should be selected for compatibility with cell type, strain, etc. Codon optimization techniques may be employed to adapt sequences for use in other cells, as is well known.

The expression vector typically contains an origin of replication, a promoter, as well as specific genes which allow phenotypic selection of the transformed cells (e.g., an antibiotic resistance cassette). Vectors suitable for use include, but are not limited to, the pMSXND expression vector for expression in mammalian cells (Lee and Nathans, J. Biol. Chem. 263:3521, 1988). Generally, the expression vector will include a promoter. The promoter can be inducible or constitutive. In one embodiment, the promoter is a heterologous promoter.

Unlike constitutive promoters, an inducible promoter is not always active. Some inducible promoters are activated by physical stimuli, such as the heat shock promoter. Others are activated by chemical stimuli, such as IPTG or Tetracycline (Tet), or galactose. Inducible promoters or gene-switches are used to both spatially and temporally regulate gene expression. Thus, for a typical inducible promoter in the absence of the inducer, there would be little or no gene expression while, in the presence of the inducer, expression should be high (i.e., off/on). The skilled artisan is familiar with inducible promoters and will appreciate which inducible promoters may be used in the embodiments described herein.

In some embodiments, multiple inducible promoters are included on an expression vector, each promoter induced by a different inducer. In other embodiments, multiple expression vectors are included in the host cell, each expression vector comprising an inducible promoter, each inducible promoter induced by a different inducer. In this way, expression of multiple proteins in a host cell can be independently under the control of separate inducible promoters. Thus, in some embodiments, host cells are engineered to express one or more complementary fragments of a SFP, one or more of which are fused to one or more test proteins. The fragments may be expressed simultaneously or sequentially.

Systems of two independently controllable promoters have been described and are well known in the art, and are described herein. See, for example, Lutz and Bujard, Nucleic Acids Res., 25:1203-1210, 1997.

In one example, a vector in which the promoter is under the repression of the Laclq protein and the arabinose inducer/repressor may be used for expression of the SFP detector (e.g., pPROLAR vector available from Clontech, Palo Alto, Calif.). Repression is relieved by supplying IPTG and arabinose to the growth media, resulting in the expression of the SFP detector. In this system, the araC repressor is supplied by the genetic background of the host E. coli cell. For the controlled expression of the test protein-SFP tag fusion, a vector in which the test protein-SFP tag fusion is under the repression of the tetracycline repressor protein may be used (e.g., pPROTET vector; Clontech). In this system, repression is relieved by supplying anhydrotetracycline to the growth media, resulting in the expression of the test protein-SFP tag fusion construct. The tetR and Laclq repressor proteins may be supplied on a third vector, or may be incorporated into the fragment-carrying vectors.

In one example, nucleic acid encoding a test protein, SFP tag, SFP detector or fusion of two or more thereof is located downstream of the desired promoter. Optionally, an enhancer element is also included, and can generally be located anywhere on the vector and still have an enhancing effect. However, the amount of increased activity will generally diminish with distance. Expression vectors including a nucleic acid encoding a test protein, SFP tag, SFP detector or fusion of two or more thereof can be used to transform host cells.

The disclosed embodiments may be applied in virtually any host cell type, including without limitation bacterial cells (e.g., E. coli) and mammalian cells (e.g., CHO cells). Hosts can include isolated microbial, yeast, insect and mammalian cells, as well as cells located in the organism. For example, the host cell may be an E. coli cell, such as an E. coli BL21 (DE3) strain cell. Secretion competent yeast and bacterial cells may be used. The skilled artisan is familiar with such cells. Nucleic acid encoding test proteins, affinity tags, SFP tags, SFP detectors and fusion proteins are typically comprised in an expression vector introduced into the host cells. One limitation is that expression of GFP and GFP-like proteins is compromised in highly acidic environments (i.e., pH=4.0 or less). Likewise, complementation rates are generally inefficient under conditions of pH of 6.5 or lower (see, e.g., U.S. patent application Ser. No. 10/973,693).

A transfected cell is a cell into which (or into an ancestor of which) has been introduced, by means of recombinant DNA techniques, a DNA molecule encoding a protein of interest. Transfection of a host cell with recombinant DNA may be carried out by conventional techniques as are well known in the art. Where the host is prokaryotic, such as E. coli, competent cells which are capable of DNA uptake can be prepared from cells harvested after exponential growth phase and subsequently treated by the CaCl₂ method using procedures well known in the art. Alternatively, MgC12 or RbC1 can be used. Transformation can also be performed after forming a protoplast of the host cell if desired, or by electroporation.

When the host is a eukaryote, such as a CHO cell, such methods of transfection of DNA as calcium phosphate coprecipitates, conventional mechanical procedures such as microinjection, electroporation, insertion of a plasmid encased in a liposome, or virus vectors may be used. Eukaryotic cells can also be cotransformed with DNA sequences encoding the test protein, and a second foreign DNA molecule encoding a selectable phenotype, such as neomycin resistance. Another method is to use a eukaryotic viral vector, such as simian virus 40 (SV40) or bovine papilloma virus, to transiently infect or transform eukaryotic cells and express the protein (see for example, Eukaryotic Viral Vectors, Cold Spring Harbor Laboratory, Gluzman ed., 1982). Other specific, non-limiting examples of viral vectors include adenoviral vectors, lentiviral vectors, retroviral vectors, and pseudorabies vectors.

As will be appreciated by those skilled in the art, the vectors used to express the test proteins, SFP tags, SFP detectors and fusions of two or more thereof disclosed herein must be compatible with the host cell in which the vectors are provided. Similarly, various promoter systems are available and should be selected for compatibility with cell type, strain, etc. Codon optimization techniques may be employed to adapt sequences for use in other cells, as is well known. In some examples, expression of polypeptides may be performed using a cell-free system; such systems are known to the skilled artisan and are commercially available (see, e.g., Cat No. K9901-01, Invitrogen, Corp., Carlsbad, Calif.).

When using mammalian cells for the subcellular localization methods described herein, an alternative to codon optimization is the use of chemical transfection reagents, such as the recently described chariot system (Morris et al., Nature Biotechnol. 19: 1173-1176, 2001). The Chariot™ protein delivery reagent (Activmotif, Corp., Carlsbad, Calif.) may be used to directly transfect a protein into the cytoplasm of a mammalian cell. Thus, this approach would be useful for providing a SFP fragment (e.g., an SFP detector) within a host cell, for instance before, after or during expression of a complementary SFP fragment expressed within the host cell.

### G. Kits

Provided herein are kits useful for the various embodiments described herein. The kits may facilitate the use of SFPs for determining the subcellular localization of a protein, or a protein-protein interaction as described herein. Kits may contain various materials and reagents (e.g., for practicing the methods described herein). For example, a kit may contain reagents including, without limitation, polypeptides or polynucleotides, cell transformation and transfection reagents, reagents and materials for purifying polynucleotides and polypeptides including lysis regents, protein denaturing and refolding reagents, as well as other solutions or buffers useful in carrying out the assays and other methods of the invention. Kits may also include control samples, materials useful in calibrating methods described herein, and containers, tubes, microtiter plates and the like in which assay reactions may be conducted. Kits may be packaged in containers, which may comprise compartments for receiving the contents of the kits, instructions for conducting methods described herein or using the polypeptides and polynucleotides described herein, etc.

For example, a kit may provide one or more SFP fragments or fluorescent proteins as described herein, one or more polynucleotide constructs encoding the one or more SFP fragments or fluorescent proteins, one or more polynucleotide constructs encoding one or more subcellular localization elements as described herein, cell strains suitable for propagating the constructs, cells pre-transformed or stably transfected with constructs encoding one or more SFP fragment or fluorescent proteins s, and reagents for purification of expressed fusion proteins or nucleotide encoding an expressed fusion protein. For example, a kit may provide a nucleic acid construct encoding a SFP tag and a multiple cloning site adjacent thereto, such that an encoding sequence inserted into the multiple cloning site results in a nucleic acid that encodes a protein encoded by the encoding sequence fused with the SFP tag, and instructions for using the nucleic acid (e.g., instructions for carrying out the methods described herein). In another example, a kit may provide a nucleic acid construct encoding a SFP detector as described herein and a multiple cloning site adjacent thereto, such that an encoding sequence inserted into the multiple cloning site results in a nucleic acid molecule that encodes a protein encoded by the encoding sequence fused with the SFP detector and instructions for using the nucleic acid (e.g., instructions for carrying out the methods described herein).

The kit can include a container and a label or package insert on or associated with the container. The label or package insert typically will further include instructions for use of the polypeptide, nucleic acid molecules, or expression vector provided with the kit, for example for use in the methods disclosed herein. The instructional materials may be written, in an electronic form (such as a computer diskette or compact disk) or may be visual (such as video files).

### EXAMPLES

The following examples are provided to illustrate particular features of certain embodiments, but the scope of the claims is not intended to be limited to those features exemplified.

### Example 1

### Tripartite Split-GFP System

This example illustrates a novel tripartite split-GFP system, including a GFP1-9 detector, a GFP10 tag and a GFP 11 tag. As described below, the tripartite split-GFP system can be used for example, to detect protein-protein interactions, and alterations in protein structural conformation.

Monitoring protein-protein interactions in living cells is key to unraveling their roles in numerous cellular processes and various diseases. Previously described split-GFP based sensors suffer from poor folding and/or self-assembly background fluorescence. Here, engineered a micro-tagging system was engineered to monitor protein-protein interactions in vivo and in vitro. The assay is based on tripartite association between two twenty amino-acids long SFP tags, GFP10 and GFP11, fused to interacting protein partners, and the complementary GFP1-9 detector. When the proteins interact, GFP10 and GFP11 self-associate with GFP1-9 to reconstitute a functional GFP. Using coiled-coils and FRB/FKBP12 model systems the sensor was characterized in vitro and in Escherichia coli. The studies are extended to mammalian cells and the FK-506 inhibition of the rapamycin-induced association of FRB/FKBP12 was examined. The small size of these tags and their minimal effect on fusion protein behavior and solubility should enable new experiments for monitoring protein-protein association by fluorescence.

### Introduction

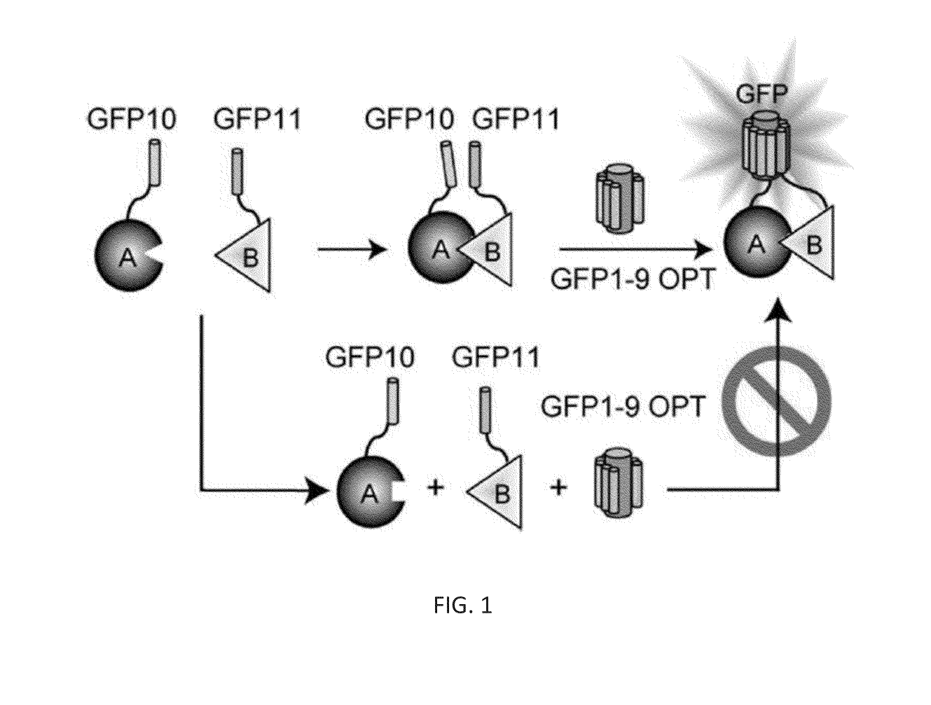

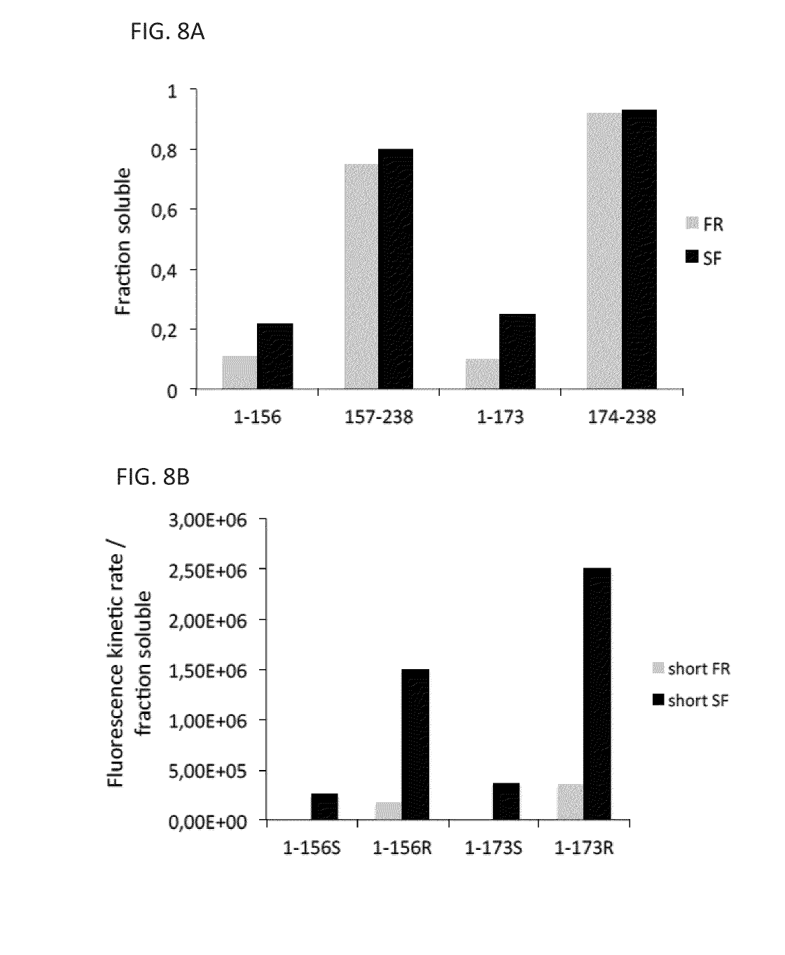

Signal transduction and gene expression often involves protein-protein interactions. Optical tracking of these interactions can help to illuminate regulatory mechanisms and identify aberrant processes in diseases. Fluorescent protein biosensors have been developed to measure protein complexes in living cells (Day, et al., Chem Soc Rev 38, 2887-2921, 2009). For example, bioluminescence resonance energy transfer (FRET and BRET) enable dynamic observations of protein-protein interactions (Pfleger, et al., Nat Methods 3, 165-174, 2006). More recent developments include protein-fragment complementation assays (PCA) than monitor protein-protein interactions by reconstitution of fragments of various enzymes split into two pieces (as fused tags on passenger proteins) including fragments of dihydrofolate reductase (Pelletier, et al., Nat Biotechnol 17, 683-690, 1999), β-galactosidase (Rossi, et al., Proc Natl Acad Sci USA 94, 8405-8410, 1997), β-lactamase (Galarneau, et al., Nat Biotechnol 20, 619-622, 2002), and the firefly and Gaussia luciferases (Shekhawat, et al., Curr Opin Chem Biol 15, 789-797, 2011). Though sensitive, these have the limitation that the observed products diffuse away from the protein interaction site. PCA based on split green fluorescent protein (GFP) and color variants (Hu, et al., Nat Biotechnol 21, 539-545, 2003) is termed bimolecular fluorescence complementation (BiFC). BiFC relies on (i) interactions between bait and prey proteins that bring together two non-fluorescent split protein domains and (ii) subsequent co-folding into the β-barrel structure to form the chromophore (Magliery, et al., J Am Chem Soc 127, 146-157, 2005). Assembly of the GFP fragments is irreversible, the stability of the BiFC enables integration, accumulation, and subsequent detection even of transient interactions and low affinity complexes (Morell, et al., Proteomics 7, 1023-1036, 2007; MacDonald, et al., Nat Chem Biol 2, 329-337, 2006). These remain attached to the interacting proteins, enabling tracking of the complex. However, improving existing BiFCs based on large and bulky fragments is challenging as increasing BiFC fragment solubility and folding can increase background signals from spontaneous assembly of the fluorescent protein fragments (Kodama, et al., Biotechniques 49, 793-805, 2010). For example, BiFC fragments (Hu, et al., Mol Cell 9, 789-798, 2002) obtained by fragmentation of folding-reporter GFP (FR-GFP; Waldo, et al., Nat Biotechnol 17, 691-695, 1999) and superfolder GFP (sfGFP; Pedelacq, et al., Nat Biotechnol 24, 79-88, 2006) at permissive sites 156 and 173 were aggregation-prone and had high backgrounds from self-assembly (FIG. 8). Using a smaller tag can reduce aggregation and folding interference. For example, one split GFP uses a very small 15 amino acid tag for quantification of soluble protein and tracking proteins in vivo (Cabantous, et al., Nat Biotechnol 23, 102-107, 2005; Cabantous, et al., Nat Methods 3, 845-854, 2006; Kaddoum, et al., Biotechniques 49, 727-728, 730, 732, 2010). However the large size of the remaining piece self-assembly of the fragments makes this system less suited for protein-protein interaction studies. A spontaneously assembling split GFP based on GFP10-11 (residues 194-233, the tag) and GFP1-9 (residues 1-193, the detector) was previously developed. Here the engineering of this split GFP to yield an entirely new protein interaction assay based on the association of three fragments of the GFP is described: two short peptides GFP10 (residues 194-212) and GFP11 (residues 213-233) each tagged to one of the interacting partners, and a third large GFP1-9 (residues 1-193) detector fragment (FIG. 1). The characterization of the assay is presented using attractive and repulsive pairs of charged coiled-coils peptides (Tripet, B., et al., Protein Eng 10, 299, 1997) and the rapamycin mediated heterodimerization of FK506 binding protein (FKBP) and FKBP12-rapamycin binding domain (FRB; Banaszynski, et al., J Am Chem Soc 127, 4715-4721, 2005). The assay correctly reports the localization of protein-protein complexes in mammalian cells with very low background fluorescence levels. The chief advantage over BiFC is the small size of the tags (ca. 20 amino acids), and the concomitant reduced interference with passenger protein folding. The tripartite split GFP assay may find broad utility in the visualization of protein complexes especially where bulkier BiFC fragments might impede localization or interfere with folding.

### Results

Engineering a Three-Body Split-GFP System for Improved Solubility and Complementation.

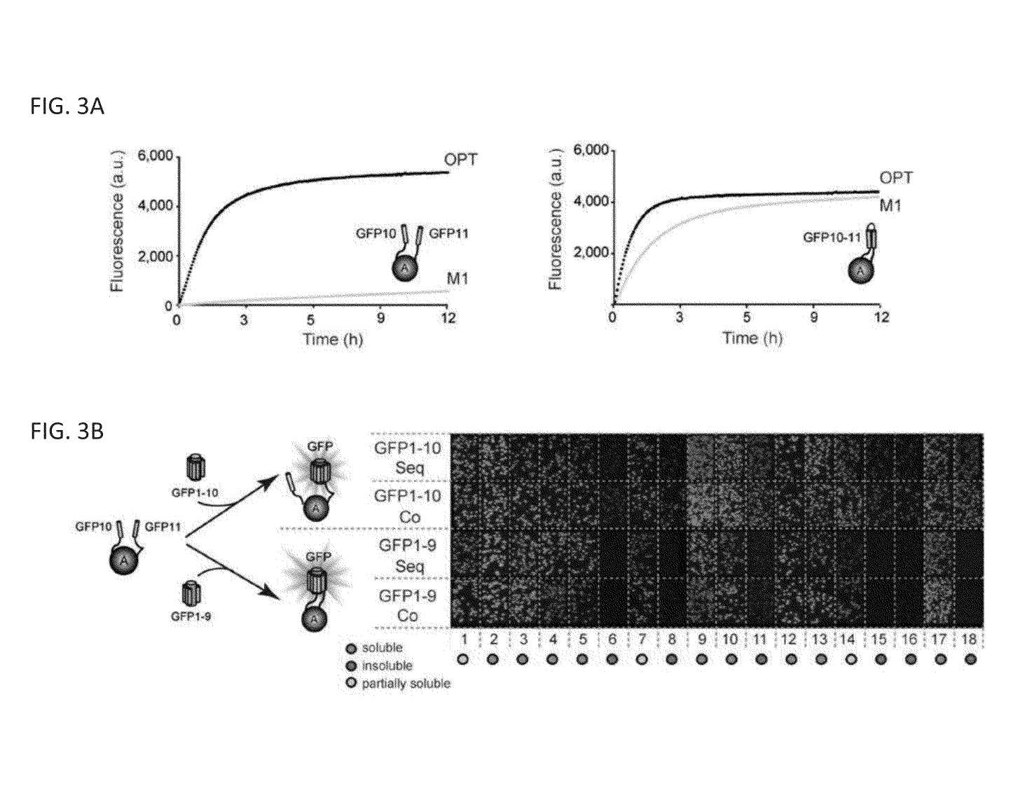

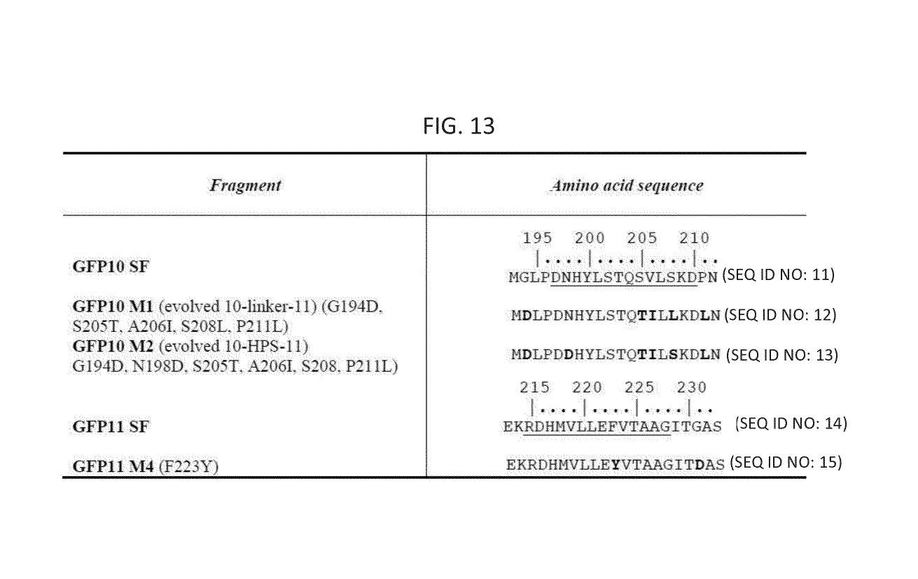

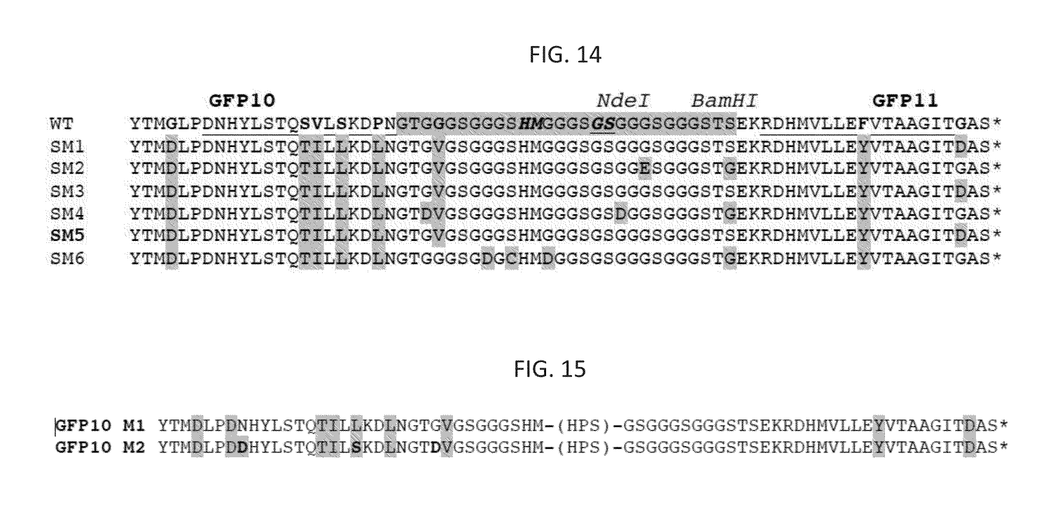

Self-assembling split-GFP fragments corresponding to the C-terminal β-hairpin GFP10-11 of sfGFP(residues 194-238; Pedelacq, et al., Nat Biotechnol 24, 79-88, 2006) and the large fragment GFP1-9 (residues 1-193) were previously identified (FIG. 2). In order to improve GFP1-9 folding efficiency, two rounds of directed evolution were performed and a new variant of GFP1-9 named GFP1-9 M1 (SEQ ID NO: 2; also called GFP1-9 WT) was obtained that contained five additional mutations compared to sfGFP1-9 (FIG. 2, FIG. 14). This bimolecular pair was converted into a three-body split-GFP in a stepwise manner. First, in attempt to destabilize the GFP10-11 β-hairpin self-assembly, a long flexible linker that included a cloning site between GFP10 and GFP11 was inserted and evolved the whole cassette for improved solubility and complementation efficiency with GFP1-9 M1 (See Methods). Next, GFP10 and GFP11 were further spaced out by inserting a partially soluble bait protein, hexylose phosphate synthase or HPS (Cabantous, et al., Nat Biotechnol 23, 102-107, 2005) in the cloning site of the linker HPS alone was ˜60% soluble and was used in the earlier study as a bait protein to improve solubility of split-GFP tags (FIG. 13, FIG. 15; Cabantous, et al., Nat Biotechnol 23, 102-107, 2005). In the final step, GFP1-9 M1 was evolved to recognize this “sandwich” bait topology. When complemented with the optimized GFP10 and GFP11 tags attached to the N and C termini of the HPS (Cabantous, et al., Nat Biotechnol 23, 102-107, 2005), the resulting variant named GFP1-9 OPT1 (SEQ ID NO: 3), exhibited a 40-fold improvement in the rate of formation of fluorescence (FIG. 3A, black line) relative to GFP1-9 (FIG. 3A, gray line). Differences between the GFP1-9 were less obvious when complemented with the GFP10-11 hairpin (FIG. 3A, right panel), presumably due to the reduced entropy of the GFP10-11 hairpin relative to the GFP10-HPS-GFP11 “sandwich” format. GFP1-9 OPT1 amino-acid sequence revealed the presence of four additional mutations relative to GFP1-9 M1 (FIG. 2).

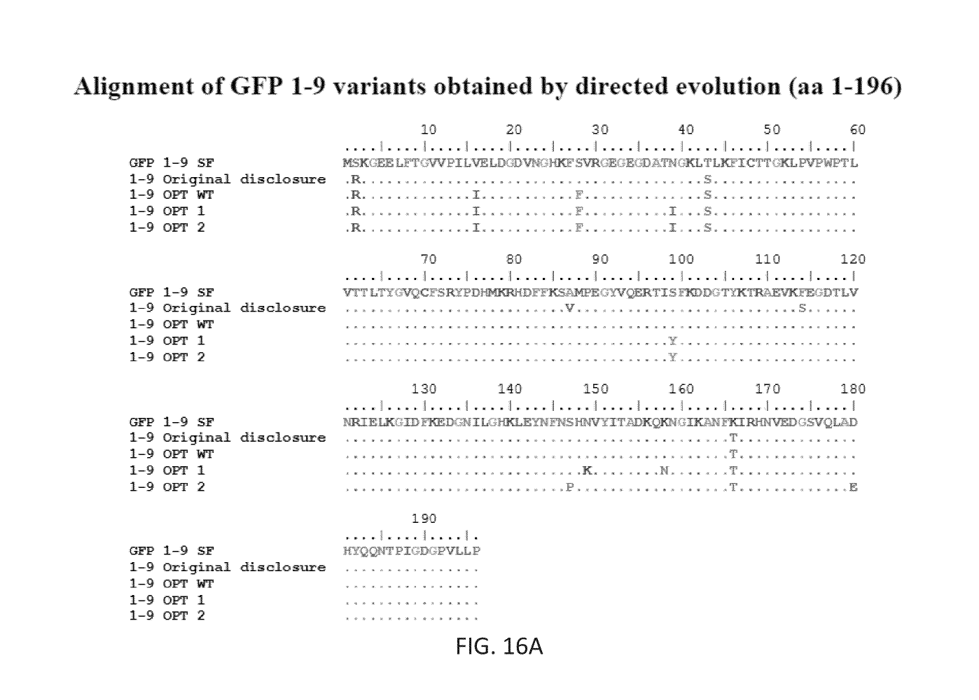

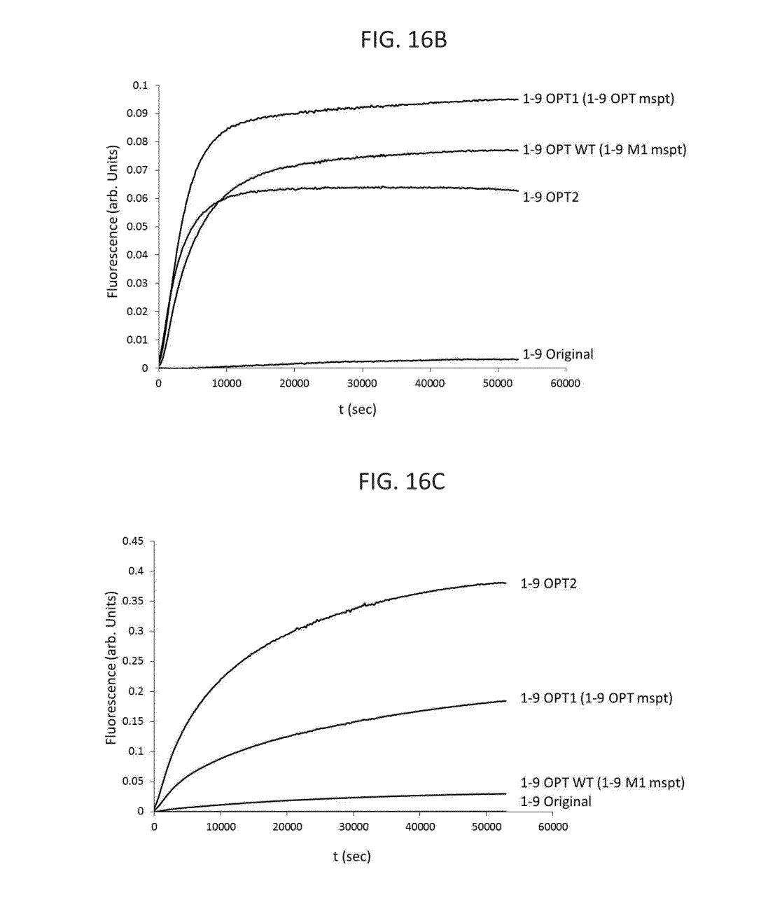

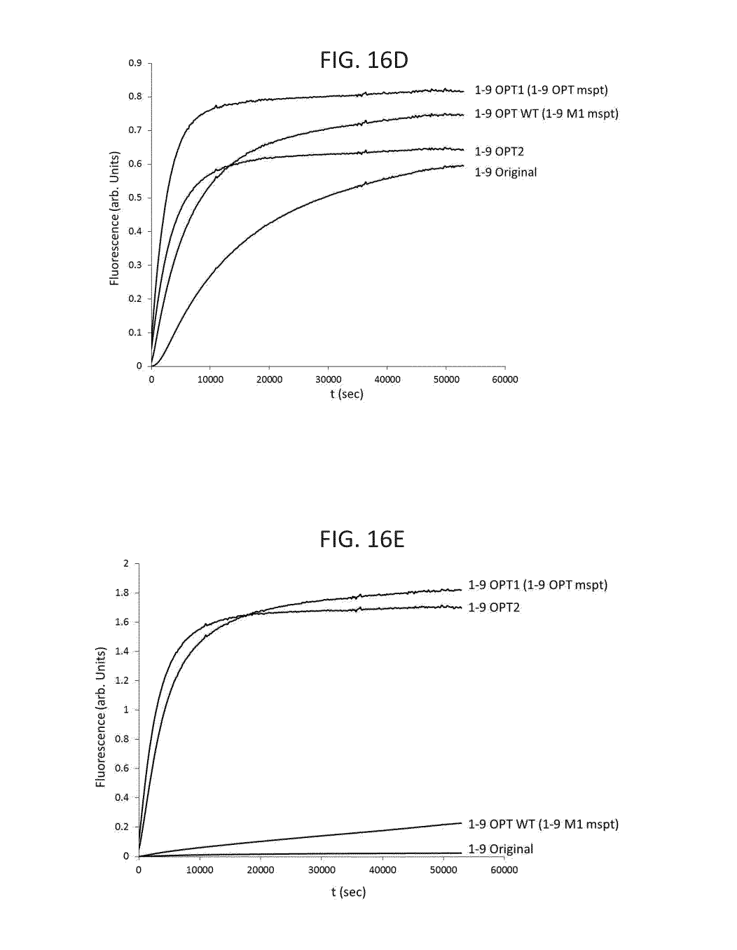

An additional GFP1-9 mutant, GPF1-9 OPT2 (SEQ ID NO: 4), was also identified. As shown in FIGS. 16A-16E, GFP1-9 OPT2 also includes four additional mutations compared to the GFP1-9 WT sequence. Comparison of the GFP1-9 Original, WT, OPT1, and OPT 2 constructs purified from the soluble fraction of E. coli lysate (FIGS. 16B and 16D) or renatured from inclusion bodies (FIGS. C and 16E) and complemented with the GFP10-11 hairpin (FIGS. 16B and 16C) or the 10-SR11 construct (FIGS. 16D and 16E) show that the WT, OPT1, and OPT2 mutants each exhibit unique properties.

It was then examined whether optimized GFP10 and GFP11 (further referred to as GFP10 and GFP11) would affect the solubility of fused proteins. A set of eighteen P. aerophilum test proteins was cloned into a modified pTET vector as GFP10-POI-GFP11 “sandwiches” (POI=protein of interest) to assay solubility levels as previously described (Cabantous, et al., Nat Methods 3, 845-854, 2006). The solubility of these eighteen controls without tags was measured and shown that the GFP11 M3 tag engineered in the earlier study did not perturb passenger solubility (Cabantous, et al., Nat Methods 3, 845-854, 2006). The sandwich fusions using GFP1-10 to detect only the GFP11 tag was first assayed (FIG. 3B). Co-expressing GFP1-10 and the sandwich resulted in bright colonies reflecting total protein expression (FIG. 3B, top). On the other hand, expressing the sandwich first, shutting off expression to allow the proteins to remain soluble or aggregate, then expressing the GFP1-10 detector (sequential induction) gave weak fluorescence for the protein expressed in insoluble form (proteins #6, 8, 15, 16) as expected. This pattern was very similar to that previously seen for the GFP11 M3 tag study (Cabantous, et al., Nat Methods 3, 845-854, 2006) indicating that the GFP10-POI-GFP11 sandwich did not strongly perturb the solubility of the test proteins. Note that the version of GFPS11 developed herein differs from the GFP11 M3 tag described earlier (Cabantous, et al., Nat Methods 3, 845-854, 2006).

Next the complementation of the sandwich with GFP1-9 was studied (FIG. 3B, bottom). Interestingly, whether the sandwich and GFP1-9 were co-expressed or sequentially expressed, only the soluble proteins gave fluorescent colonies (FIG. 3B, bottom). When GFP1-10 is co-expressed, insoluble protein can be labeled (FIG. 3B, top), ostensibly because the available GFP1-10 can capture the GFP11 M3 tag early on, committing the fluorophore to form regardless of the subsequent fate of the fusion. In contrast, even when co-expressed with GFP1-9, one or both tags of insoluble proteins must become inaccessible prior to productive binding with GFP1-9. This suggests the GFP1-9 could be used to stringently assess protein solubility, without the concern of false-positives caused by capturing transiently soluble proteins as with GFP1-10.

Validating a Protein-Protein Interaction Sensor Using Coiled-Coil Heterodimerization.

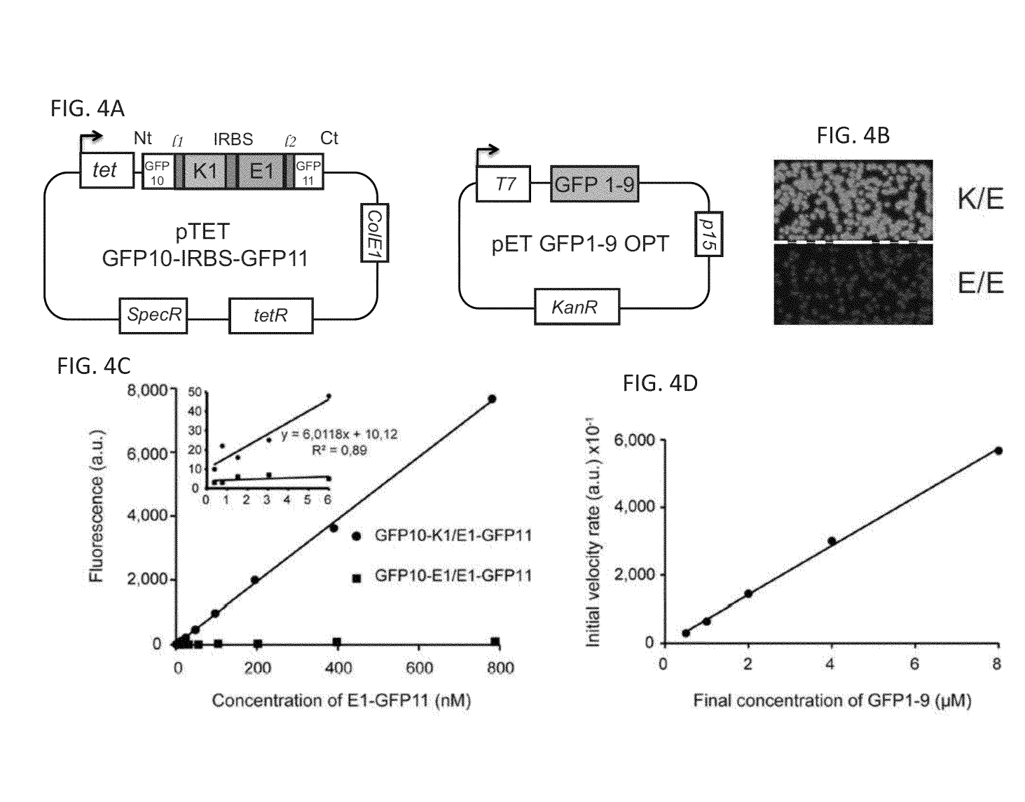

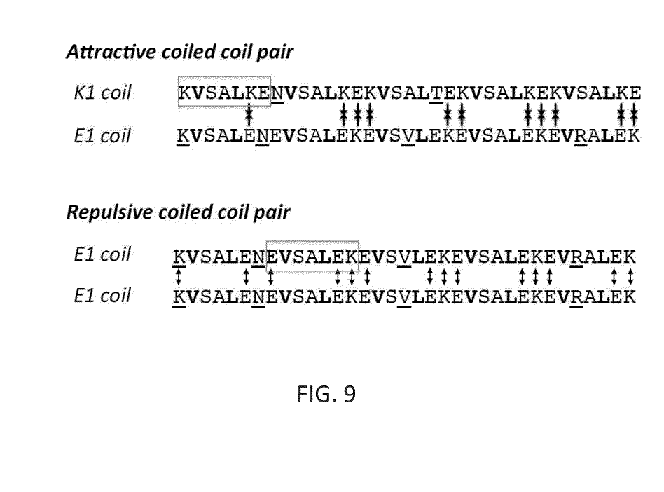

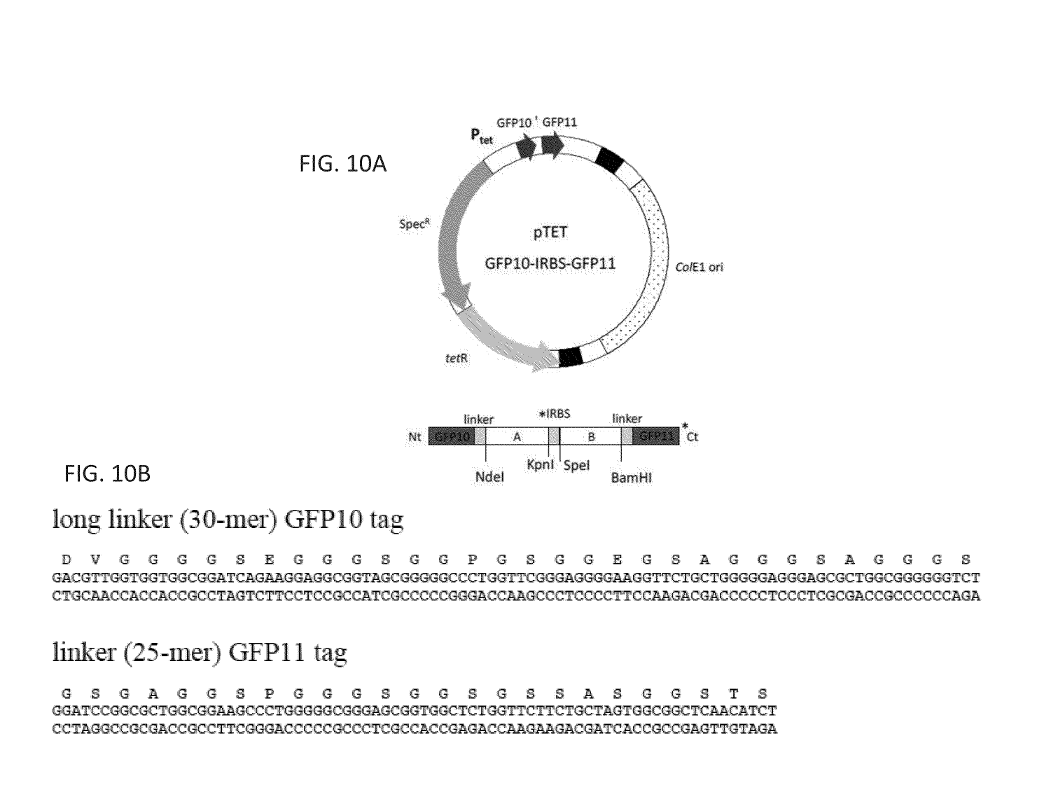

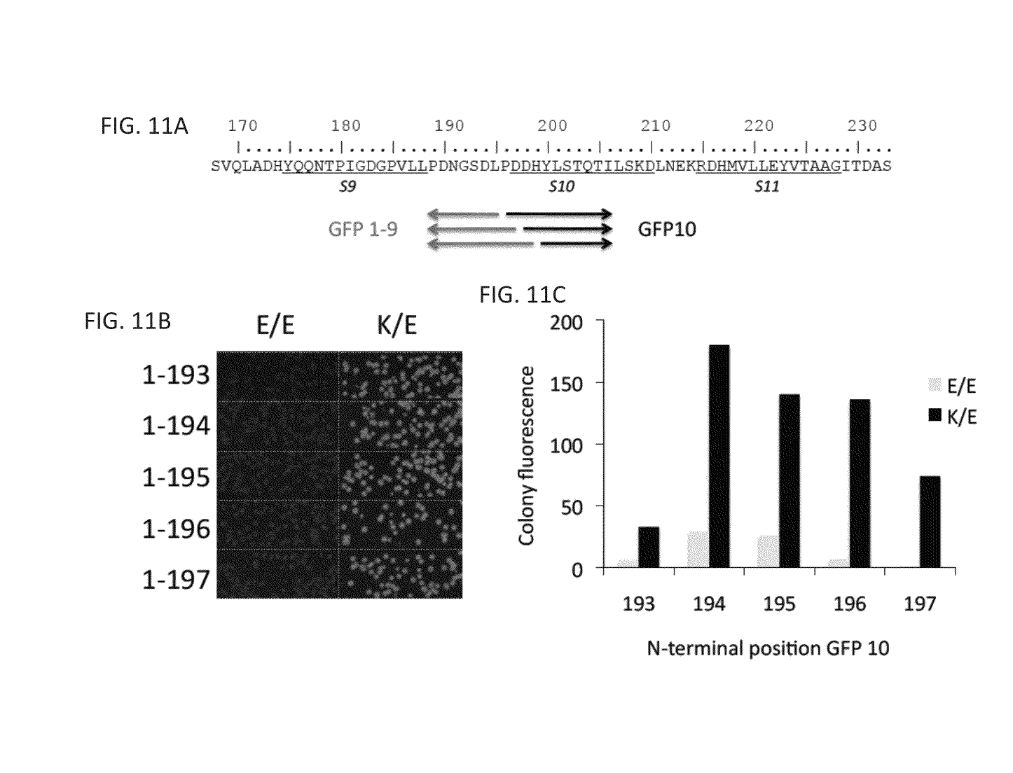

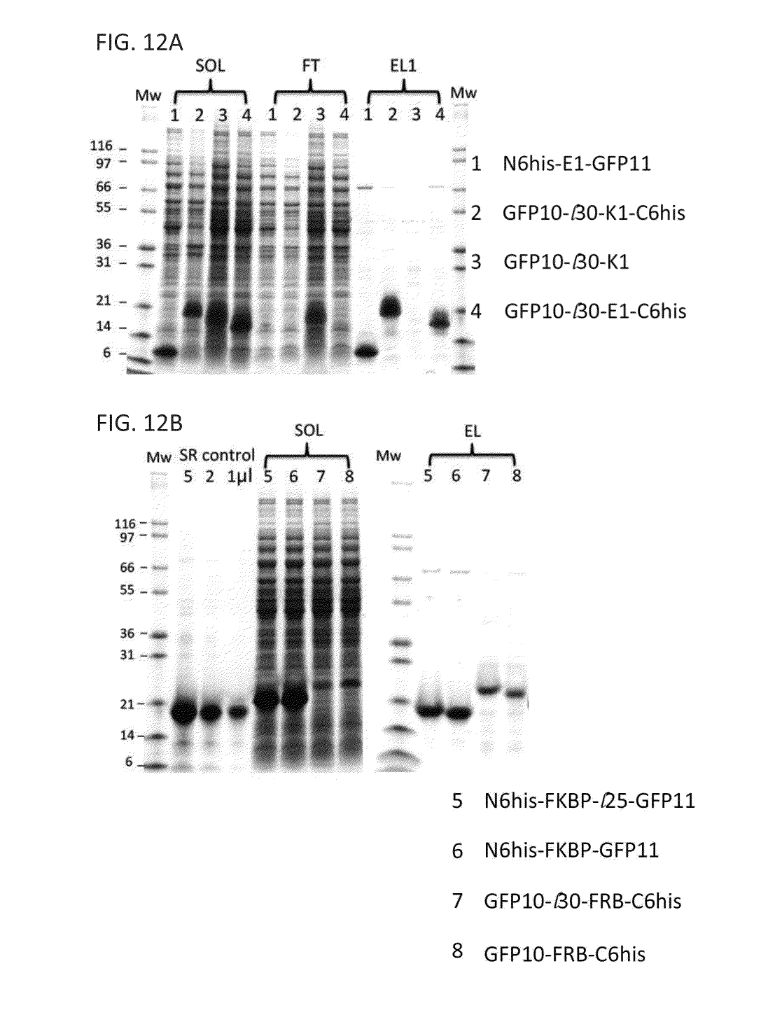

To test whether GFP1-9 is capable of detecting GFP10 and GFP11 fused to separate interacting protein molecules, two pairs of coiled-coils developed by Hodges and coworkers (Tripet, B., et al., Protein Eng 10, 299, 1997) were selected based on their ability to direct preferential interaction between oppositely charged K1/E1 coiled-coils or repulsion for the negatively charged E1/E1 pair (FIG. 9). This model system features rapid association of the K/E heterodimer and a lack of E coil homodimerization (Tripet, B., et al., Protein Eng 10, 299, 1997). Pair-wise combinations of E1-GFP11 and GFP10-E1, or GFP10-K1 and E1-GFP11 were expressed from an anhydrotetracycline (Antet) inducible bicistronic pTET vector (FIG. 10), and transformed into E. coli cells expressing the GFP1-9 from an isopropyl β-D-1-thiogalactopyranoside (IPTG) inducible pET vector (FIG. 4A). After 3 h E. coli GFP1-9 cells co-expressing GFP10-K1 and E1-GFP11 turned brightly fluorescent, thus demonstrating efficient K/E coiled-coil heterodimerization. In contrast, co-expression of GFP10-E1 and E1-GFP11 produced residual background fluorescence, comparable with mock induction after either Antet or IPTG induction in E. coli (FIG. 4B). These results suggest minimal GFP10 and GFP11 self-association in the absence of interacting fused proteins. Using the K1/E1 model, the GFP10 construct (residue 194-212) was selected as the optimal ‘break point’ for detecting protein-protein interactions (FIG. 11). To assess the sensitivity of the assay, GFP10-K1, GFP10-E1, and E1-GFP11 fusion proteins were individually expressed from pET vectors in E. coli and purified using immobilized metal affinity chromatography (FIG. 12A). Quantified GFP10-K1 or GFP10-E1 were mixed with E1-GFP11 at varying concentrations, and supplemented with a molar excess of the GFP1-9 OPT1 fragment (8 μM) to initiate rapidly complementation and ensure that kinetic rate is independent of GFP1-9 concentration. Fluorescence of GFP10-K1/E1-GFP11 interaction assay was linearly related to the concentration of E1-GFP11 substrate, as previously observed with self-assembling split-GFP assays (FIG. 4C; Cabantous, et al., Nat Biotechnol 23, 102-107, 2005). The assay could detect K1/E1 coil interaction down to 1 nM concentration levels, which corresponds roughly to the dissociation constant of the heterodimer (Tripet, B., et al., Protein Eng 10, 299, 1997). As expected, the E1/E1 pair interaction signal remained at a basal level of fluorescence even at very high concentrations (1 mM) (FIG. 4C). These results are completely consistent with a model in which tripartite split-GFP complementation occurs only when GFP10 and GFP11 are brought into close proximity by interacting protein partners, presumably by reduction in entropy. To study how different concentrations of the detecting reagent (GFP1-9) affected the assay, two-fold successive dilutions of GFP1-9 was added to the protein mixtures containing GFP10-K1 and E1-GFP11 at the same final concentration (1 μM each). A slower kinetic complementation rate as the reagent concentration decreased was observed, completely consistent with simple bimolecular reaction kinetics (FIG. 4D). For example, the initial rate for appearance of fluorescence drops by a factor of two each time the concentration of GFP1-9 is halved. Although further studies are needed to understand the precise mechanism of assembly, these observations are consistent with a model in which the coils bring the smaller fragments into proximity prior to detection by GFP1-9, i.e., approximating a two-body system with the tethered GFP10 and GFP11 acting as a module interacting with GFP1-9.

Inducible FRB/FKBP Interactions.

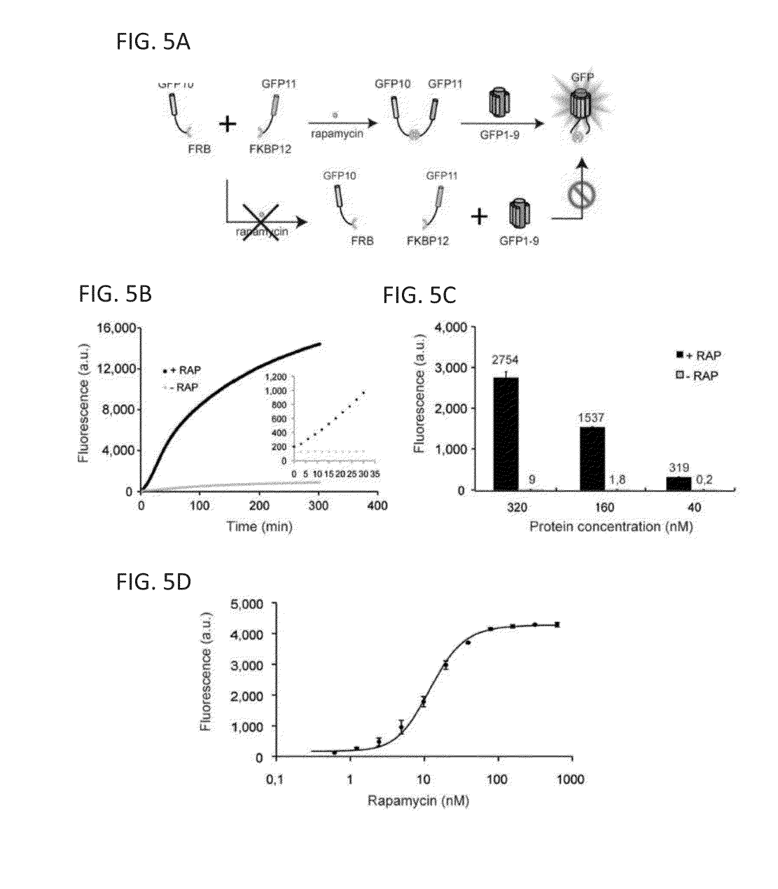

To further test the specificity and dynamic range of the method, the well-studied FKBP12-FRB rapamycin inducible protein interaction (Banaszynski, et al., J Am Chem Soc 127, 4715-4721, 2005) was used. GFP10 was fused to the rapamycin-binding domain of the mammalian target of rapamycin (mTOR) kinase (FRB) and GFP11 to the FK506-binding protein 12 (FKBP) and tested their association in vitro upon addition of recombinant GFP1-9 protein (FIG. 5A). GFP10-FRB and FKBP-GFP11 fusion proteins were co-expressed from bicistronic pTET GFP10-IRBS-GFP11 vector (FIG. 10). In earlier studies of interacting proteins in cell extracts using BiFC, misfolding and aggregation issues interfered with complementation unless the proteins had been co-expressed or were refolded after denaturing (Magliery, et al., J Am Chem Soc 127, 146-157, 2005). In contrast, the soluble crude E. coli cell extracts could be mixed with a 4 μM solution of purified GFP1-9 OPT1 and incubated for several minutes prior to addition of 150 nM rapamycin, after which the rapid increase in fluorescence indicated soluble interacting GFP10-FRB and FKBPO-GFP11 protein. A two-fold increase in fluorescence was easily detectable after only 10 minutes incubation (FIG. 5B, inset), with a half-life of 60 minutes. Without added rapamycin fluorescence remained near blank levels (FIG. 5B). To further examine the behavior of the protein interaction reporter, purified 6-His-GFP10-FRB and 6-His-FKBP-GFP11 fusion proteins were studied (FIG. 12B). In the presence of 150 nM rapamycin (much greater than the reported Kd of ca. 12 nM (Banaszynski, et al., J Am Chem Soc 127, 4715-4721, 2005)), endpoint fluorescence was directly proportional to the amount of added protein complex as expected (FIG. 5C). To further evaluate reaction kinetics and equilibria of rapamycin-FKBP/FRB protein association, the effect of increasing concentrations of rapamycin (0.05 to 300 nM) was tested. The rapamycin dose response curve presents a half maximal value of 13.5 nM, in accordance with previously published data (FIG. 5D; Banaszynski, et al., J Am Chem Soc 127, 4715-4721, 2005).

Monitoring the Association of Protein Complexes in E. coli.

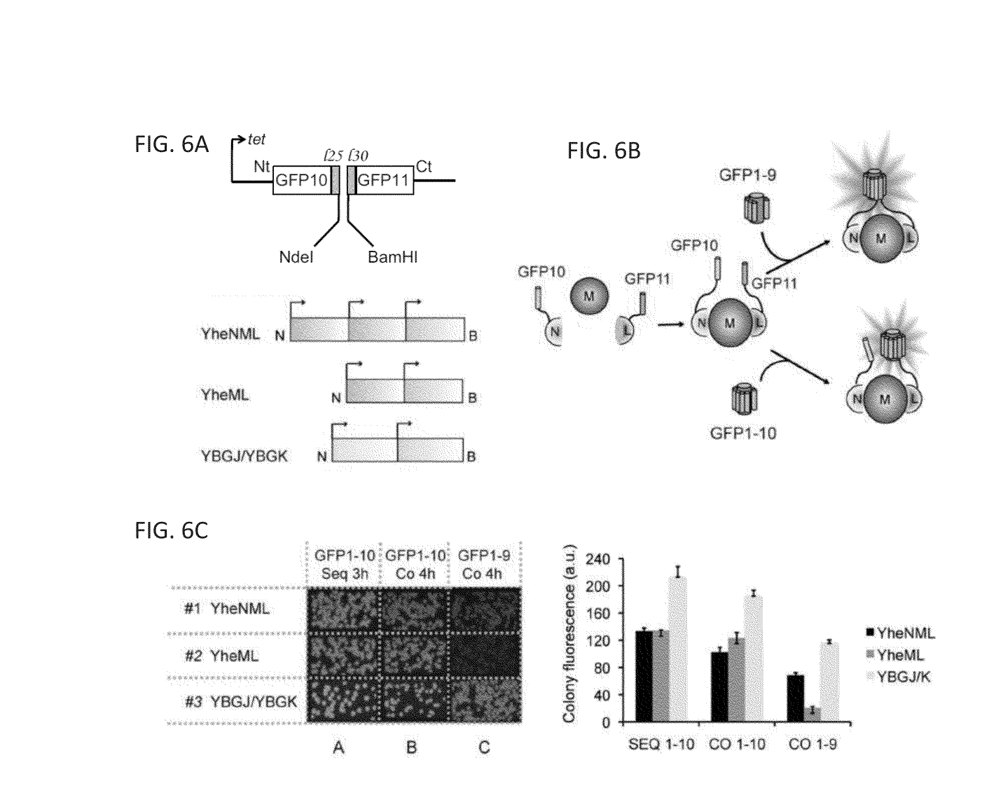

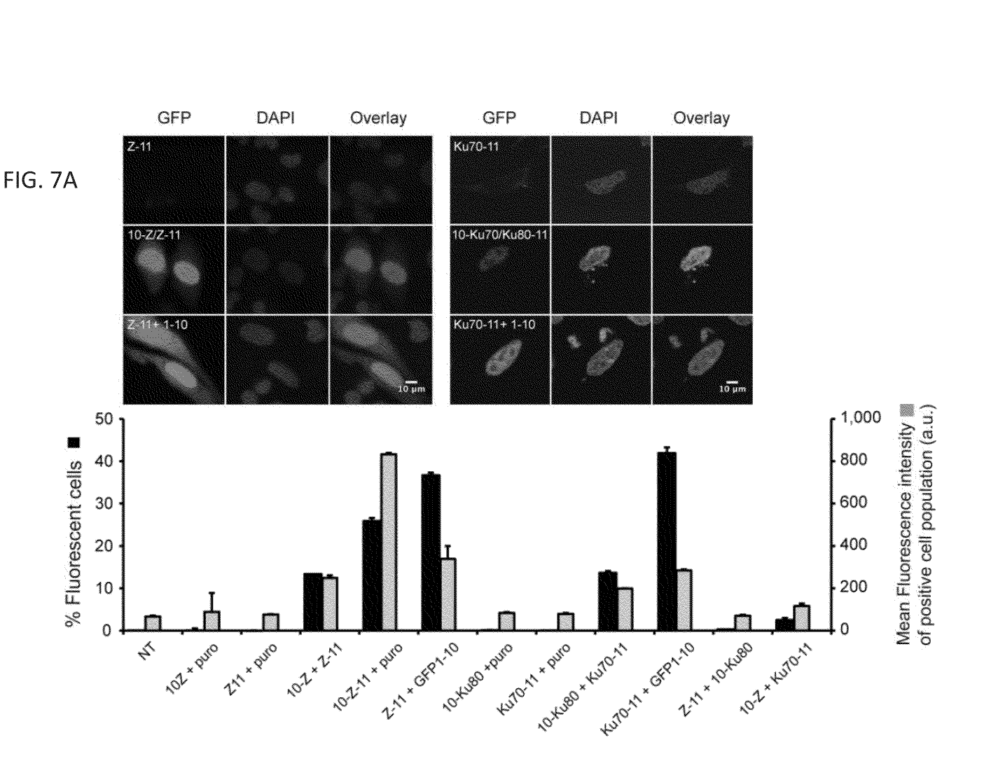

Protein signaling modules often involve more than two interacting partners. To examine the ability of the tagging system to detect multimeric complexes, two polycistrons from the E. coli genome encoding the heterotrimer Tus BCD complex (YheNML; Numata, et al., Structure 14, 357-366, 2006) and a putative allophanate hydrolase constituted by the dimeric assembly YBGK/YBGJ (Lockard, et al., Protein Eng Des Sel 24, 565-578, 2011) were selected. An alternate version of the Tus complex where YheN subunit was omitted, thus preventing stable association of the complex as previously reported (Numata, et al., Structure 14, 357-366, 2006), was also studied. These polycistrons were PCR amplified from genomic DNA including their internal ribosome binding sites. Translation of individual complex subunits was dependent on natural IRBS present in the polycistron, so the first subunit had an N-terminal GFP10 and the last subunit had a C-terminal GFP11 furnished by the pTET SpecR vector (FIG. 6A). To ensure sufficient flexibility between complex subunits, especially for the YheNML whose subunits have nearly buried N termini in the assembled complex (Numata, et al., Structure 14, 357-366, 2006), 30 and 25-mer linkers were inserted between the polycistron and the split-GFP tags (FIG. 6B and FIG. 10). These constructs were transformed into E. coli cells expressing either the GFP1-9 OPT1 fragment for complex detection (requiring both the GFP10 and GFP11 tag), or the GFP1-10 fragment (Cabantous, et al., Nat Methods 3, 845-854, 2006) to assess the expression level and solubility by monitoring the GFP11 tag (FIGS. 6B and 6C). All the complexes were well expressed and highly soluble as shown in FIG. 6C, column A, B. Cells expressing the trimeric YheNML complex became fluorescent after 4 h complementation with GFP1-9 OPT1, as expected (FIGS. 6C, 1D). In contrast, weak fluorescence was seen for colonies expressing only YheM and YheL (missing the YheN subunit) with GFP1-9 OPT1 (FIGS. 6C, 2D). Colonies expressing the YBGJ/YBGK control complex were brightly fluorescent after complementation with GFP1-9 OPT1 as expected (FIGS. 6C, 3D). These results are in good agreement with those obtained in previous bead-based assays using GFP1-10 complementation (Lockard, et al., Protein Eng Des Sel 24, 565-578, 2011).

Visualization of Protein-Protein Interactions in Mammalian Cells.

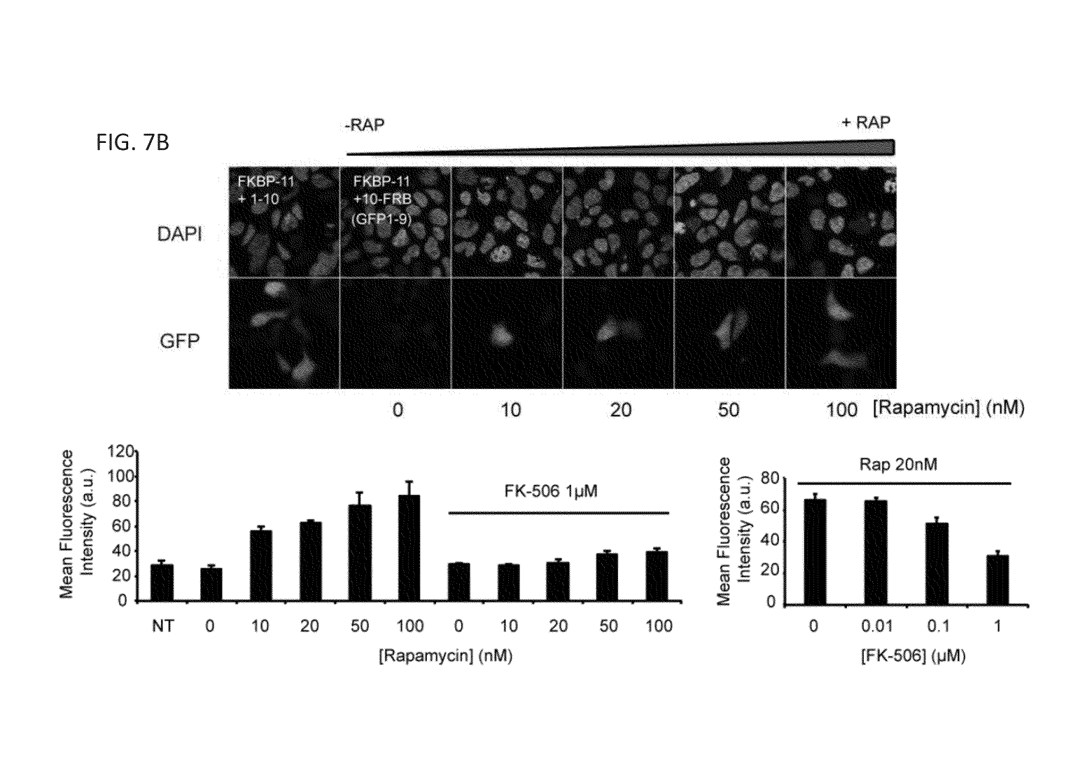

The split-GFP fragments were adapted for optimized expression in mammalian cells and tested the formation of several eukaryotic complexes by fluorescence microscopy. As proof of principle, the yeast GCN4 leucine zipper (Remy, et al., Nat Methods 3, 977-979, 2006) was fused to each of the GFP10 and GFP11 tags and co-transfected both constructs along with the GFP1-9 detector fragment into CHO cells to study the formation of GCN4 heterodimer (FIG. 7A, left panel). Nuclear fluorescence corresponding to leucine zipper heterodimerization was observed in cells expressing GFP1-9 after 24 h of transfection (FIG. 7A, lane 2). As expected, co-expression of GFP1-9 and one of the leucine zipper domain C-terminally fused to GFP11 did not produce fluorescence (FIG. 7A, lane 1). In a separate experiment, the fluorescence complementation of the GFP11 tagged proteins with GFP1-10, which measures soluble protein levels in cells (Cabantous, et al., Nat Biotechnol 23, 102-107, 2005), was observed. As expected, the leucine zipper domain alone was strongly localized in the nucleus and only weakly in the cytoplasm (FIG. 7A, lane 3). Much larger protein complexes, such as the Ku70-Ku80 complex (Cary, et al., Nucleic Acids Res 26, 974-979, 1998) that is required in the non-homologous end-joining (NHEJ) pathway in DNA repair, were also detected. Heterodimerization between GFP10-Ku80 and Ku70-GFP11 in HEK 293 cells expressing stably the GFP1-9 OPT1 fragment was also detected (FIG. 7A, right panel). To shed additional light on possible background from the tripartite assay in living cells, leucine-zipper and Ku proteins that localize in the same sub cellular compartment were co-expressed. Quantification of the fluorescence levels by flow activated cell sorting (FACS) indicated basal levels of fluorescence from individual split-GFP controls and between non-interacting proteins tested. Pairwise leucine zipper and Ku subunit heterodimerization led to fluorescence levels comparable to the bipartite split-GFP assay, which titrates soluble protein levels in cells (FIG. 7A; Cabantous, et al., Nat Biotechnol 23, 102-107, 2005). In parallel, GFP10-FRB and FKBP-GFP11 protein fusions were co-expressed in HEK 293_GFP1-9 cells, stimulated with increasing concentrations of rapamycin. Fluorescence levels for the unstimulated cells remain basal, whereas bright fluorescent cells could be visualized with as little as 10 nM rapamycin, in the range of the EC₅₀ value measured for the ternary complex in vitro (FIG. 7B; Marz, et al., Mol Cell Biol 33, 1357-1367, 2013). Moreover, pre-addition of an excess of competitive inhibitor FK-506 totally prevented FRB/FKBP association by rapamycin in an FK-506 dose dependent manner (FIG. 7B, bottom panel), thus demonstrating that tripartite split-GFP complementation can be used to monitor inhibitors of protein-protein interactions by small molecule compounds prior to association of interacting partners.

### Discussion

This example provides the first description of a protein-protein interaction reporter based on tripartite split-GFP association. Instead of the bulky and poorly folded BiFC fragments of GFP (Hu, et al., Mol Cell 9, 789-798, 2002; Ghosh, et al., J. Am. Chem. Soc. 122, 5658-5659, 2000), the tags based on small engineered β-strands of GFP (˜20 amino acids long) minimize protein interference and aggregation. Interaction assays using the E-coil/K-coil model (Tripet, B., et al., Protein Eng 10, 299, 1997) have a sensitivity limit in the picomole range. Chemically induced interactions of the FRB/FKBP complex are detectable using the split-GFP within a few minutes after addition of rapamycin in vitro. Bulky BiFC fragments from various fluorescent proteins are expressed largely in E. coli in inclusion bodies (Magliery, et al., J Am Chem Soc 127, 146-157, 2005), and the effect of the fragments from other enzyme-based PCA assays on the folding of fused proteins is poorly understood (Ozawa, et al., Anal Chim Acta 556, 58-68, 2006). The tagging system is highly soluble, permitting production of high yield of fusion proteins at 37° C. in E. coli. The assay is therefore not temperature dependent, unlike other BiFC for which co-expression and decreasing growth temperature, or refolding denatured extracts are the only ways to improve assembly of split-GFP fragments (Magliery, et al., J Am Chem Soc 127, 146-157, 2005; Robida, et al., J Mol Biol 394, 391-409, 2009). The method is sensitive enough to detect association of proteins expressed from E. coli polycistronic mRNAs, and fluorescence is correlated with independently measured protein complex formation and stability. As shown for the TusABC (YheNML) studies above, the formation of larger trimeric complexes by extending the length of the linkers between the GFP tag and the protein of interest can be monitored. This suggests that precise geometry of the GFP10 and GFP11 is not critical for interaction with GFP1-9, but rather that reduction of entropy is important for triggering assembly of the GFP10, GFP11, and GFP1-9.

The newly described tripartite split-GFP assay is a promising tool to study protein-protein interactions in vitro and in living cells. Its chief advantage over the bulky aggregation-prone fragments of existing BiFC is the small sizes of the GFP10 and GFP11 tagging peptides. It is therefore well suited to study interactions of unstable protein complexes that are difficult to detect with larger GFP tags. The disclosed system greatly expands the prospect for protein-interaction screening and the design of new biosensors for protein complex assembly and association (Kellermann, et al., Chembiochem 14, 200-204, 2013). Although association of the 3-body GFP fragments is currently irreversible, this system can be exploited to turn on the detection of protein complex formation by simple addition of GFP1-9 reagent. The technology should prove useful for high-throughput interaction screens of libraries of protein-protein interfaces and domains (Pedelacq, et al., Nucleic Acids Res, 2011), the study of complex formation stability, and the robust detection of soluble proteins and protein complexes by flow cytometry. The technology should prove useful for screening small molecule compounds, for example inhibitors for the interfaces of macromolecular protein complexes. Further work might yield split tripartite GFPs whose assembly can be regulated with light (Do, et al., J Am Chem Soc 133, 18078-18081, 2011).

### Methods

Cloning.

K1-coil and E1-coil DNA, FRB (NM_{—}004958) and FKBP (NM_{—}000801) sequences were amplified by PCR using synthetic oligonucleotides and cloned into a pTET ColE1 SpecR GFP10/11 via NdeI:KpnI (GFP10 fusion) or SpeI:BamHI (GFP11 fusion). Linker extension of pTET GFP10/11 was performed using inverse PCR with synthetic oligonucleotides (FIG. 10). GFP1-9 was cloned into a pET28a p15 Kan vector via NdeI:BamHI sites. Proteins expressed with GFP10 (E1 and K1 coils, FRB) and proteins expressed with GFP11 (E1 coil, FKBP) were subcloned into a pET vector bearing a C-terminal or N-terminal 6-His tag respectively, prior to purification and characterization. For mammalian expression, all the constructs used in the study were derived from pcDNA 3.1 Zeo vector backbone encoding GCN4-hGLuc1 and hLuc2-GCN4. Split-luciferase domains were replaced with GFP10 and GFP11 fragments. Ku80, FRB were cloned into BspeI:XbaI sites of pcDNA_GFP10; Ku70 and FKBP were amplified from cDNA and inserted into NotI:ClaI cloning sites of pcDNA_GFP11 vector.

The oligonucleotides used to amplify E. coli polycistrons were as follows:

Forward NdeI primer to YBGJ of YBGJ/YBGK E. coli:
(SEQ ID NO: 16)
AGATATACATATGCAACGAGCGCGTTGTTATCTGATAGG
Bottom BamHI non-stop to YBGK of YBGJ/YBGK E. Coli:
(SEQ ID NO: 17)
AATTCGGATCCATTTTCATTGTGCAGCCGCCACGCTA
Forward NdeI primer to YheN E. coli:
(SEQ ID NO: 18)
AGATATACATATGCGTTTTGCCATCGTGGTGACCGGGCC
Forward NdeI primer to YheM E. coli:
(SEQ ID NO: 19)
AGATATACATATGAAACGAATTGCGTTTGTTTTTTCTAC
Reverse BamHI primer to YheL E coli:
(SEQ ID NO: 20)
AATTCGGATCCCCAGGCCATCTGGCTGGAGTGCTTAA

Directed Evolution of Split-GFP Fragments for Tripartite Complementation.

The DNA construct encoding (GFP10)-L1(DVGSGGGS, SEQ ID NO: 21)-NdeI::(GGGSGSGG, SEQ ID NO: 22))::BamHI-L2(GGGSGGGS, SEQ ID NO: 23)-(GFP11), where GFP10 and GFP11 sequences were derived from superfolder GFP, was evolved by DNA shuffling. Libraries of GFP10-11 variants expressed from pTET SpecR vector were screened for improved solubility using a sequential induction protocol (Cabantous, et al., Nat Methods 3, 845-854, 2006) in E. coli cells containing GFP1-9 M1 on a pET p15 vector. At each round, protein solubility of selected optima was verified by complementation with GFP1-10 in vitro. From the six brightest clones sequenced after three rounds of evolution, one best mutant (#5) was identified, termed GFP11 M4 (FIG. 13). Upstream GFP10 fragment (GFP10 M1) was further evolved by DNA shuffling and primer doping mutagenesis with a pool of fourteen synthetic oligonucleotide primers. Each primer was centered at one of the fourteen amino acids of the GFP10 M1 domain, containing an NNN coding degeneracy at the central target amino acid and flanking homology to the GFP10 M1 in the context of the cloning vector. A partially soluble protein HPS was then inserted in the NdeI:BamHI cloning site to obtain (GFP10 mutant M1)-L1-HPS-L2-(GFP11 mutant M1) in pTET SpecR vector. After three rounds of selection using the sequential induction format from the pTET and pET plasmids (Cabantous, et al., Nat Methods 3, 845-854, 2006), one best-performing clone, termed (GFP10 M2)-L1-Nde1::HPS::BamH1-L2-(GFP11 M4) was isolated. GFP10 M2-HPS-GFP11 M4 fusion and GFP1-9 M1 inserts were swapped between pTET and pET plasmids to perform directed evolution of GFP1-9 M1. Briefly, cDNAs libraries of GFP1-9 were expressed from pTET SpecR plasmids and tested for in vivo complementation assays in an E. coli strain expressing GFP10 M2-HPS-GFP11 M4 fusion in pET p15. At the second round, GFP1-9 OPT1 was isolated and sequenced.

GFP1-9 In Vitro Assays.

Production of recombinant GFP1-9 OPT1 protein fragment was performed according to previous protocol described for split-GFP1-10 (Cabantous, et al., Nat Methods 3, 845-854, 2006). GFP1-9 was expressed from a pET p15 vector without a 6H is tag and produced after induction 1 mM IPTG for 4 hours at 37° C. The protein was refolded from washed inclusions bodies (see GFP1-10 protocol; Cabantous, et al., Nat Methods 3, 845-854, 2006) and solubilized in TNG buffer. To perform kinetic characterization of the newly engineered GFP1-9 OPT1 and GFP1-9 M1, 180 μl of equal amounts of GFP1-9 refolded pellet fractions (ca. 0.5 mg/ml) were mixed with 20 μl of a soluble protein control sulfite reductase (SR) in fusion with sandwich GFP10 and GFP11 (GFP10-SR-GFP11) or GFP10-11 peptide (3.5 μM each). For K1/E1 and E1/E1 kinetic saturation studies, 25 μl of E1-GFP11 (6.25 μM) were mixed with 50 μl of GFP10-K1 or GFP10-E1 at various concentrations (800 nM down to 0.4 nM). For ligand induced interactions, rapamycin (LC laboratories) was diluted in DMSO and added in 10 μl aliquot to the final FRB/FKBP protein assay mix 20 μl of GFP10-FRB+20 μl FKBP-GFP11 (0.5 mg/ml). Assay was repeated by diluting FRB/FKBP samples in TNG buffer. Complementation was induced by addition of a large excess of GFP1-9 OPT1 (150 μl, 0.25 mg/ml). Fluorescence kinetic (λ_{exc}=488 nm/λₑₘ=530 nm) was monitored with a FL600 Microplate Fluorescence Reader (Bio-Tek), at 3 min intervals, for 15 h. The background fluorescence of a blank sample (150 μl of GFP1-9 OPT1, 0.25 mg/ml and 50 μl 0.5% (w/v) BSA in TNG buffer) was subtracted from the final fluorescence values.

Mammalian Cell Interactions Assays.

CHO cells were grown in Ham's F-12 medium (Gibco, Invitrogen Co) and 10% (v/v) fetal bovine serum (FBS); HEK 293 cells were grown in Dulbecco's Modified Eagle Medium (DMEM) and 10% (v/v) FBS. CHO cells were co-transfected with Lipofectamine 2000 (Gibco, Invitrogen Co.) with plasmids encoding for GFP1-9 OPT1, GCN4-GFP11, GFP10-GCN4 or GCN4-GFP11 M4+ GFP1-10. Stable GFP1-9 cell lines were produced by lentiviral transduction of HEK 293. HEK 293_GFP1-9 cells were co-transfected with jetPRIME reagent (Polyplus transfection) with 0.5 μg of each plasmid (GFP10 and GFP11 fusions); Twenty-four hours after transfection, cells were washed with PBS and mounted in DAPI-containing ProLong Gold antifading reagent (Molecular Probes). Imaging was performed using a LEICA DM-RB fluorescence microscope, with a 40× and 100× oil immersion objective to visualize the stained cells. Images from CHO cells were acquired with a PHOTOMETRIC™ COOLSNAP HQ™ camera and analyzed with Metamorph or ImageJ software. Imaging of HEK cells was performed using a Zeiss (Carl Zeiss) confocal laser scanning inverted microscope (LSM 710 NLO with Quazar spectral detector array). Flow cytometry measurements were performed using a BD Biosciences FACSCalibur™ cytometer. Data analysis was performed using CellQwest® software (BD Biosciences).

### Example 2

### Split Green Fluorescent Protein as a Modular Binding Partner for Protein Crystallization

A modular strategy for protein crystallization using split green fluorescent protein (GFP) as a crystallization partner is provided. Insertion of a hairpin containing GFP beta-strands 10 and 11 into a surface loop of a target protein provides two chain crossings between the target and the reconstituted GFP compared with a single connection afforded by terminal GFP fusions. This strategy is tested by inserting this hairpin into a loop of another fluorescent protein, sfCherry. The crystal structure of the sfCherry-GFP10-11 hairpin in a complex with GFP1-9 was determined at a resolution of 2.6 Å. An analysis of the complex shows that the reconstituted GFP is attached to the target protein (sfCherry) in a structurally ordered way. This work opens the way to rapidly creating crystallization variants by reconstituting a target protein bearing the GFP10-11 hairpin with a variety of GFP1-9 mutants engineered for favorable crystallization.

### Introduction

Structural characterization of proteins, protein complexes and small molecules is essential to understand cellular functions from enzymology to macromolecular machines. Knowledge of protein structures has led to re-design of protein function and folding using rational and semi-rational approaches, and has promoted the discovery of new and improved small molecule drugs (Lu, et al., Nature 460, 855-862, 2009; Yeung, et al., Nature 462, 1079-1082, 2009; Lin, et al., Neoplasia 12, 39-50, 2010). Yet obtaining well-ordered crystals, a prerequisite of macromolecular crystallography, remains a major obstacle; as many as 70% of purified proteins fail to crystallize (Terwilliger, et al., Annu. Rev. Biophys. 38, 371-383, 2009).

A number of current approaches to improve protein crystallization involve constructing variant forms of the target protein molecule. Examples include engineering proteins with enhanced solubility using site-directed mutagenesis (Nasreen, et al., Protein Sci. 15, 190-199, 2006; Eichinger, et al., J. Biol. Chem. 282, 31068-31075, 2007) or directed evolution (Farinas, et al., Curr. Opin. Biotechnol. 12, 545-551, 2001; Pedelacq, et al., Nature Biotechnol. 20, 927-932, 2002; Waldo, Curr. Opin. Chem. Biol. 7, 33-38, 2003; Cabantous, et al., J. Struct. Funct. Genomics 6, 113-119, 2005); removal of disordered regions, often at the N or C-terminus (Thornton, et al., J. Mol. Biol. 167, 443-460, 1983) by proteolysis (Dong, et al., Nature Methods 4, 1019-1021, 2007) or targeted deletion (Pantazatos, et al., Proc. Natl. Acad. Sci. USA 101, 751-756, 2004) based on disorder prediction. Proteins may also contain internally disordered regions such as loops or subdomains, which can sometimes be removed, shortened or replaced with a short linker to reduce conformational heterogeneity, thereby increasing crystallization propensity (Kwong, et al., Nature 393, 648-659, 1998; Kwong, et al., J. Biol. Chem. 274, 4115-4123, 1999; Derewenda, Acta Cryst. D66, 604-615, 2010). Other methods such as surface entropy reduction (Longenecker, et al., Acta Cryst. D57, 679-688, 2001; Derewenda, Structure 12, 529-535, 2004; Cooper, et al., Acta Cryst. D63, 636-645, 2007) and lysine methylation (Rypniewski, et al., Biochemistry 32, 9851-9858, 1993; Walter, et al., Structure 14, 1617-1622, 2006; Kim, et al., Nature Methods 5, 853-854, 2008) drive crystallization by changing the surface properties of proteins and promoting lattice contacts. The surface entropy reduction method has been successfully applied to not only individual proteins but also protein-protein complexes and membrane proteins (Berman, et al., Nucleic Acids Res. 35, D301-303, 2007; Levinson, et al., Cell 134, 124-134, 2008; Yanez, et al., J. Mol. Biol. 375, 471-486, 2008; Pornillos, et al., Cell 137, 1282-1292, 2009; Yip, et al., Nature 435, 702-707, 2005).

Other methods for modifying and potentially improving the crystallization properties of a protein involve connecting it to another protein, intended to act as a carrier. Highly soluble proteins have been used as fusion partners to the N-terminus or C-terminus of proteins to enhance their folding and solubility and to mediate crystal contacts (Wiltzius, et al., Protein Sci. 18, 1521-1530, 2009; Kuge, et al., Protein Sci. 6, 1783-1786, 1997; Center, et al., Protein Sci. 7, 1612-1619, 1998; Monne, et al., Nature 456, 653-657, 2008; Ullah, et al., Protein Sci. 17, 1771-1780, 2008; Smyth, et al., Protein Sci. 12, 1313-1322, 2003; Moon, et al., Protein Sci. 19, 901-913, 2010). Carrier proteins have been inserted into loops of transmembrane proteins (Engel, et al., Biochim Biophys Acta 1564, 38-46, 2002), and insertion of T4 lysozyme into a loop of the 132-adrenergic receptor is an example of a successful application of that strategy (Rosenbaum, et al., Science 318, 1266-1273, 2007; Cherezov, et al., Science 318, 1258-1265, 2007). Non-covalent crystallization chaperones such as Fab and Fv fragments of antibodies (Kovari, et al., Structure 3, 1291-1293, 1995; Lange, et al., Proc. Natl. Acad. Sci. USA 99, 2800-2805, 2002; Lee, et al., Proc. Natl. Acad. Sci. USA 102, 15441-15446, 2005; Ostermeier, et al., Nature Struct. Biol. 2, 842-846, 1995; Monroe, et al., J. Struct. Biol. 174, 269-281, 2011) and designed ankyrin repeat protein (DARPin) (Monroe, et al., J. Struct. Biol. 174, 269-281, 2011) have been alternatively used to produce complexes with target molecules. These complexes often show improved solubility and crystallizability in comparison to the isolated targets (Derewenda, Acta Cryst. D66, 604-615, 2010).

Synthetic symmetrization of proteins offers a further approach to expand crystallization opportunities. Variant forms of a target protein molecule are constructed, with each designed to produce a structurally distinct oligomer. Disulfide-based synthetic dimerization (Banatao, et al., Proc. Natl. Acad. Sci. USA 103, 16230-16235, 2006; Forse, et al., Protein Sci. 20, 168-178, 2011) and designed metal-mediated oligomerization have both been demonstrated (Laganowsky, et al., Protein Sci. 20, 1876-1890, 2011). Other examples using different motifs such as leucine zippers to drive self-association of a target protein have also been shown to promote protein symmetrization and crystallization (Yamada, et al., Protein Sci. 16, 1389-1397, 2007).

With current strategies for expanding the crystallization opportunities for a target protein, the effort required to produce many structural variants is a major challenge. A modular approach could offer important advantages. In particular, an ideal strategy might factor the problem of repeatedly re-engineering a protein of interest into two separate problems: (1) connecting the target protein to a carrier protein, and (2) creating variant forms of the carrier protein. In order to fully separate the two problems, the connection between the target protein and the carrier protein should occur by non-covalent molecular recognition rather than by genetic covalent attachment, so that repeated genetic modification and purification of the protein of interest can be avoided. Additionally, the target protein and the carrier protein should ideally be attached in a way that minimizes flexibility between them, as too much flexibility would reduce the chances of forming well-ordered crystals of the complex. Finally, the structural feature that drives the non-covalent association between the target protein and the carrier protein should ideally be transferable from one target system to another. In that way, one set of variational forms of the carrier protein can be utilized, without continual re-engineering, for a range of target proteins.

In this work, a system that meets the design requirements above and is based on Green Fluorescent Protein (GFP) is demonstrated. GFP has been employed before in crystallization experiments based on protein fusions (Suzuki, et al., Acta Cryst. D66, 1059-1066, 2010). Also, previous studies on GFP have shown that it can form the basis for a complementation system: fragments composed of either beta strand 11 or a hairpin comprised of beta strands 10 and 11 can reassemble with truncated forms of GFP lacking those segments (Cabantous, et al., Nature Biotechnol. 23, 102-107, 2004). It is shown here that beta strands 10 and 11 of GFP can be inserted as a hairpin into a protruding loop of a target protein, which when complemented by GFP1-9 gives rise to a well-ordered complex, with two polypeptide chain crossings between the two components, which is amenable to crystal structure analysis. Prospects for developing the system for general applications are discussed.

### Methods

Engineering Superfolder Cherry.

The monomeric fluorescent protein Cherry (Shaner, et al., Nature Biotechnol. 22, 1567-1572, 2004) was cloned as a C-terminal fusion to ferritin in a modified pET expression plasmid as described (Pedelacq, et al., Nature Biotechnol. 24, 79-88, 2006). The N- and C-terminal GFP sequence extensions (residues MVSKG (SEQ ID NO: 24) and MDELYK (SEQ ID NO: 25), respectively that were added to improve mCherry protein solubility in an earlier study (Shaner, et al., Nature Biotechnol. 22, 1567-1572, 2004) were omitted here to increase the stringency of selection for better solubility and stability. The DNA encoding mCherry was amplified by PCR using vector flanking primers and was subjected to DNA fragmentation and shuffling using published protocols (Stemmer, Proc. Natl. Acad. Sci. USA 91, 10747-10751, 1994). The cDNA library plasmid pool was transformed into E. coli BL21 (DE3) gold (Novagen) competent cells for protein expression. The library was plated on nitrocellulose membranes using two sequential 400-fold dilutions of a 1.0 OD₆₀₀ₙₘ cell stock frozen in 20% glycerol/Luria-Bertani (LB), yielding ˜3×10³ colonies per plate. Cells were grown overnight at 32° C. and proteins were expressed by transferring the membrane to an LB/agar plate containing 50 mg kanamycin/ml media and 1 mM isopropyl-b-D-thiogalactopyranoside (IPTG) for 3 hours at 37° C. Clones displaying the brightest fluorescence (550 nm excitation/610 nm emission) were selected, grown overnight and frozen in 20% glycerol/LB freezer stocks at −80° C. These brightest clones were selected as templates for the next round of evolution. After three rounds of directed evolution, sequences of constructs were confirmed by DNA sequencing and the brightest clone coding for sfCherry was chosen.

Insertion of GFP strands 10-11 hairpin and selection of clone with permissive loop. GFP strands 10-11 (DLPDDHYLSTQTILSKDLNEKRDHMVLLEYVTAAGITDAS, SEQ ID NO: 26 with residues in strand 10 and strand 11 in bold and the 3-residue linker DAS (italicized)) were inserted into permissive loops of sfCherry by PCR. Fragments were cloned into pTET ColE1 vector and transformed into BL21 (DE3) competent cells containing pET GFP strands 1-9 for in-vivo testing. In-vivo protein expression and solubility screenings were performed as previously described (Cabantous, et al., Nature Methods 3, 845-854, 2006). 1 OD_{600 nm} frozen cell stocks in 20% glycerol/LB were thawed and diluted 400-fold (twice) in LB and plated onto a nitrocellulose membrane with selective LB-agar containing 35 μg/ml kanamycin and 75 μg/ml spectinomycin. After overnight growth at 32° C., the membrane was transferred to a pre-warmed plate containing 250 ng/ml anhydrotetracycline (AnTet), 1 mM IPTG for 4 hours at 30° C. for protein expression screening. For protein solubility testing, membrane was transferred to a pre-warmed plate containing 250 ng/ml AnTet for 2 hours, rested back to its original LB-Kan-Spec plate for 1 hour to allow the AnTet to diffuse out, and followed by induction on an LB-Kan-Spec plate with 1 mM IPTG at 30° C. for 1 hour. The induced plates were illuminated using an Illumatool Lighting System (LightTools Research) equipped with a 488 nm/520 nm (for GFP) and 550 nm/610 nm (for sfCherry) excitation/emission filters.

Expression and Refolding of GFP1-9 and GFP1-10 Fragments.

GFP1-9 and GFP1-10 proteins were expressed and prepared as previously described (Cabantous, et al., Nature Biotechnol. 23, 102-107, 2004; Cabantous, et al., Nature Methods 3, 845-854, 2006). Briefly, 1 liter cultures of BL21(DE3) cells expressing GFP1-9 or GFP1-10 constructs were grown until an OD^{600nm} of 0.5-0.7 was reached, protein expression was induced with 1 mM IPTG and cells were harvested after 5 hours of induction at 37° C. The harvested cells were resuspended in 50 mM Tris pH 7.4, 0.1M NaCl, 10% glycerol (TNG buffer) and lysed by sonication on ice. Inclusion bodies containing GFP1-10 and GFP1-9 were recovered by centrifugation at 20,000 g. Inclusion bodies were washed and prepared in individual EPPENDORF™ tubes (—75 mg inclusion bodies/tube) as previously described (Cabantous, et al., Nature Methods 3, 845-854, 2006). Prepared inclusion bodies can be stored at −80° C. for at least several months. 75 mg of the washed inclusion body prepared in 1.5 ml tube was unfolded with 1 ml of 9M urea in TNG buffer and refolded by adding 25 volumes of TNG buffer. The soluble solutions were filtered through a 0.2 mm syringe filter and protein was quantified using the BIO-RAD PROTEIN ASSAY REAGENT™ (Bio-Rad). This refolded protein solution is ready for protein complementation and can also be stored up to a week at −20° C. for later use.

Expression and Purification of sfCherry and sfCherry-GFP10-11/GFP1-9 Complex.

sfCherry with GFP strands 10-11 inserted at position D169/G170 was sub-cloned into pET with a non-cleavable C-terminal His₆ tag. The C-terminal in-frame BamHI site introduced a GS amino acid motif between sfCherry and the His₆ tag. Proteins were expressed in E. coli BL21(DE3) cells under the control of the IPTG inducible T7 promoter. 1 liter culture of BL21(DE3) cells expressing sfCherry, sfCherry with GFP strand 10-11 inserted were grown to OD^{600nm}˜0.5-0.7 and induced with 1 mM IPTG for 7 hours at 30° C. The harvested cells were suspended in TNG buffer and lysed by sonication on ice for 10 minutes at 70% duty cycle. The mixture then was centrifuged at 15,000×g for 30 minutes at 10° C. to remove cell debris. For sfCherry and sfCherry-GFP10-11, the supernatant was incubated with pre-equilibrated Talon® metal affinity resin (Clontech) and the mixture was incubated at room temperature with gentle shaking for 1 hour to allow proteins to bind to resin. The proteins bound to resin was separated from unbound ones by centrifuging 3,000×g for 5 minutes and the resin was washed two times with column buffer before it was packed into a gravity-flow column. The column then was washed with 50 ml of column buffer [50 mM Na phosphate buffer pH 7, 300 mM NaCl, 10% glycerol] followed by 50 ml of binding buffer [50 mM Na phosphate buffer pH 7, 300 mM NaCl, 10% glycerol, 5 mM imidazole] and 20 ml of washing buffer [50 mM Na phosphate buffer pH 7, 300 mM NaCl, 10% glycerol, 20 mM imidazole] to remove unbound and non-specifically bound proteins, respectively. The purified proteins were completely eluted with 250 mM imidazole in TNG buffer with a good yield: about 40 mg/l of cell culture. Protein solutions were concentrated and exchanged to final buffer [20 mM Tris-HCl pH8, 150 mM NaCl, 1 mM dithiothreitol (DTT)] using an Amicon Ultra-15 centrifugal filter device (10 kDa cutoff; Millipore).

To create sfCherry-GFP10-11/GFP1-9 protein complex, purified sfCherry-GFP10-11 was complemented overnight in the cold room with an excessive amount of refolded GFP1-9 such that the amount of GFP1-9 is not limiting. Protein mixture was applied to pre-equilibrated Talon® metal affinity resin and the protein complex was subsequently purified using the same purification protocol as for sfCherry and sfCherry-GFP10-11 indicated above. For each purification step, the protein elution samples were resolved on a 4-20% gradient CRITERION™ SDS-PAGE gel (Bio-Rad, Hercules, Calif.) and stained using Gel Code Blue stain reagent (Pierce, Rockford, Ill.).

In Vitro Complementation Assays of sfCherry-GFP10-11 (D169/G170) with GFP1-9 or GFP1-10.

In vitro complementation assays were done as previously described (Cabantous, et al., Nature Methods 3, 845-854, 2006). A 96-well microplate (Nunc-Immuno plate, Nunc) was first blocked with a solution of 0.5% bovine serum albumin (BSA) in TNG for 10 minutes. Purified sfCherry-GFP10-11 hairpin was subjected to twofold serial dilutions in the same buffer so that the dilutions spanned the range 1 to 200 pmol per 20 μl aliquot. Protein aliquots were added to 96-well plates and complementation was performed using a large excess of GFP1-9 or GFP1-10 (˜1 mg/ml, 800 pmol) added in a 180 μl aliquot. Fluorescence kinetics (488 nm excitation/520 nm emission) was monitored with a DTX™ Microplate Fluorescence Reader (Beckman Coulter) at 3 min intervals for 15 hours. The background fluorescence of a blank sample (20 μl of 0.5% BSA in TNG buffer, 180 μl of 1 mg/ml GFP1-9 or GFP1-10 in TNG buffer) was subtracted from the final fluorescence values.

Crystallization.

SfCherry (concentration ˜25 mg/ml) and sfCherry-GFP10-11 hairpin/GFP1-9 complex (concentration ˜22 mg/ml) were both crystallized using the sitting-drop vapor diffusion method by mixing 0.15 μl of protein stocks with 0.15 μl reservoir solution equilibrated against 30 μL reservoir at 298K. A set of 384 crystallization reagents consisting of Crystal Screens I, II (Hampton Research), PACT™ suite (Qiagen), and JCSG core suites I and II (Qiagen) was used to screen for the propensity of crystallization. Subsequent optimization fine-tuning pH, salt, precipitants, and additives were employed as needed until diffraction-quality crystals were obtained.

For sfCherry crystallization, six conditions from the initial screening, including four closely-related conditions from the A and B rows of the PACT suite appeared to be in the crystallization zone of sfCherry and yielded long, clustered needles or rods. The best crystals (˜200 μm×20 μm×10 μm) were obtained from the condition with 0.1M SPG buffer (succinic acid, phosphate, glycine) pH 5.0 and 25% (w/v) PEG 1500. Diffraction data from these crystal contained satellite lattices but one of the data sets was suitable for structure determination of sfCherry.

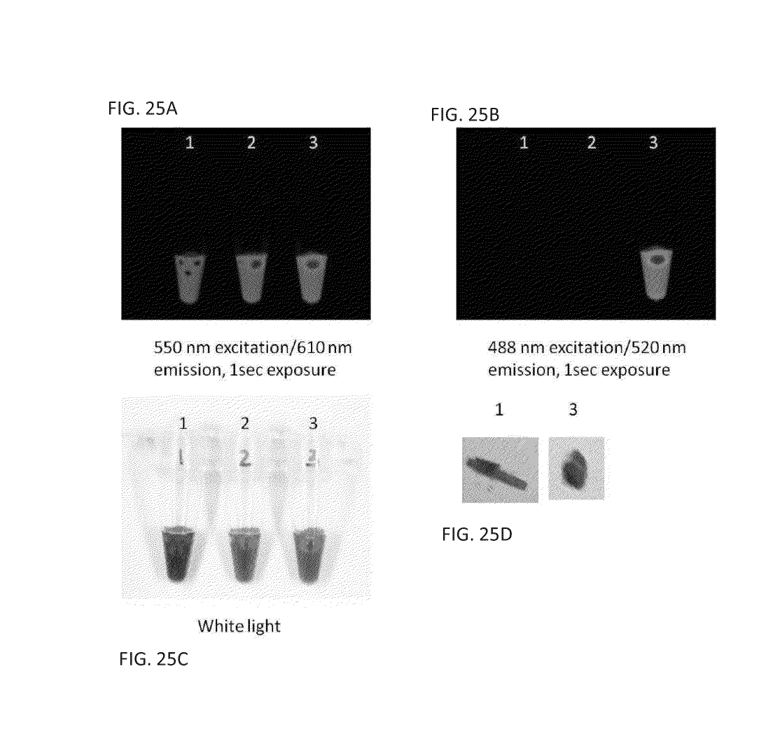

For sfCherry-GFP10-11 hairpin/GFP1-9 complex crystallization, clustered plates were observed in initial screening experiments in five conditions from rows E, F, and H of the PACT suite. Subsequent optimizations, including using glycerol as an additive to reduce nucleation yielded diffraction-quality crystals (100 μm×30 μm×20 μm) from a condition containing 0.1M Bis-Tris buffer pH 8.3, 20% (w/v) PEG 3350, and 6% (v/v) glycerol. Fluorescence microscopy was used to verify the existence of fluorophores in the crystals. Images of crystals taken under white light and photos of protein solutions taken with white light and under 488 nm/520 nm, 550 nm/610 nm excitation/emission filters are shown in FIG. 25.

Data Collection, Molecular Replacement and Refinement.

Crystals of sfCherry and sfCherry-GFP10-11 hairpin/GFP1-9 complex were collected at beam line 5.0.2 at the Advanced Light Source (ALS) and data were processed with the HKL2000 program (Otwinowski, et al., Methods enzymol., edited by C. W. J. Carter & R. M. Sweet, pp. 307-326. New York: Academic Press, 1997). The crystals of sfCherry belong to the P21 space group with cell dimensions of a=85.105 Å, b=96.294 Å, c=105.957 Å, β=104.56°. The data set was processed to 2.0 Å with an Rmerge of 9.5% and a completeness of 97.0%. Cell content analysis gave a Matthews coefficient of 2.17 Å³Da⁻¹ and a solvent content of 43% with eight copies of sfCherry in the asymmetric unit. The crystals of sfCherry-GFP10-11 hairpin/GFP1-9 complex belongs to the space group P212121 with cell dimensions of a=74.360 Å, b=86.490 Å, c=167.941 Å. The data set of sfCherry-GFP was processed at 2.6 Å with an Rmerge of 6.5% and a completeness of 98.8%. The Matthews coefficient of sfCherry-GFP10-11 hairpin/GFP1-9 complex crystals was 2.70 Å³Da⁻¹, suggesting a solvent content of 54% with two copies for the complex in the asymmetric unit.

The crystal structure of sfCherry was determined with the molecular replacement (MR) method using the PHASER program (McCoy, et al., J. Appl. Cryst. 40, 658-674, 2007) in the PHENIX suite (Adams, et al., Acta Cryst. D66, 213-221, 2010). The mCherry (2H5Q) (Shu, et al., Biochemistry 45, 9639-9647, 2006) structure was used as search model. Model rebuilding was carried out with the AutoBuild (Terwilliger, et al., Acta Cryst. D64, 61-69, 2008) program and refined with phenix.refine (Headd, et al., Acta Cryst. D68, 381-390, 2012; Afonine, et al., Acta Cryst. D68, 352-367, 2012). The final R- and free R-values for sfCherry were 22.2% and 26.5%, respectively.

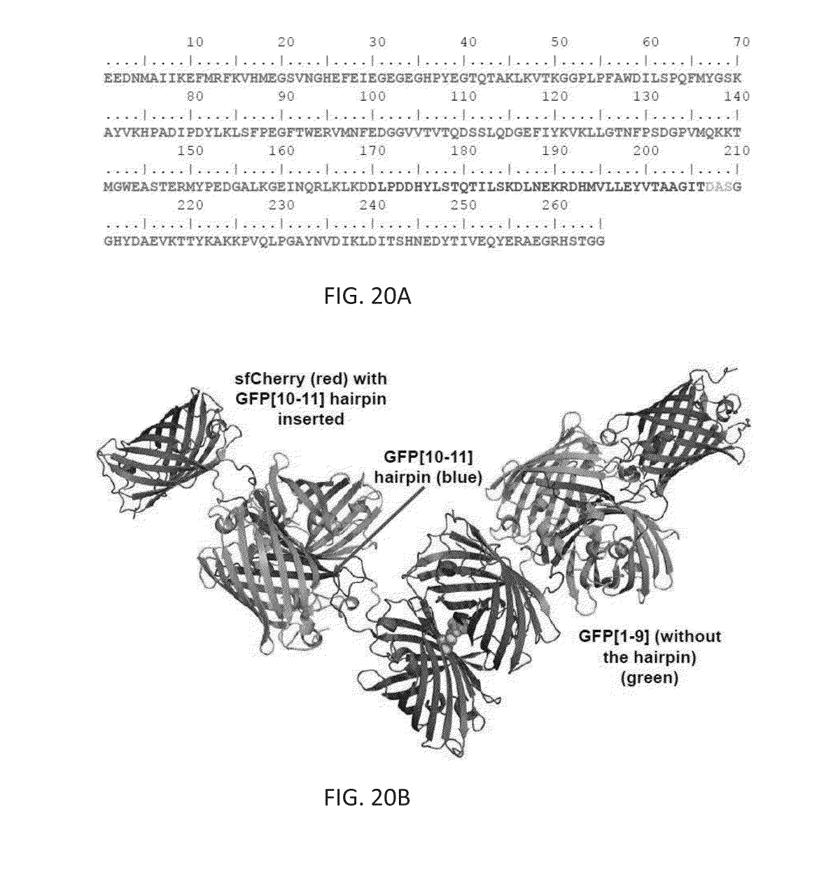

The crystal structure of sfCherry-GFP10-11 hairpin/GFP1-9 complex was also determined with the MR method. Similar procedures and programs as those used in the sfCherry structure determination were employed except for the following differences. The sfGFP (PDB code: 2B3Q) (Pedelacq, et al., Nature Biotechnol. 24, 79-88, 2006) and partially refined sfCherry structures were used as search models. The sequences belonging to strand 10 and 11 of the sfGFP were pruned from sfGFP and grafted between original strands 8 and 9 of sfCherry based on the designed constructs (FIG. 20A). This modified sequence pair was used in model rebuilding with AutoBuild. Reference-structure restraints (Headd, et al., Acta Cryst. D68, 381-390, 2012) were used in early stages of refinement and released at later stages. The refined structure of sfCherry-GFP10-11 hairpin/GFP1-9 complex has an R-value of 20.5% and a free R-value of 24.9%. Detailed data collection and refinement statistics of sfCherry and the sfCherry-GFP10-11 hairpin/GFP1-9 complex are listed in Table 2. The atomic coordinates and structure factors are available in the Protein Data Bank under accession code 4KF4 (sfCherry) and 4KF5 (sfCherry GFP10-11 hairpin/GFP1-9)

### Results

Strategy for Modular Design.

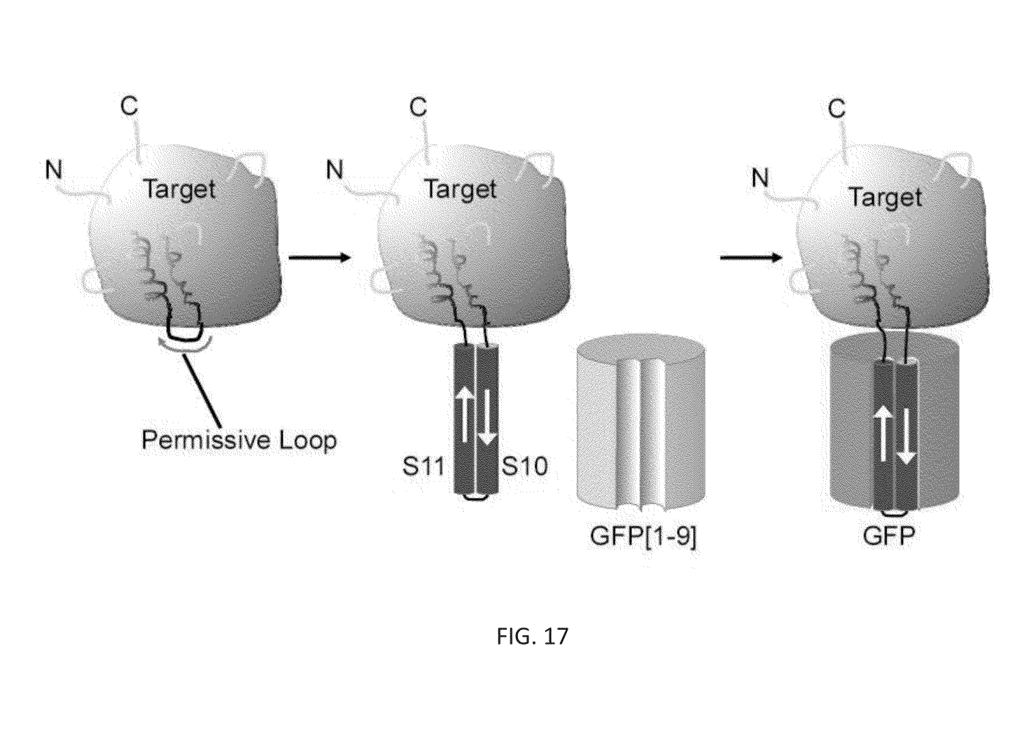

The structure, stability, and folding of GFP has been well studied (Ormo et al., Science, 273:1392-5, 1996; Tsien, Annu Rev Biochem 67, 509-544, 1998; Crameri, et al., Nature Biotechnol. 14, 315-319, 1996). Its relatively simple topology, combined with its utility as a fluorescent reporter when correctly folded (Waldo, et al., Nature Biotechnol. 17, 691-695, 1999; Pedelacq, et al., Nature Biotechnol. 24, 79-88, 2006), has made it an attractive system for reconstitution from separately expressed protein fragments (Cabantous, et al., Nature Biotechnol. 23, 102-107, 2004). Following such a strategy, by fusing terminal segments of GFP to a crystallization target, the resulting construct might be recombined with the remaining complementary fragment of GFP to create a new complex for crystallization. In the context of crystallization strategies, a challenge presented by typical fusion methods is the flexibility introduced at the site of connection between the two protein components; free torsion angles are present where the polypeptide backbone makes its (single) crossing from one natural protein fold to the other. The value of having the polypeptide chain cross twice instead of once between two connected proteins has been demonstrated in experiments where T4 lysozyme was inserted into a loop of GPCR membrane proteins, giving a construct that yielded well-ordered crystals (Rosenbaum, et al., Science 318, 1266-1273, 2007; Cherezov, et al., Science 318, 1258-1265, 2007). The split GFP system (GFP1-9+GFP10-11) allows for a similar advantage. If strands 10 and 11, which ostensibly form a natural hairpin, can be inserted as a long extension into a surface loop of a target protein, then reconstitution with complementary GFP1-9 should give a tight non-covalent complex with two chain crossings between natural protein folds (FIG. 17). In practice, rational choices for points of insertion of strands 10-11 into exposed loops might be based on homology models, where available, or on bioinformatic predictions of loops (Lambert, et al., Bioinformatics 18, 1250-1256, 2002; Dovidchenko, et al., J Bioinform Comput Biol 6, 1035-1047, 2008; Jones, J. Mol. Biol. 292, 195-202, 1999). Here, a target for crystallization whose structure was known was elected, in order to test the strategy of loop insertion and crystallization in a favorable case.

Cherry Fluorescent Protein as a Target Protein.

In the present study, the protein chosen as a target for crystallization was superfolder Cherry (sfCherry), a version of red fluorescent protein engineered in the laboratory. sfCherry was chosen as a test protein so that the folding of the target could be monitored by red fluorescence while the GFP reconstitution could be monitored by green fluorescence. The well-folding sfCherry protein was created from the fluorescent monomeric Cherry protein (mCherry), (Shaner, et al., Nature Biotechnol. 22, 1567-1572, 2004) by directed evolution of the mCherry carrying the poorly folding and aggregation-prone bullfrog red-cell H-subunit ferritin as a N terminal fusion, as previously described (Pedelacq, et al., Nature Biotechnol. 24, 79-88, 2006). Owing to the naturally poor folding properties of the ferritin, colonies expressing the initial ferritin-mCherry fusion at 37° C. showed only faint fluorescence (FIG. 25A). After three rounds of DNA shuffling, during which the selected brighter fluorescent clones expressed at 37° C., highly fluorescent ferritin-sfCherry protein fusions were obtained. Escherichia coli (E. coli) colonies and liquid culture of cells expressing ferritin-sfCherry fusions after three rounds of directed evolution were about 100-fold brighter than cells expressing ferritin-mCherry at 37° C. (FIG. 25A). The new folding-enhanced sfCherry contains six mutations: R36H, K92T, R125L, S147T, K162N, and N196D. A native polyacrylamide gel at ˜10 mg/ml protein concentration indicated that the protein is approximately 50% dimer and 50% monomer (FIG. 25B).

Selection of a Permissive Insertion Site in sfCherry.

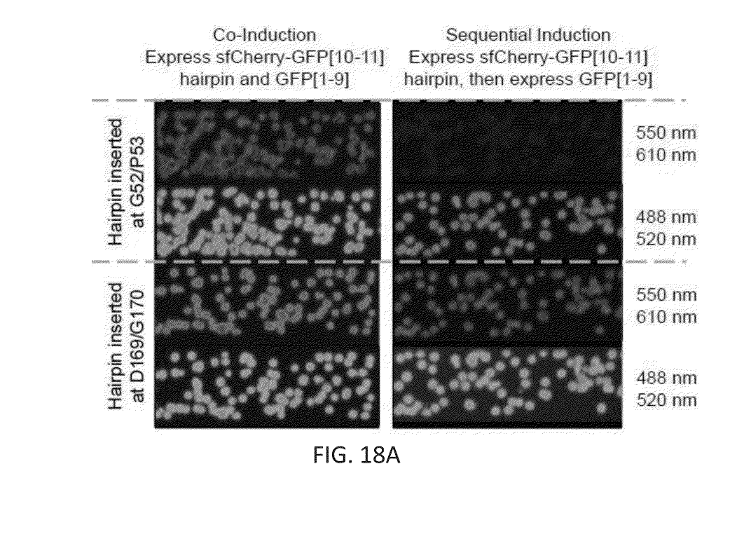

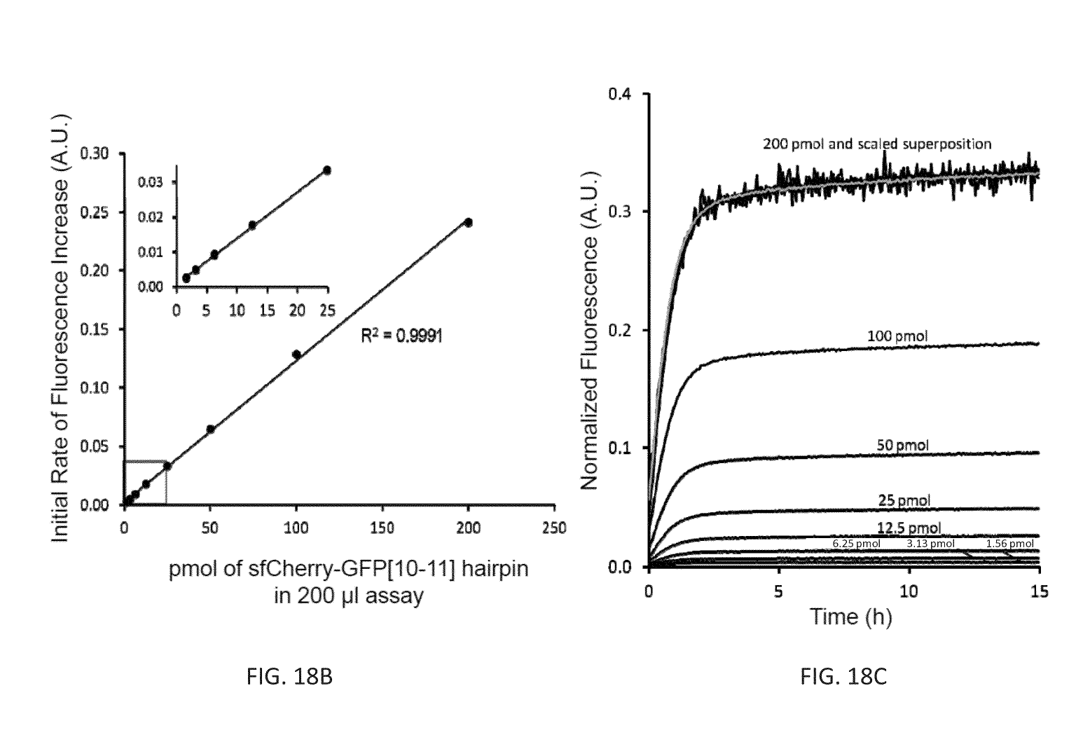

The strategy of inserting GFP strands 10-11 into a target protein requires that permissive sites be identified. In order to guide the choice of sites that might be permissive for insertion in the target protein, sfCherry, earlier experimental data for circular permutants of superfolder GFP (sfGFP) was partly relied on, which has 23.3% sequence identity to sfCherry and a similar structure (Pedelacq, et al., Nature Biotechnol. 24, 79-88, 2006). On that basis, the GFP10-11 hairpin with a short linker of 3 residues (DAS) was inserted at two different loop sites (G52/P53 or D169/G170) of sfCherry. The 3-residue linker was included to improve protein solubility as guided from previous experiments. These two sfCherry-GFP10-11 hairpin constructs were screened for expression and solubility in vivo in E. coli colonies using a complementation assay with GFP1-9 as previously described for GFP11 and GFP1-10 (Cabantous, et al., Nature Methods 3, 845-854, 2006). The construct with the GFP hairpin inserted at D169/G170 clearly showed brighter red and green fluorescence compared to the G52/P53 insertion (FIG. 18A). Insertion of the GFP hairpin at the permissive site D169/G170 of sfCherry was the better choice for folding of the target and subsequent binding to GFP1-9. This construct was chosen for further crystallization and structural characterization.

In-Vitro Complementation Assays of sfCherry-GFP10-11 Hairpin with GFP1-9 and GFP1-10.

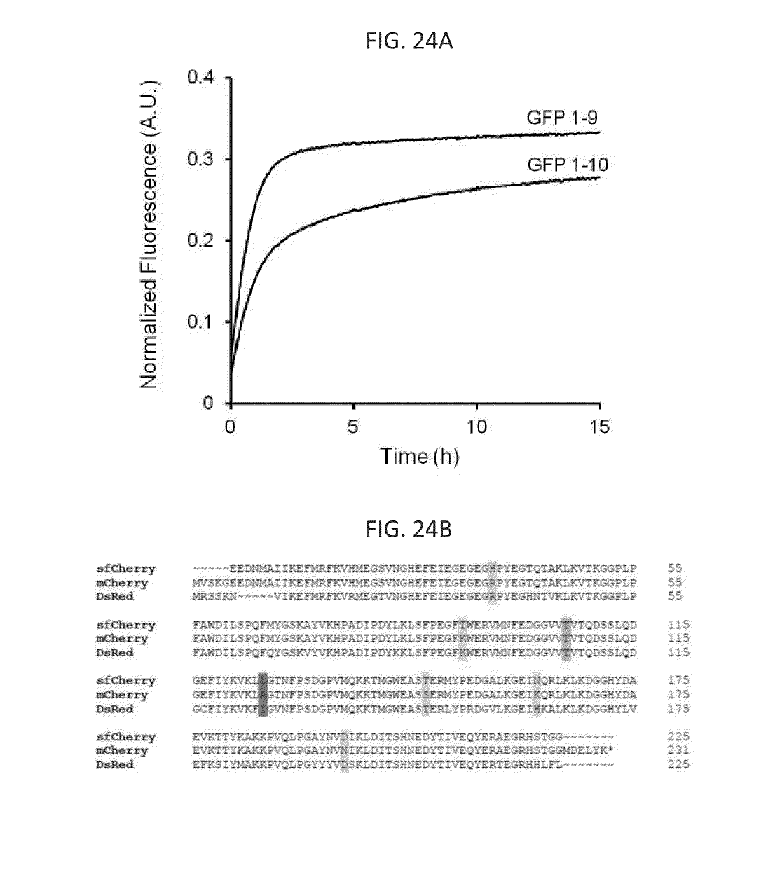

To characterize the sfCherry with the GFP10-11 hairpin inserted at D169/G170 in greater detail, in vitro different concentrations of sfCherry in the range of 1 to 200 pmol (in a 20 μl aliquot) were complemented by adding a large molar excess (800 pmol in a 180 μl aliquot) of either GFP1-9 or GFP1-10. As expected, the GFP10-11 hairpin inserted into sfCherry complemented the GFP1-9 molecule and yielded bright fluorescence (FIG. 24A). Additionally, GFP1-9 complemented the hairpin faster than did GFP1-10 (FIG. 24A). In the latter case, strand 10 in the reconstituted GFP could come either from the hairpin (requiring displacement of GFP strand 10 in the GFP1-10), or from the GFP1-10 (requiring displacement of GFP strand 10 in the 10-11 hairpin). Potential steric hindrance from two copies of GFP strand 10 might explain the reduced kinetics with GFP1-10, but was beyond the scope of the present work and was not explored further.

Initial complementation rates of sfCherry-GFP10-11 hairpin with GFP1-9 were a linear function of the concentration of sfCherry-GFP10-11 hairpin (FIG. 18B). Scaled complementation curves were superimposable, indicating a mechanism that is independent of the concentration of the sfCherry-GFP10-11 hairpin (FIG. 18C), as expected since the GFP1-9 was present in large excess.

Crystal Structure of sfCherry Alone.

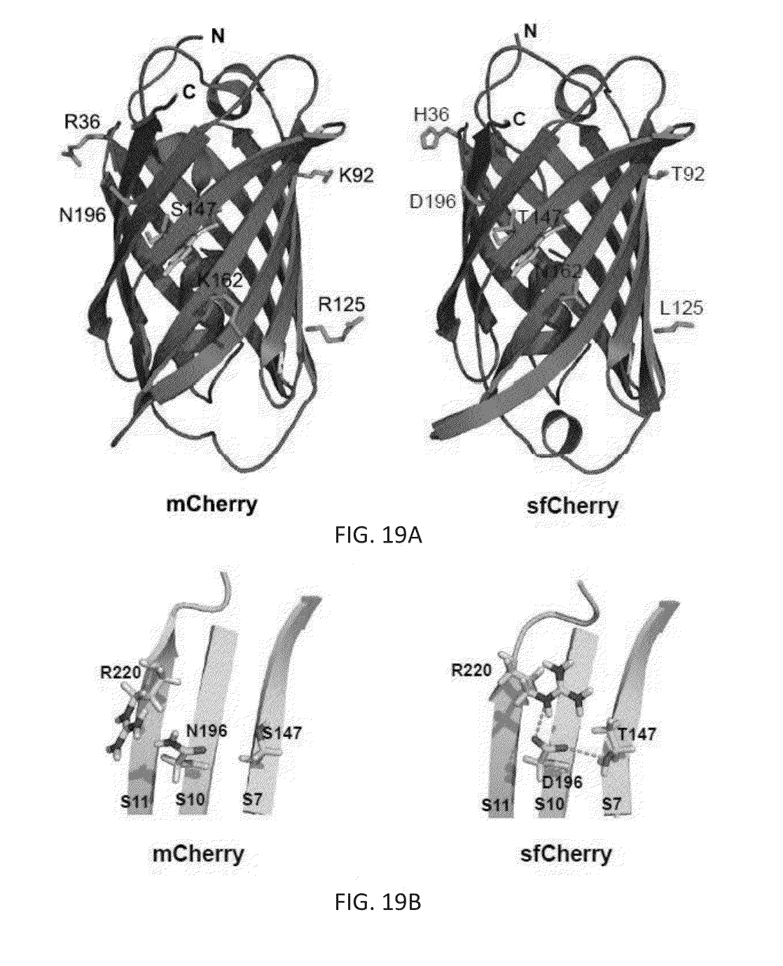

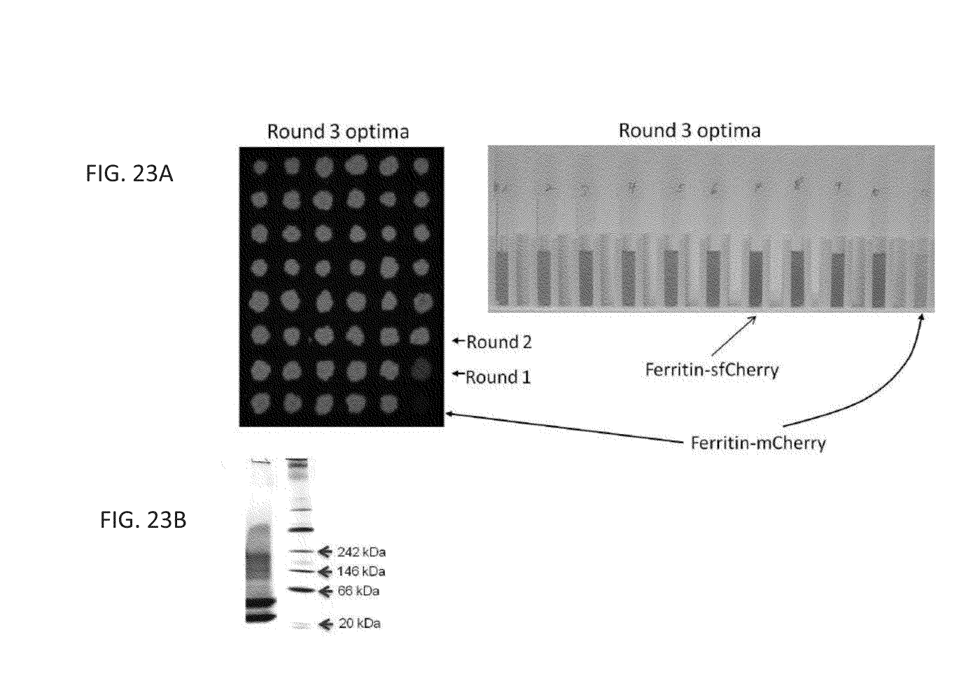

To allow subsequent comparisons, the crystal structure of sfCherry (without a loop insertion) was determined at 2 Å resolution from the protein expressed in E. coli (Table 2). The C_{α} superposition of sfCherry and mCherry, (PDB code 2H5Q (Shu, et al., Biochemistry 45, 9639-9647, 2006) has a root mean square deviation (rmsd) of only 0.17 Å for residues 6 to 223 (FIG. 19A). The chromophore is formed from residues M66-Y67-G68 and is buried in the middle of the central helix. Unlike mCherry, sfCherry crystallized as a symmetric dimer. The dimer interface includes hydrophobic residues V96, V104, L125 and hydrophilic residues N23, E94, T106, T108, T127, N128. Similar to the AB dimer interface found in Dsred tetramer (Yarbrough, et al., Proc. Natl. Acad. Sci. USA 98, 462-467, 2001), the sequence V104, T106, T108 is central to the dimer interface in the sfCherry structure, where T106A forms a hydrogen bond with its counterpart T106B. A sequence alignment of sfCherry, mCherry and Dsred (FIG. 24B) suggests that the R125L mutation in sfCherry likely contributes to the observed dimerization. In both the Dsred tetramer (PDB code: 1G7K) and the sfCherry structures (this work), either 1125 (Dsred) or L125 (sfCherry) may stabilize the dimer through hydrophobic interactions. In the mCherry structure (PDB code: 2H5Q), the bulky charged side chain R125 likely prevents dimerization by charge repulsion. In the sfCherry structure, the side chains of D196 form hydrogen bonds with R220 via O-δ2 and with T147 via O-δ1 while the corresponding interactions between N196 and R220/S147 are not present in the mCherry structure (FIG. 19B). This change in the hydrogen-bonding network, together with the R125L mutation, may explain in part why sfCherry is more stable and more tolerant to folding interference compared to mCherry when fused to a poorly folding and aggregation-prone protein such as H-subunit ferritin (FIG. 23A).

TABLE 2
Statistics of data collection and refinement for sfCherry (PDB entry 4KF4)
and the sfCherry GFP10-11 hairpin/GFP1-9 complex (PDB entry 4KF5)

| | | | sfCherry GFP10-11/ | |
|---|---|---|---|---|
| Data Collection | sfCherry | | GFP1-9 complex | |
| Wavelength (Å) | 1.0 Å | | 1.0 Å | |
| Resolution (Å) | 50.00-2.00 | (2.03-2.00)^{a} | 50.00-2.60 | (2.64-2.60)^{a} |
| Number of | 247,180 | | 142,199 | |
| observations | | | | |
| Number of unique | 109,059 | (5,459) | 63,036 | (2,956) |
| reflections | | | | |
| Completeness (%) | 97.0 | (98.1) | 98.8 | (94.0) |
| R_{merge} (%)^{b} | 9.5 | (54.6) | 6.5 | (47.7) |
| I/σ | 10.5 | (1.6) | 18.8 | (2.2) |
| Redundancy | 2.3 | (2.2) | 4.2 | (4.2) |
| Refinement | | | | |
| Resolution (Å) | 50.0-2.00 | (2.05-2.0) | 50.0-2.60 | (2.67-2.60) |
| ^{c}R_{cryst} (%) | 22.24 | (27.74) | 20.54 | (32.81) |
| ^{c}R_{free} (%) | 26.48 | (32.42) | 24.89 | (38.00) |

| Rmsd bonds (Å) | 0.008 | 0.005 |
|---|---|---|
| Rmsd angles (°) | 1.059 | 0.895 |
| Average B value | 27.9 | 83.2^{d} |
| protein (Å²) | | |
| Average B value | 29.2 | 59.4 |
| water (Å²) | | |
| Ramachandran Plot | (%) | (%) |
| most favored | 98.1 | 96.6 |
| Allowed | 1.9 | 3.4 |
| Outliers | 0 | 0 |

^{a}Values in parenthesis refer to the highest resolution shell.
^{b}R_{merge} = [Σₕ Σᵢ|Iᵢ(h) − <I(h)>|/Σₕ I(h)] × 100, where Iᵢ(h) is the i^{th} measurement of the h reflection and <I(h)> is the average value of the reflection intensity
^{c}R_{cryst/}R_{free} = (Σₕₖₗ|Fₒ − F_{c}|/Σₕₖₗ|Fₒ|) × 100
^{d}Average B values by protein chain (Å²): A = 96.9, B = 98.9, C = 71.6, D = 73.2

Structure of sfCherry with GFP Strands 10-11 Inserted at D169/G170 in a Complex with GFP1-9.

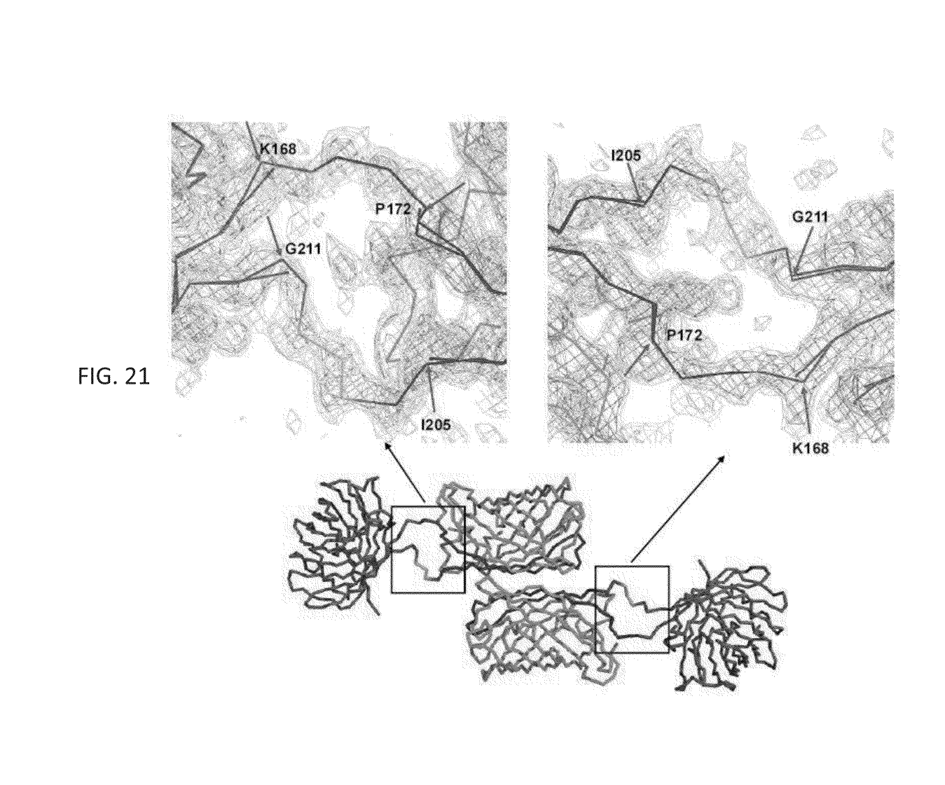

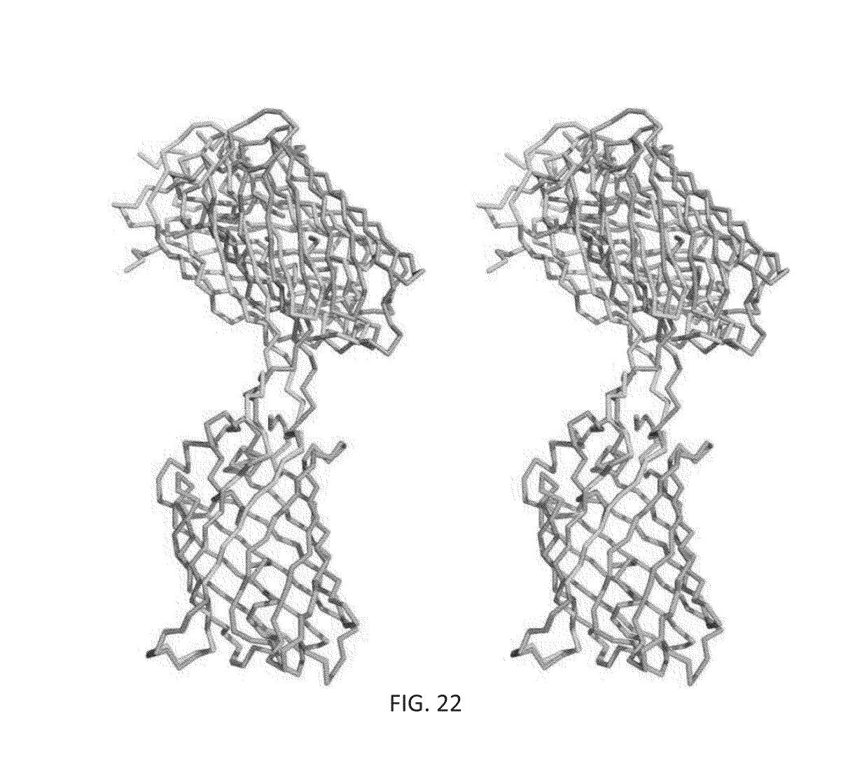

The structure of the sfCherry-GFP10-11 in complex with GFP1-9 was determined at 2.6 Å resolution, with a final R/Rfree of 0.205/0.247 (Table 2). No major elements of disorder, conformational heterogeneity, or anisotropy were observed. The structure of the complex (FIG. 20B) shows sfCherry clearly fused to the GFP10-11 hairpin, and with the GFP10-11 hairpin complementing GFP1-9 to form the intact GFP molecule. The crystal asymmetric unit contains two copies of the complex. With two complexes in the asymmetric unit, and two linking chain segments between the two protein components in each case, there are four linking polypeptide segments. All of these segments are well-ordered and clearly visible in the final electron density map (FIG. 21). Furthermore, the relative orientation of the GFP and sfCherry components in the complex is very similar in the two instances visualized in the asymmetric unit. When the GFP components of the two independent complexes are spatially overlapped, the sfCherry components differ in the two cases by a rotation of only 9 degrees (FIG. 22).

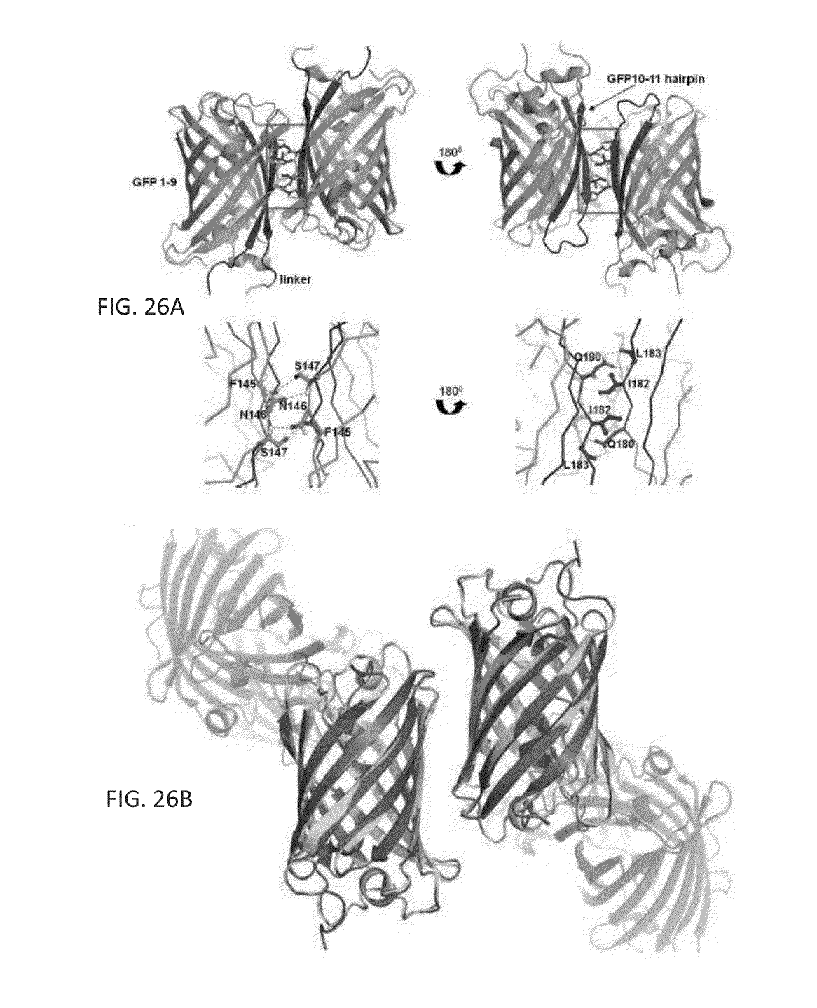

The GFP domains form a dimer in the crystal with local two-fold symmetry (FIG. 20B and FIG. 26A). The GFP dimer interface is mediated through β strand 10 (inserted in sfCherry) via the sequence Q180, I182, L183 and the loop F145, N146, S147 connecting strand 6 and strand 7 of GFP1-9. Residue Q180 of GFP β strand 10 is hydrogen bonded to the backbone of its counterpart L183 via N-ε and O-ε. Position 1182 in the sfCherry-GFP10-11 hairpin construct corresponds to A206 in the folding reporter GFP and to V206 in sfGFP (Pedelacq, et al., Nature Biotechnol. 24, 79-88, 2006). The dimer interface found in the folding reporter GFP crystal structure (PDB code: 2B3Q) was also mediated through Q204, A206, L207 of strand 10 and Y145, N146 and S147 of the loop connecting strand 6 and strand 7 (Pedelacq, et al., Nature Biotechnol. 24, 79-88, 2006) similar to the interface found in the sfCherry-GFP10-11 hairpin/GFP1-9 complex structure. The sfCherry domains are arranged in the crystal as a dimer that is essentially identical to the dimer formed when crystallized by itself (FIG. 20B and FIG. 26B). The crystal structure exhibits strong packing interactions in all three dimensions due to the dimerization of the reconstituted GFP, the linkage between GFP and sfCherry (creating linkages in the x-y plane), and the dimerization of sfCherry (creating linkages in the z-direction).

### Discussion

The purpose of these experiments was to develop a modular framework for using split GFP as a crystallization partner. A proof of principle experiment in which GFP reconstitution was used to monitor the success of the GFP hairpin insertion into sfCherry, a red fluorescent protein is presented, and the atomic structure of the sfCherry-GFP10-11 hairpin/GFP1-9 protein complex is determined by X-ray diffraction. The GFP10-11 hairpin described here was originally optimized as a protein interaction detector with each beta strand separately attached to an interacting protein. Part of that optimization involved eliminating any aggregation and self-assembly between the beta strands. This could potentially destabilize the GFP10-11 hairpin prior to complementation by GFP1-9, affecting the stability of target proteins. Despite these caveats, a site for insertion of the GFP10-11 hairpin sequence was identified that did not substantially disrupt the folding of the well-folded sfCherry. However, the insertion of the GFP10-11 hairpin might affect the stability of less stable target proteins. The choice of insertion site might therefore be important in more general applications. For choosing the permissive sites of sfCherry in this study, homology models were partly relied on (below) as well as earlier experimental data for circular permutants of sfGFP. A homology model obtained for sfCherry sequence using SWISS-MODEL (Arnold, et al., Bioinformatics 22, 195-201, 2006; Guex, et al., Electrophoresis 18, 2714-2723, 1997; Schwede, et al., Nucleic Acids Res. 31, 3381-3385, 2003) has an RMSD of 0.2 Å for C_{α} atoms of residues 6 to 222 compared to the actual structure that was obtained for sfCherry in this study. The GFP10-11 hairpin sequence could have been inserted into any of several loop sites of sfCherry based on this homology model; in vivo experiments (section 2.2) could have been used to screen for the most permissive site. The GFP10-11 hairpin sequence reported in this paper is likely to be suitable for insertion into various other target proteins.

While some structures have previously been obtained for proteins fused terminally to full length GFP, the use instead of the GFP hairpin insertion as a fusion partner has potential benefits for crystallization. The hairpin is small and may be less perturbing of protein folding than fusion of intact GFP. Further, the hairpin is topologically well suited for insertion into loops and turns of a target protein. Finally, instead of a single chain crossing afforded by terminal GFP fusions, the hairpin provides two chain crossings between the target and the reconstituted GFP. This likely is an important feature, as it would be expected to reduce the flexibility between the connected components. This expectation was confirmed by the crystal structure of the complex. It was observed that the chain crossing segments were well ordered. Perhaps more compelling, the two instances of the complex seen in the asymmetric unit of the crystal suggest that the two connected components, GFP and the sfCherry target protein, sample a rather limited range of relative orientations. The relative orientation of the two components differs by 9 degrees when the two complexes are compared. This appears essentially as a minor hinge motion through the two points of connection; twisting and rotation about the other orthogonal direction are evidently limited by the double connection. The connection therefore appears to be relatively rigid.

The ease with which the current version of GFP strands 10-11 could be inserted into a test protein, and then readily crystallized as a complex with GFP1-9, suggests that the approach may be widely applicable, especially after further optimization of the GFP10-11 hairpin. The case presented here held the advantage that the target protein had already been structurally characterized, so that surface loops for insertion could be defined easily. For more realistic applications, homology modeling could be valuable in selecting prospective insertion sites. In the most challenging cases, such as where the target protein has no homologs of known structure, a library of constructs with the hairpin randomly inserted could be created, and the in vivo solubility assay with GFP1-9 (described in this study) could be used to screen for permissive sites.

The natural modularity of the split system for crystallization opens the possibility of engineering and testing many variants of GFP1-9 that might be expected to have distinct crystallization behaviors. In this way, a single target protein construct bearing a GFP10-11 insertion could be combined with any number of different variants of the GFP1-9 carrier, leading to greatly expanded chances for crystallization. This strategy would circumvent the labor associated with exhaustively re-engineering a protein being targeted for crystallization, since the purified target protein bearing the GFP hairpin could be complemented with different pre-purified GFP1-9 mutants without further genetic manipulation, protein expression and purification. The strategy shown here could apply to detergent-solubilized membrane proteins as well, inserting the GFP10-11 hairpin into exposed cytoplasmic loops, as for soluble proteins. Another benefit of this system is that GFP can potentially be used as the search model in molecular replacement, making it possible to obtain diffraction phases and electron density maps even for a target protein with an unknown fold.

Many of the techniques that have been used to vary the crystallization behavior of proteins could be employed to modify the GFP1-9 carrier. In particular, synthetically symmetrized versions of GFP1-9 should lead to highly distinct constructs, with each providing essentially independent opportunities for forming lattice contacts during crystallization.

TABLE 3
List of the primers used to insert GFP10-11 hairpin into G52/P53
and D169/G170 permissive loops of sfCherry

| | | SEQ |
|---|---|---|
| Primer name | Primer sequence 5′→3′ | ID NO |
| G52/P53 left top | GCAAGGAATGGTGCATGCAAGGAGATGGCGCCCAACAG | 27 |
| G52/P53 left bottom 1 | GACCATGTGGTCACGCTTTTCGTTGAGATCTTTCGAAAG | 28 |
| G52/P53 left bottom 2 | CTTTTCGTTGAGATCTTTCGAAAGGATAGTTTGTGTCGAC | 29 |
| G52/P53 left bottom 3 | TTCGAAAGGATAGTTTGTGTCGACAGGTAATGGTCGTC | 30 |
| G52/P53 left bottom 4 | TGTGTCGACAGGTAATGGTCGTCTGGTAAATCTCCTCCT | 31 |
| G52/P53 left bottom 5 | GTCGTCTGGTAAATCTCCTCCTTTAGTGACTTTCAATTTAG | 32 |
| G52/P53 right top 1 | TCGAAAGATCTCAACGAAAAGCGTGACCACATGGTCCT | 33 |
| G52/P53 right top 2 | CGAAAAGCGTGACCACATGGTCCTTCTTGAGTATGTAAC | 34 |
| G52/P53 right top 3 | TGGTCCTTCTTGAGTATGTAACTGCTGCTGGGATTACAG | 35 |
| G52/P53 right top 4 | GTAACTGCTGCTGGGATTACAGATGCATCTCCTCTTCCA | 36 |
| G52/P53 right top 5 | ATTACAGATGCATCTCCTCTTCCATTCGCCTGGGATATAC | 37 |
| G52/P53 right bottom | GAGGCCTCTAGAGGTTATGCTAGTTATTGC | 38 |
| D169/G170 left top | GCAAGGAATGGTGCATGCAAGGAGATGGCGCCCAACAG | 39 |
| D169/G170 left bottom 1 | GACCATGTGGTCACGCTTTTCGTTGAGATCTTTCGAAAG | 40 |
| D169/G170 left bottom 2 | CTTTTCGTTGAGATCTTTCGAAAGGATAGTTTGTGTCGAC | 41 |
| D169/G170 left bottom 3 | TTCGAAAGGATAGTTTGTGTCGACAGGTAATGGTCGTC | 42 |
| D169/G170 left bottom 4 | TGTGTCGACAGGTAATGGTCGTCTGGTAAATCATCTTTAAG | 43 |
| D169/G170 left bottom 5 | GTCGTCTGGTAAATCATCTTTAAGCTTGAGGCGTTGATTG | 44 |
| D169/G170 right top 1 | TCGAAAGATCTCAACGAAAAGCGTGACCACATGGTCCT | 45 |
| D169/G170 right top 2 | CGAAAAGCGTGACCACATGGTCCTTCTTGAGTATGTAAC | 46 |
| D169/G170 right top 3 | TGGTCCTTCTTGAGTATGTAACTGCTGCTGGGATTACAG | 47 |
| D169/G170 right top 4 | GTAACTGCTGCTGGGATTACAGATGCATCTGGTGGCCAT | 48 |
| D169/G170 right top 5 | ATTACAGATGCATCTGGTGGCCATTACGATGCAGAGGTTA | 49 |
| D169/G170 right bottom | GAGGCCTCTAGAGGTTATGCTAGTTATTGC | 50 |

### Example 3

### Circular Permutant SFP detectors

This example describes circular permutant SFP detectors based on the SFP detectors described in Example 1. The SFP detectors described below include nine contiguous β-strands of GFP or a circular permutant thereof, wherein the GFP includes the amino acid substitutions described in Example 1 for the GFP1-9 WT, OPT1, or OPT2 detectors. The SFP detectors can be complemented with the remaining two β-strands of the GFP to form a fluorescent protein.

FIG. 27 illustrates an example of circular permutant starting at breakpoint indicated by circled 11. The new amino acid start is 173. Translation proceeds through amino acid 233, then through the linker connecting strand 11 to β-strand 1. However, the N- and C-terminus of the circular permutant can be placed at any of the regions between β-strands on the GFP β-barrel. A linker (e.g., a glycine-serine linker) is placed between the native N- and C-termini.

The nucleic acid and protein sequences of exemplary SFP detectors including nine contiguous β-strands of GFP based on the GFP1-9 OPT1 detector described in Example 1 are provided below. With reference to the circled number in FIG. 27, the sequence names are (1) GFP2-10, (2) GFP3-11, (3) GFP4-1a, (4) GFP4-1b, (5) GFP5-2, (6) GFP6-3, (7) GFP7-4a, (8) GFP7-4b, (9) GFP7-4c, (10) GFP8-5, (11) GFP9-6, (12) GFP10-7a, (13) GFP10-7b, and (14) GFP11-8. The first amino acid with reference to the GFP sequence is also provided in the nucleic acid sequence and corresponding amino acid sequences for the SFP detectors listed below:

(1) GFP2-10, starting at GFP AA 23
(SEQ ID NO: 51)
ATGGGGCACAAATTTTTTGTCCGTGGAGAGGGTGAAGGTGATGCTACAATCGGAAAACTCAGC
CTTAAATTTATTTGCACTACTGGAAAACTACCTGTTCCATGGCCAACACTTGTCACTACTCTGAC
CTATGGTGTTCAATGCTTTTCCCGTTATCCGGATCACATGAAACGGCATGACTTTTTCAAGAGTG
CCATGCCCGAAGGTTATGTACAGGAACGCACTATATATTTCAAAGATGACGGGACCTACAAGA
CGCGTGCTGAAGTCAAGTTTGAAGGTGATACCCTTGTTAATCGTATCGAGTTAAAGGGTATTGA
TTTTAAAGAAGATGGAAACATTCTTGGACACAAACTCGAGTACAACTTTAACTCACACAAAGTA
TACATCACGGCAGACAAACAAAATAATGGAATCAAAGCTAACTTCACAATTCGCCACAACGTT
GAAGATGGTTCCGTTCAACTAGCAGACCATTATCAACAAAATACTCCAATTGGCGATGGCCCTG
TCCTTTTACCAGACGACCATTACCTGTCGACACAAACTATCCTTTCGAAAGATCTCAACGAAAA
GCGTGACCACATGGTCCTTCTTGAGTATGTAACTGCTGCTGGGATTACAGATGCTAGCGGTGGA
GGGTCTGGTGGCGGATCAAGGAAAGGAGAAGAACTTTTCACTGGAGTCGTCCCAATTCTTATTG
AATTAGATGGTGATGTTAAT
(2) GFP3-11, starting at GFP AA 39
(SEQ ID NO: 52)
ATGGGAAAACTCAGCCTTAAATTTATTTGCACTACTGGAAAACTACCTGTTCCATGGCCAACAC
TTGTCACTACTCTGACCTATGGTGTTCAATGCTTTTCCCGTTATCCGGATCACATGAAACGGCAT
GACTTTTTCAAGAGTGCCATGCCCGAAGGTTATGTACAGGAACGCACTATATATTTCAAAGATG
ACGGGACCTACAAGACGCGTGCTGAAGTCAAGTTTGAAGGTGATACCCTTGTTAATCGTATCGA
GTTAAAGGGTATTGATTTTAAAGAAGATGGAAACATTCTTGGACACAAACTCGAGTACAACTTT
AACTCACACAAAGTATACATCACGGCAGACAAACAAAATAATGGAATCAAAGCTAACTTCACA
ATTCGCCACAACGTTGAAGATGGTTCCGTTCAACTAGCAGACCATTATCAACAAAATACTCCAA
TTGGCGATGGCCCTGTCCTTTTACCAGACGACCATTACCTGTCGACACAAACTATCCTTTCGAAA
GATCTCAACGAAAAGCGTGACCACATGGTCCTTCTTGAGTATGTAACTGCTGCTGGGATTACAG
ATGCTAGCGGTGGAGGGTCTGGTGGCGGATCAAGGAAAGGAGAAGAACTTTTCACTGGAGTCG
TCCCAATTCTTATTGAATTAGATGGTGATGTTAATGGGCACAAATTTTTTGTCCGTGGAGAGGGT
GAAGGTGATGCTACAATC
(3) GFP4-1a, starting at GFP AA 51
(SEQ ID NO: 53)
ATGAAACTACCTGTTCCATGGCCAACACTTGTCACTACTCTGACCTATGGTGTTCAATGCTTTTC
CCGTTATCCGGATCACATGAAACGGCATGACTTTTTCAAGAGTGCCATGCCCGAAGGTTATGTA
CAGGAACGCACTATATATTTCAAAGATGACGGGACCTACAAGACGCGTGCTGAAGTCAAGTTT
GAAGGTGATACCCTTGTTAATCGTATCGAGTTAAAGGGTATTGATTTTAAAGAAGATGGAAACA
TTCTTGGACACAAACTCGAGTACAACTTTAACTCACACAAAGTATACATCACGGCAGACAAACA
AAATAATGGAATCAAAGCTAACTTCACAATTCGCCACAACGTTGAAGATGGTTCCGTTCAACTA
GCAGACCATTATCAACAAAATACTCCAATTGGCGATGGCCCTGTCCTTTTACCAGACGACCATT
ACCTGTCGACACAAACTATCCTTTCGAAAGATCTCAACGAAAAGCGTGACCACATGGTCCTTCT
TGAGTATGTAACTGCTGCTGGGATTACAGATGCTAGCGGTGGAGGGTCTGGTGGCGGATCAAG
GAAAGGAGAAGAACTTTTCACTGGAGTCGTCCCAATTCTTATTGAATTAGATGGTGATGTTAAT
GGGCACAAATTTTTTGTCCGTGGAGAGGGTGAAGGTGATGCTACAATCGGAAAACTCAGCCTTA
AATTTATTTGCACTACTGGA
(4) GFP4-1b, starting at GFP AA 91
(SEQ ID NO: 54)
ATGTATGTACAGGAACGCACTATATATTTCAAAGATGACGGGACCTACAAGACGCGTGCTGAA
GTCAAGTTTGAAGGTGATACCCTTGTTAATCGTATCGAGTTAAAGGGTATTGATTTTAAAGAAG
ATGGAAACATTCTTGGACACAAACTCGAGTACAACTTTAACTCACACAAAGTATACATCACGGC
AGACAAACAAAATAATGGAATCAAAGCTAACTTCACAATTCGCCACAACGTTGAAGATGGTTC
CGTTCAACTAGCAGACCATTATCAACAAAATACTCCAATTGGCGATGGCCCTGTCCTTTTACCA
GACGACCATTACCTGTCGACACAAACTATCCTTTCGAAAGATCTCAACGAAAAGCGTGACCACA
TGGTCCTTCTTGAGTATGTAACTGCTGCTGGGATTACAGATGCTAGCGGTGGAGGGTCTGGTGG
CGGATCAAGGAAAGGAGAAGAACTTTTCACTGGAGTCGTCCCAATTCTTATTGAATTAGATGGT
GATGTTAATGGGCACAAATTTTTTGTCCGTGGAGAGGGTGAAGGTGATGCTACAATCGGAAAA
CTCAGCCTTAAATTTATTTGCACTACTGGAAAACTACCTGTTCCATGGCCAACACTTGTCACTAC
TCTGACCTATGGTGTTCAATGCTTTTCCCGTTATCCGGATCACATGAAACGGCATGACTTTTTCA
AGAGTGCCATGCCCGAAGGT
(5) GFP5-2, starting at GFP AA 102
(SEQ ID NO: 55)
ATGGACGGGACCTACAAGACGCGTGCTGAAGTCAAGTTTGAAGGTGATACCCTTGTTAATCGTA
TCGAGTTAAAGGGTATTGATTTTAAAGAAGATGGAAACATTCTTGGACACAAACTCGAGTACA
ACTTTAACTCACACAAAGTATACATCACGGCAGACAAACAAAATAATGGAATCAAAGCTAACT
TCACAATTCGCCACAACGTTGAAGATGGTTCCGTTCAACTAGCAGACCATTATCAACAAAATAC
TCCAATTGGCGATGGCCCTGTCCTTTTACCAGACGACCATTACCTGTCGACACAAACTATCCTTT
CGAAAGATCTCAACGAAAAGCGTGACCACATGGTCCTTCTTGAGTATGTAACTGCTGCTGGGAT
TACAGATGCTAGCGGTGGAGGGTCTGGTGGCGGATCAAGGAAAGGAGAAGAACTTTTCACTGG
AGTCGTCCCAATTCTTATTGAATTAGATGGTGATGTTAATGGGCACAAATTTTTTGTCCGTGGAG
AGGGTGAAGGTGATGCTACAATCGGAAAACTCAGCCTTAAATTTATTTGCACTACTGGAAAACT
ACCTGTTCCATGGCCAACACTTGTCACTACTCTGACCTATGGTGTTCAATGCTTTTCCCGTTATC
CGGATCACATGAAACGGCATGACTTTTTCAAGAGTGCCATGCCCGAAGGTTATGTACAGGAAC
GCACTATATATTTCAAAGAT
(6) GFP6-3, starting at GFP AA 117
(SEQ ID NO: 56)
ATGACCCTTGTTAATCGTATCGAGTTAAAGGGTATTGATTTTAAAGAAGATGGAAACATTCTTG
GACACAAACTCGAGTACAACTTTAACTCACACAAAGTATACATCACGGCAGACAAACAAAATA
ATGGAATCAAAGCTAACTTCACAATTCGCCACAACGTTGAAGATGGTTCCGTTCAACTAGCAGA
CCATTATCAACAAAATACTCCAATTGGCGATGGCCCTGTCCTTTTACCAGACGACCATTACCTGT
CGACACAAACTATCCTTTCGAAAGATCTCAACGAAAAGCGTGACCACATGGTCCTTCTTGAGTA
TGTAACTGCTGCTGGGATTACAGATGCTAGCGGTGGAGGGTCTGGTGGCGGATCAAGGAAAGG
AGAAGAACTTTTCACTGGAGTCGTCCCAATTCTTATTGAATTAGATGGTGATGTTAATGGGCAC
AAATTTTTTGTCCGTGGAGAGGGTGAAGGTGATGCTACAATCGGAAAACTCAGCCTTAAATTTA
TTTGCACTACTGGAAAACTACCTGTTCCATGGCCAACACTTGTCACTACTCTGACCTATGGTGTT
CAATGCTTTTCCCGTTATCCGGATCACATGAAACGGCATGACTTTTTCAAGAGTGCCATGCCCG
AAGGTTATGTACAGGAACGCACTATATATTTCAAAGATGACGGGACCTACAAGACGCGTGCTG
AAGTCAAGTTTGAAGGTGAT
(7) GFP7-4a, starting at GFP AA 129
(SEQ ID NO: 57)
ATGTTTAAAGAAGATGGAAACATTCTTGGACACAAACTCGAGTACAACTTTAACTCACACAAA
GTATACATCACGGCAGACAAACAAAATAATGGAATCAAAGCTAACTTCACAATTCGCCACAAC
GTTGAAGATGGTTCCGTTCAACTAGCAGACCATTATCAACAAAATACTCCAATTGGCGATGGCC
CTGTCCTTTTACCAGACGACCATTACCTGTCGACACAAACTATCCTTTCGAAAGATCTCAACGA
AAAGCGTGACCACATGGTCCTTCTTGAGTATGTAACTGCTGCTGGGATTACAGATGCTAGCGGT
GGAGGGTCTGGTGGCGGATCAAGGAAAGGAGAAGAACTTTTCACTGGAGTCGTCCCAATTCTT
ATTGAATTAGATGGTGATGTTAATGGGCACAAATTTTTTGTCCGTGGAGAGGGTGAAGGTGATG
CTACAATCGGAAAACTCAGCCTTAAATTTATTTGCACTACTGGAAAACTACCTGTTCCATGGCC
AACACTTGTCACTACTCTGACCTATGGTGTTCAATGCTTTTCCCGTTATCCGGATCACATGAAAC
GGCATGACTTTTTCAAGAGTGCCATGCCCGAAGGTTATGTACAGGAACGCACTATATATTTCAA
AGATGACGGGACCTACAAGACGCGTGCTGAAGTCAAGTTTGAAGGTGATACCCTTGTTAATCGT
ATCGAGTTAAAGGGTATTGAT
(8) GFP7-4b, starting at GFP AA 140
(SEQ ID NO: 58)
ATGCTCGAGTACAACTTTAACTCACACAAAGTATACATCACGGCAGACAAACAAAATAATGGA
ATCAAAGCTAACTTCACAATTCGCCACAACGTTGAAGATGGTTCCGTTCAACTAGCAGACCATT
ATCAACAAAATACTCCAATTGGCGATGGCCCTGTCCTTTTACCAGACGACCATTACCTGTCGAC
ACAAACTATCCTTTCGAAAGATCTCAACGAAAAGCGTGACCACATGGTCCTTCTTGAGTATGTA
ACTGCTGCTGGGATTACAGATGCTAGCGGTGGAGGGTCTGGTGGCGGATCAAGGAAAGGAGAA
GAACTTTTCACTGGAGTCGTCCCAATTCTTATTGAATTAGATGGTGATGTTAATGGGCACAAATT
TTTTGTCCGTGGAGAGGGTGAAGGTGATGCTACAATCGGAAAACTCAGCCTTAAATTTATTTGC
ACTACTGGAAAACTACCTGTTCCATGGCCAACACTTGTCACTACTCTGACCTATGGTGTTCAATG
CTTTTCCCGTTATCCGGATCACATGAAACGGCATGACTTTTTCAAGAGTGCCATGCCCGAAGGT
TATGTACAGGAACGCACTATATATTTCAAAGATGACGGGACCTACAAGACGCGTGCTGAAGTC
AAGTTTGAAGGTGATACCCTTGTTAATCGTATCGAGTTAAAGGGTATTGATTTTAAAGAAGATG
GAAACATTCTTGGACACAAA
(9) GFP7-4c, starting at GFP AA 145
(SEQ ID NO: 59)
ATGAACTCACACAAAGTATACATCACGGCAGACAAACAAAATAATGGAATCAAAGCTAACTTC
ACAATTCGCCACAACGTTGAAGATGGTTCCGTTCAACTAGCAGACCATTATCAACAAAATACTC
CAATTGGCGATGGCCCTGTCCTTTTACCAGACGACCATTACCTGTCGACACAAACTATCCTTTCG
AAAGATCTCAACGAAAAGCGTGACCACATGGTCCTTCTTGAGTATGTAACTGCTGCTGGGATTA
CAGATGCTAGCGGTGGAGGGTCTGGTGGCGGATCAAGGAAAGGAGAAGAACTTTTCACTGGAG
TCGTCCCAATTCTTATTGAATTAGATGGTGATGTTAATGGGCACAAATTTTTTGTCCGTGGAGAG
GGTGAAGGTGATGCTACAATCGGAAAACTCAGCCTTAAATTTATTTGCACTACTGGAAAACTAC
CTGTTCCATGGCCAACACTTGTCACTACTCTGACCTATGGTGTTCAATGCTTTTCCCGTTATCCG
GATCACATGAAACGGCATGACTTTTTCAAGAGTGCCATGCCCGAAGGTTATGTACAGGAACGC
ACTATATATTTCAAAGATGACGGGACCTACAAGACGCGTGCTGAAGTCAAGTTTGAAGGTGAT
ACCCTTGTTAATCGTATCGAGTTAAAGGGTATTGATTTTAAAGAAGATGGAAACATTCTTGGAC
ACAAACTCGAGTACAACTTT
(10) GFP8-5, starting at GFP AA 157
(SEQ ID NO: 60)
ATGAATAATGGAATCAAAGCTAACTTCACAATTCGCCACAACGTTGAAGATGGTTCCGTTCAAC
TAGCAGACCATTATCAACAAAATACTCCAATTGGCGATGGCCCTGTCCTTTTACCAGACGACCA
TTACCTGTCGACACAAACTATCCTTTCGAAAGATCTCAACGAAAAGCGTGACCACATGGTCCTT
CTTGAGTATGTAACTGCTGCTGGGATTACAGATGCTAGCGGTGGAGGGTCTGGTGGCGGATCAA
GGAAAGGAGAAGAACTTTTCACTGGAGTCGTCCCAATTCTTATTGAATTAGATGGTGATGTTAA
TGGGCACAAATTTTTTGTCCGTGGAGAGGGTGAAGGTGATGCTACAATCGGAAAACTCAGCCTT
AAATTTATTTGCACTACTGGAAAACTACCTGTTCCATGGCCAACACTTGTCACTACTCTGACCTA
TGGTGTTCAATGCTTTTCCCGTTATCCGGATCACATGAAACGGCATGACTTTTTCAAGAGTGCCA
TGCCCGAAGGTTATGTACAGGAACGCACTATATATTTCAAAGATGACGGGACCTACAAGACGC
GTGCTGAAGTCAAGTTTGAAGGTGATACCCTTGTTAATCGTATCGAGTTAAAGGGTATTGATTT
TAAAGAAGATGGAAACATTCTTGGACACAAACTCGAGTACAACTTTAACTCACACAAAGTATA
CATCACGGCAGACAAACAA
(11) GFP9-6, starting at GFP AA 173
(SEQ ID NO: 61)
ATGGGTTCCGTTCAACTAGCAGACCATTATCAACAAAATACTCCAATTGGCGATGGCCCTGTCC
TTTTACCAGACGACCATTACCTGTCGACACAAACTATCCTTTCGAAAGATCTCAACGAAAAGCG
TGACCACATGGTCCTTCTTGAGTATGTAACTGCTGCTGGGATTACAGATGCTAGCGGTGGAGGG
TCTGGTGGCGGATCAAGGAAAGGAGAAGAACTTTTCACTGGAGTCGTCCCAATTCTTATTGAAT
TAGATGGTGATGTTAATGGGCACAAATTTTTTGTCCGTGGAGAGGGTGAAGGTGATGCTACAAT
CGGAAAACTCAGCCTTAAATTTATTTGCACTACTGGAAAACTACCTGTTCCATGGCCAACACTT
GTCACTACTCTGACCTATGGTGTTCAATGCTTTTCCCGTTATCCGGATCACATGAAACGGCATGA
CTTTTTCAAGAGTGCCATGCCCGAAGGTTATGTACAGGAACGCACTATATATTTCAAAGATGAC
GGGACCTACAAGACGCGTGCTGAAGTCAAGTTTGAAGGTGATACCCTTGTTAATCGTATCGAGT
TAAAGGGTATTGATTTTAAAGAAGATGGAAACATTCTTGGACACAAACTCGAGTACAACTTTAA
CTCACACAAAGTATACATCACGGCAGACAAACAAAATAATGGAATCAAAGCTAACTTCACAAT
TCGCCACAACGTTGAAGAT
(12) GFP10-7a, starting at GFP AA 189
(SEQ ID NO: 62)
ATGGATGGCCCTGTCCTTTTACCAGACGACCATTACCTGTCGACACAAACTATCCTTTCGAAAG
ATCTCAACGAAAAGCGTGACCACATGGTCCTTCTTGAGTATGTAACTGCTGCTGGGATTACAGA
TGCTAGCGGTGGAGGGTCTGGTGGCGGATCAAGGAAAGGAGAAGAACTTTTCACTGGAGTCGT
CCCAATTCTTATTGAATTAGATGGTGATGTTAATGGGCACAAATTTTTTGTCCGTGGAGAGGGT
GAAGGTGATGCTACAATCGGAAAACTCAGCCTTAAATTTATTTGCACTACTGGAAAACTACCTG
TTCCATGGCCAACACTTGTCACTACTCTGACCTATGGTGTTCAATGCTTTTCCCGTTATCCGGAT
CACATGAAACGGCATGACTTTTTCAAGAGTGCCATGCCCGAAGGTTATGTACAGGAACGCACTA
TATATTTCAAAGATGACGGGACCTACAAGACGCGTGCTGAAGTCAAGTTTGAAGGTGATACCCT
TGTTAATCGTATCGAGTTAAAGGGTATTGATTTTAAAGAAGATGGAAACATTCTTGGACACAAA
CTCGAGTACAACTTTAACTCACACAAAGTATACATCACGGCAGACAAACAAAATAATGGAATC
AAAGCTAACTTCACAATTCGCCACAACGTTGAAGATGGTTCCGTTCAACTAGCAGACCATTATC
AACAAAATACTCCAATTGGC
(13) GFP10-76, starting at GFP AA 195
(SEQ ID NO: 63)
ATGCCAGACGACCATTACCTGTCGACACAAACTATCCTTTCGAAAGATCTCAACGAAAAGCGTG
ACCACATGGTCCTTCTTGAGTATGTAACTGCTGCTGGGATTACAGATGCTAGCGGTGGAGGGTC
TGGTGGCGGATCAAGGAAAGGAGAAGAACTTTTCACTGGAGTCGTCCCAATTCTTATTGAATTA
GATGGTGATGTTAATGGGCACAAATTTTTTGTCCGTGGAGAGGGTGAAGGTGATGCTACAATCG
GAAAACTCAGCCTTAAATTTATTTGCACTACTGGAAAACTACCTGTTCCATGGCCAACACTTGT
CACTACTCTGACCTATGGTGTTCAATGCTTTTCCCGTTATCCGGATCACATGAAACGGCATGACT
TTTTCAAGAGTGCCATGCCCGAAGGTTATGTACAGGAACGCACTATATATTTCAAAGATGACGG
GACCTACAAGACGCGTGCTGAAGTCAAGTTTGAAGGTGATACCCTTGTTAATCGTATCGAGTTA
AAGGGTATTGATTTTAAAGAAGATGGAAACATTCTTGGACACAAACTCGAGTACAACTTTAACT
CACACAAAGTATACATCACGGCAGACAAACAAAATAATGGAATCAAAGCTAACTTCACAATTC
GCCACAACGTTGAAGATGGTTCCGTTCAACTAGCAGACCATTATCAACAAAATACTCCAATTGG
CGATGGCCCTGTCCTTTTA
(14) GFP11-8, starting at GFP AA 214
(SEQ ID NO: 64)
ATGCGTGACCACATGGTCCTTCTTGAGTATGTAACTGCTGCTGGGATTACAGATGCTAGCGGTG
GAGGGTCTGGTGGCGGATCAAGGAAAGGAGAAGAACTTTTCACTGGAGTCGTCCCAATTCTTAT
TGAATTAGATGGTGATGTTAATGGGCACAAATTTTTTGTCCGTGGAGAGGGTGAAGGTGATGCT
ACAATCGGAAAACTCAGCCTTAAATTTATTTGCACTACTGGAAAACTACCTGTTCCATGGCCAA
CACTTGTCACTACTCTGACCTATGGTGTTCAATGCTTTTCCCGTTATCCGGATCACATGAAACGG
CATGACTTTTTCAAGAGTGCCATGCCCGAAGGTTATGTACAGGAACGCACTATATATTTCAAAG
ATGACGGGACCTACAAGACGCGTGCTGAAGTCAAGTTTGAAGGTGATACCCTTGTTAATCGTAT
CGAGTTAAAGGGTATTGATTTTAAAGAAGATGGAAACATTCTTGGACACAAACTCGAGTACAA
CTTTAACTCACACAAAGTATACATCACGGCAGACAAACAAAATAATGGAATCAAAGCTAACTT
CACAATTCGCCACAACGTTGAAGATGGTTCCGTTCAACTAGCAGACCATTATCAACAAAATACT
CCAATTGGCGATGGCCCTGTCCTTTTACCAGACGACCATTACCTGTCGACACAAACTATCCTTTC
GAAAGATCTCAACGAAAAG
(1) GFP2-10, starting at GFP AA 23
(SEQ ID NO: 65)
MGHKFFVRGEGEGDATIGKLSLKFICTTGKLPVPWPTLVTTLTYGVQCFSRYPDHMKRHDFFKSAM
PEGYVQERTIYFKDDGTYKTRAEVKFEGDTLVNRIELKGIDFKEDGNILGHKLEYNFNSHKVYITAD
KQNNGIKANFTIRHNVEDGSVQLADHYQQNTPIGDGPVLLPDDHYLSTQTILSKDLNEKRDHMVLL
EYVTAAGITDASGGGSGGGSRKGEELFTGVVPILIELDGDVN
(2) GFP3-11, starting at GFP AA 39
(SEQ ID NO: 66)
MGKLSLKFICTTGKLPVPWPTLVTTLTYGVQCFSRYPDHMKRHDFFKSAMPEGYVQERTIYFKDDG
TYKTRAEVKFEGDTLVNRIELKGIDFKEDGNILGHKLEYNFNSHKVYITADKQNNGIKANFTIRHNV
EDGSVQLADHYQQNTPIGDGPVLLPDDHYLSTQTILSKDLNEKRDHMVLLEYVTAAGITDASGGGS
GGGSRKGEELFTGVVPILIELDGDVNGHKFFVRGEGEGDATI
(3) GFP4-1a, starting at GFP AA 51
(SEQ ID NO: 67)
MKLPVPWPTLVTTLTYGVQCFSRYPDHMKRHDFFKSAMPEGYVQERTIYFKDDGTYKTRAEVKFE
GDTLVNRIELKGIDFKEDGNILGHKLEYNFNSHKVYITADKQNNGIKANFTIRHNVEDGSVQLADHY
QQNTPIGDGPVLLPDDHYLSTQTILSKDLNEKRDHMVLLEYVTAAGITDASGGGSGGGSRKGEELFT
GVVPILIELDGDVNGHKFFVRGEGEGDATIGKLSLKFICTTG
(4) GFP4-1b, starting at GFP AA 91
(SEQ ID NO: 68)
MYVQERTIYFKDDGTYKTRAEVKFEGDTLVNRIELKGIDFKEDGNILGHKLEYNFNSHKVYITADK
QNNGIKANFTIRHNVEDGSVQLADHYQQNTPIGDGPVLLPDDHYLSTQTILSKDLNEKRDHMVLLE
YVTAAGITDASGGGSGGGSRKGEELFTGVVPILIELDGDVNGHKFFVRGEGEGDATIGKLSLKFICTT
GKLPVPWPTLVTTLTYGVQCFSRYPDHMKRHDFFKSAMPEG
(5) GFP5-2, starting at GFP AA 102
(SEQ ID NO: 69)
MDGTYKTRAEVKFEGDTLVNRIELKGIDFKEDGNILGHKLEYNFNSHKVYITADKQNNGIKANFTIR
HNVEDGSVQLADHYQQNTPIGDGPVLLPDDHYLSTQTILSKDLNEKRDHMVLLEYVTAAGITDASG
GGSGGGSRKGEELFTGVVPILIELDGDVNGHKFFVRGEGEGDATIGKLSLKFICTTGKLPVPWPTLVT
TLTYGVQCFSRYPDHMKRHDFFKSAMPEGYVQERTIYFKD
(6) GFP6-3, starting at GFP AA 117
(SEQ ID NO: 70)
MTLVNRIELKGIDFKEDGNILGHKLEYNFNSHKVYITADKQNNGIKANFTIRHNVEDGSVQLADHY
QQNTPIGDGPVLLPDDHYLSTQTILSKDLNEKRDHMVLLEYVTAAGITDASGGGSGGGSRKGEELFT
GVVPILIELDGDVNGHKFFVRGEGEGDATIGKLSLKFICTTGKLPVPWPTLVTTLTYGVQCFSRYPDH
MKRHDFFKSAMPEGYVQERTIYFKDDGTYKTRAEVKFEGD
(7) GFP7-4a, starting at GFP AA 129
(SEQ ID NO: 71)
MFKEDGNILGHKLEYNFNSHKVYITADKQNNGIKANFTIRHNVEDGSVQLADHYQQNTPIGDGPVL
LPDDHYLSTQTILSKDLNEKRDHMVLLEYVTAAGITDASGGGSGGGSRKGEELFTGVVPILIELDGD
VNGHKFFVRGEGEGDATIGKLSLKFICTTGKLPVPWPTLVTTLTYGVQCFSRYPDHMKRHDFFKSA
MPEGYVQERTIYFKDDGTYKTRAEVKFEGDTLVNRIELKGID
(8) GFP7-4b, starting at GFP AA 140
(SEQ ID NO: 72)
MLEYNFNSHKVYITADKQNNGIKANFTIRHNVEDGSVQLADHYQQNTPIGDGPVLLPDDHYLSTQT
ILSKDLNEKRDHMVLLEYVTAAGITDASGGGSGGGSRKGEELFTGVVPILIELDGDVNGHKFFVRGE
GEGDATIGKLSLKFICTTGKLPVPWPTLVTTLTYGVQCFSRYPDHMKRHDFFKSAMPEGYVQERTIY
FKDDGTYKTRAEVKFEGDTLVNRIELKGIDFKEDGNILGHK
(9) GFP7-4c, starting at GFP AA 145
(SEQ ID NO: 73)
MNSHKVYITADKQNNGIKANFTIRHNVEDGSVQLADHYQQNTPIGDGPVLLPDDHYLSTQTILSKD
LNEKRDHMVLLEYVTAAGITDASGGGSGGGSRKGEELFTGVVPILIELDGDVNGHKFFVRGEGEGD
ATIGKLSLKFICTTGKLPVPWPTLVTTLTYGVQCFSRYPDHMKRHDFFKSAMPEGYVQERTIYFKDD
GTYKTRAEVKFEGDTLVNRIELKGIDFKEDGNILGHKLEYNF
(10) GFP8-5, starting at GFP AA 157
(SEQ ID NO: 74)
MNNGIKANFTIRHNVEDGSVQLADHYQQNTPIGDGPVLLPDDHYLSTQTILSKDLNEKRDHMVLLE
YVTAAGITDASGGGSGGGSRKGEELFTGVVPILIELDGDVNGHKFFVRGEGEGDATIGKLSLKFICTT
GKLPVPWPTLVTTLTYGVQCFSRYPDHMKRHDFFKSAMPEGYVQERTIYFKDDGTYKTRAEVKFE
GDTLVNRIELKGIDFKEDGNILGHKLEYNFNSHKVYITADKQ
(11) GFP9-6, starting at GFP AA 173 (SEQ ID NO: 75)
MGSVQLADHYQQNTPIGDGPVLLPDDHYLSTQTILSKDLNEKRDHMVLLEYVTAAGITDASGGGSG
GGSRKGEELFTGVVPILIELDGDVNGHKFFVRGEGEGDATIGKLSLKFICTTGKLPVPWPTLVTTLTY
GVQCFSRYPDHMKRHDFFKSAMPEGYVQERTIYFKDDGTYKTRAEVKFEGDTLVNRIELKGIDFKE
DGNILGHKLEYNFNSHKVYITADKQNNGIKANFTIRHNVED
(12) GFP10-7a, starting at GFP AA 189
(SEQ ID NO: 76)
MDGPVLLPDDHYLSTQTILSKDLNEKRDHMVLLEYVTAAGITDASGGGSGGGSRKGEELFTGVVPI
LIELDGDVNGHKFFVRGEGEGDATIGKLSLKFICTTGKLPVPWPTLVTTLTYGVQCFSRYPDHMKRH
DFFKSAMPEGYVQERTIYFKDDGTYKTRAEVKFEGDTLVNRIELKGIDFKEDGNILGHKLEYNFNSH
KVYITADKQNNGIKANFTIRHNVEDGSVQLADHYQQNTPIG
(13) GFP10-7b, starting at GFP AA 195 (10-11-linker-1-9)
(SEQ ID NO: 77)
MPDDHYLSTQTILSKDLNEKRDHMVLLEYVTAAGITDASGGGSGGGSRKGEELFTGVVPILIELDGD
VNGHKFFVRGEGEGDATIGKLSLKFICTTGKLPVPWPTLVTTLTYGVQCFSRYPDHMKRHDFFKSA
MPEGYVQERTIYFKDDGTYKTRAEVKFEGDTLVNRIELKGIDFKEDGNILGHKLEYNFNSHKVYITA
DKQNNGIKANFTIRHNVEDGSVQLADHYQQNTPIGDGPVLL
(14) GFP11-8, starting at GFP AA 214 (11-linker-1-10, analogous to 11-linker-1-9-10)
(SEQ ID NO: 78)
MRDHMVLLEYVTAAGITDASGGGSGGGSRKGEELFTGVVPILIELDGDVNGHKFFVRGEGEGDATI
GKLSLKFICTTGKLPVPWPTLVTTLTYGVQCFSRYPDHMKRHDFFKSAMPEGYVQERTIYFKDDGT
YKTRAEVKFEGDTLVNRIELKGIDFKEDGNILGHKLEYNFNSHKVYITADKQNNGIKANFTIRHNVE
DGSVQLADHYQQNTPIGDGPVLLPDDHYLSTQTILSKDLNEK

### Example 4

### Protease Sensor

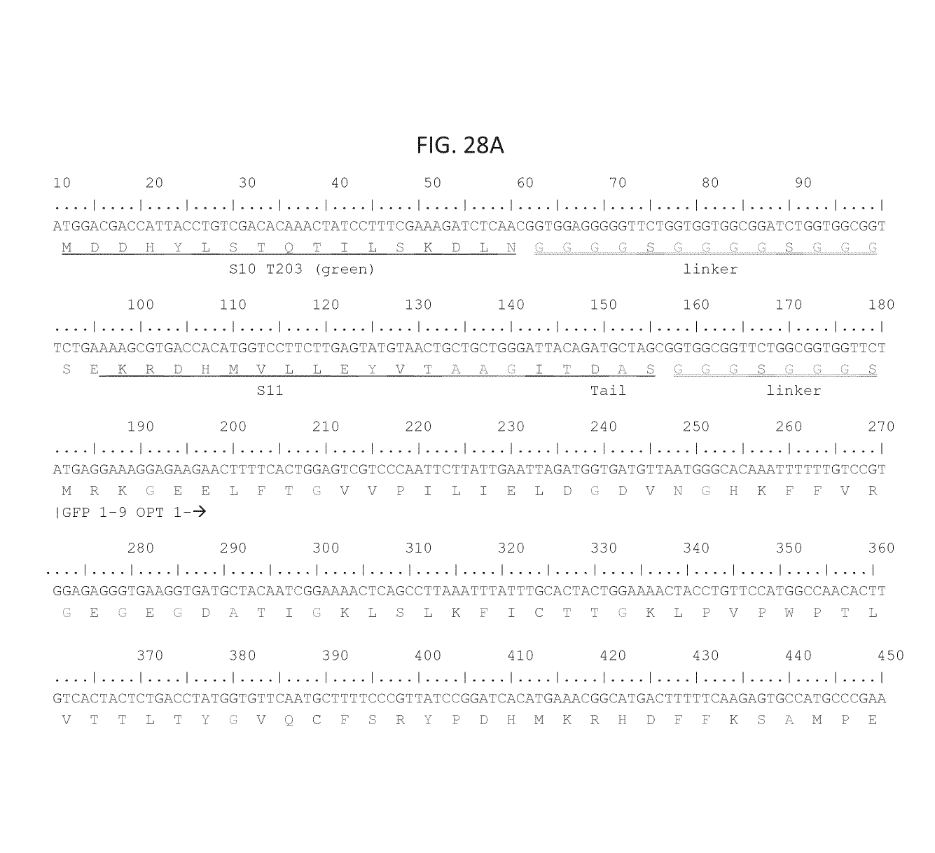

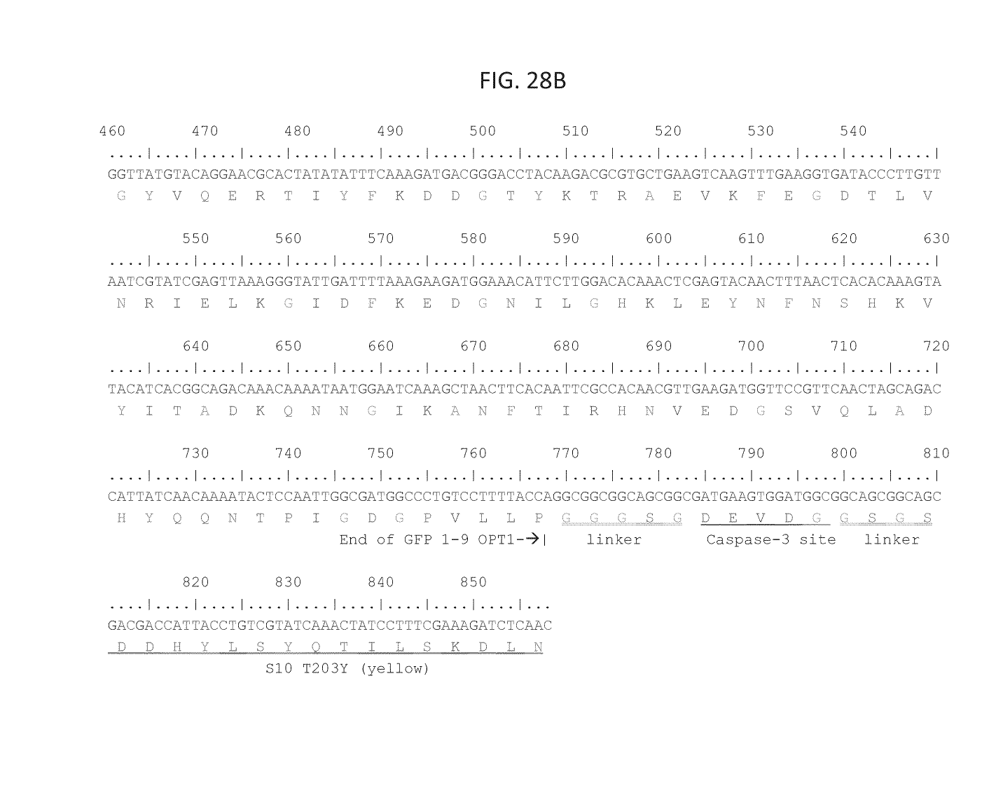

This example illustrates a sensor for caspase-3 activity that is based on the SFP detector/tag embodiments disclosed herein. The sensor is based on a circular permutant of GFP and includes, from N- to C-terminus, β-strand 10, linker, β-strand 11, linker, β-strands 1-9, linker, caspase-3 protease site, linker, and a second copy of β-strand 10 including a T203Y substitution (see FIGS. 28A and 28B). Expression of this construct leads to a predominantly yellow fluorescing protein. Cleavage of the C-terminal yellow strand occurs by caspase-3. The construct remains intact until illuminated by violet light, whereupon the C-terminal strand dissociates, and is replaced by the N-terminal green version (T203) of strand 10, resulting in conversion of yellow fluorescence to green fluorescence, thereby indicating caspase-3 activity. The protease sensor provided in this example includes improved performance as the 1-9 strands have been engineered compared to prior GFP sequences to remain soluble, and the 10 and 11 strands were both engineered compared to prior GFP sequences to minimize perturbation of the folding of fused proteins (which can be the rest of the GFP scaffold itself).

10T203-linker-11-1-9-protease site-10 Y203 nucleotide sequence
(SEQ ID NO: 79)
ATGGACGACCATTACCTGTCGACACAAACTATCCTTTCGAAAGATCTCAACGGTGGAGGGGGTT
CTGGTGGTGGCGGATCTGGTGGCGGTTCTGAAAAGCGTGACCACATGGTCCTTCTTGAGTATGT
AACTGCTGCTGGGATTACAGATGCTAGCGGTGGCGGTTCTGGCGGTGGTTCTATGAGGAAAGG
AGAAGAACTTTTCACTGGAGTCGTCCCAATTCTTATTGAATTAGATGGTGATGTTAATGGGCAC
AAATTTTTTGTCCGTGGAGAGGGTGAAGGTGATGCTACAATCGGAAAACTCAGCCTTAAATTTA
TTTGCACTACTGGAAAACTACCTGTTCCATGGCCAACACTTGTCACTACTCTGACCTATGGTGTT
CAATGCTTTTCCCGTTATCCGGATCACATGAAACGGCATGACTTTTTCAAGAGTGCCATGCCCG
AAGGTTATGTACAGGAACGCACTATATATTTCAAAGATGACGGGACCTACAAGACGCGTGCTG
AAGTCAAGTTTGAAGGTGATACCCTTGTTAATCGTATCGAGTTAAAGGGTATTGATTTTAAAGA
AGATGGAAACATTCTTGGACACAAACTCGAGTACAACTTTAACTCACACAAAGTATACATCACG
GCAGACAAACAAAATAATGGAATCAAAGCTAACTTCACAATTCGCCACAACGTTGAAGATGGT
TCCGTTCAACTAGCAGACCATTATCAACAAAATACTCCAATTGGCGATGGCCCTGTCCTTTTACC
AGGCGGCGGCAGCGGCGATGAAGTGGATGGCGGCAGCGGCAGCGACGACCATTACCTGTCGTA
TCAAACTATCCTTTCGAAAGATCTCAAC
10T203-linker-11-1-9-protease site-10 Y203 amino acid sequence
(SEQ ID NO: 80)
MDDHYLSTQTILSKDLNGGGGSGGGGSGGGSEKRDHMVLLEYVTAAGITDASGGGSGGGSMRKG
EELFTGVVPILIELDGDVNGHKFFVRGEGEGDATIGKLSLKFICTTGKLPVPWPTLVTTLTYGVQCFS
RYPDHMKRHDFFKSAMPEGYVQERTIYFKDDGTYKTRAEVKFEGDTLVNRIELKGIDFKEDGNILG
HKLEYNFNSHKVYITADKQNNGIKANFTIRHNVEDGSVQLADHYQQNTPIGDGPVLLPGGGSGDEV
DGGSGSDDHYLSYQTILSKDLN

It will be apparent that the precise details of the methods or compositions described may be varied or modified without departing from the spirit of the described embodiments. We claim all such modifications and variations that fall within the scope and spirit of the claims below.

It is claimed:

1. A polypeptide, comprising:
at least nine contiguous β-strands of a recombinant green fluorescent protein comprising the consensus amino acid sequence set forth as SEQ ID NO: 1 or a circular permutant thereof.

2. The polypeptide of claim 1, wherein the polypeptide is a Split-Fluorescent Protein (SFP) detector comprising nine contiguous β-strands of the recombinant green fluorescent protein or the circular permutant thereof, wherein the SFP detector can complement with the remaining two β-strands of the SFP to form a fluorescent protein.

3. The polypeptide of claim 2, wherein the SFP detector comprises or consists of the amino acid sequence set forth as SEQ ID NO: 1 (GFP1-9 consensus).

4. The polypeptide of claim 2, wherein the SFP detector comprises or consists of the amino acid sequence set forth as SEQ ID NO: 3 (GFP1-9 OPT1), SEQ ID NO: 2 (GFP1-9 OPT WT), SEQ ID NO: 4 (GFP1-9 OPT2), SEQ ID NO: 65 (GFP2-10 OPT1), SEQ ID NO: 66 (GFP3-11 OPT1), SEQ ID NO: 67 (GFP4-1a OPT1), SEQ ID NO: 68 (GFP4-1b OPT1), SEQ ID NO: 69 (GFP5-2 OPT1), SEQ ID NO: 70 (GFP6-3 OPT1), SEQ ID NO: 71 (GFP7-4a OPT1), SEQ ID NO: 72 (GFP7-4b OPT1), SEQ ID NO: 73 (GFP7-4c OPT1), SEQ ID NO: 74 (GFP8-5 OPT1), SEQ ID NO: 75 (GFP9-6 OPT1), SEQ ID NO: 76 (GFP10-7a OPT1), SEQ ID NO: 77 (GFP10-7b OPT1), or SEQ ID NO: 78 (GFP11-8 OPT1).

5. The polypeptide of claim 3, wherein the SFP detector complements with a Split-Green Fluorescent Protein 10 (GFP10) tag and a Split-Green Fluorescent Protein 11 (GFP11) tag to form the fluorescent protein.

6. The method of claim 5, wherein
the GFP10 tag comprises or consists of the amino acid sequence set forth as SEQ ID NO: 13 (GFP10 M2); and
the GFP11 tag comprises or consists of the amino acid sequence set forth as SEQ ID NO: 14 (GFP11 M4).

7. The polypeptide of claim 1, wherein the polypeptide comprises position 203 of the green fluorescent protein, and wherein the SFP detector further comprises a substitution of a tyrosine residue for a threonine residue at GFP position 203 (T203Y).

8. A nucleic acid molecule encoding the polypeptide of claim 1.

9. The nucleic acid molecule of claim 8, operably linked to a promoter.

10. An expression vector comprising the nucleic acid molecule of claim 9.

11. A host cell comprising the nucleic acid molecule of claim 10.

12. A kit comprising the polypeptide of claim 1 or a nucleic acid molecule encoding the polypeptide, and instructions for using the kit.

13. A method of detecting a protein-protein interaction between a first test polypeptide and a second test polypeptide, comprising:
providing
a SFP detector comprising the polypeptide of claim 2;
a first test polypeptide fused to a first SFP tag comprising the first of the two remaining β-strands; and
a second test polypeptide fused to a second SFP tag comprising the second of the two remaining β-strands; and
wherein the SFP detector, the first SFP tag, and the second SFP tag complement to form a fluorescent protein complex if the first test polypeptide binds to the second test polypeptide; and
detecting fluorescence of the fluorescent protein complex, thereby detecting the protein-protein interaction.

14. The method of claim 13, wherein the SFP detector comprises the amino acid sequence set forth as SEQ ID NO: 1, the first SFP tag is a GFP10 tag, and the second SFP tag is a GFP11 tag.

15. The method of claim 13, wherein providing the GFP1-9 detector, the first test protein fused to the GFP10 tag, and the second test protein fused to the GFP11 tag comprises expressing the SFP detector, the first test protein fused to the first SFP tag, and the second test protein fused to the second SFP tag from one or more expression vectors in a host cell.

16. The method of claim 14, wherein
the GFP10 tag comprises the amino acid sequence set forth as SEQ ID NO: 13 (GFP10 M2) and
the GFP11 tag comprises the amino acid sequence set forth as SEQ ID NO: 14 (GFP11 M4).

17. A polypeptide comprising a fluorescent protein comprising or consisting of the amino acid sequence set forth as SEQ ID NO: 8 (sfCherry).

18. A nucleic acid molecule encoding the polypeptide of claim 17.

19. A polypeptide comprising a protease sensor for caspase-3 activity comprising or consisting of the amino acid sequence set forth as SEQ ID NO: 80.

20. A nucleic acid molecule encoding the polypeptide of claim 19.
